(19) United States
(12) Patent Application Publication
(10) Pub. No.: US 2019/0194612 A1
(43) Pub. Date: Jun. 27, 2019

(54) TRANSPLANTATION EFFECT OF DOPAMINE NEURON ENGRAFTMENT BY CO-TRANSPLANTATION OF MIDBRAIN ASTROCYTES AND VM-NPCS

(71) Applicant: Industry-University Cooperation Foundation Hanyang University, Seoul (KR)

(72) Inventors: Sang-Hun LEE, Seoul (KR); Jaejin SONG, Seoul (KR); Mi-Yoon CHANG, Gyeonggi-do (KR)

(73) Assignee: Industry-University Cooperation Foundation Hanyang University, Seoul (KR)

(21) Appl. No.: 16/180,498

(22) Filed: Nov. 5, 2018

(30) Foreign Application Priority Data
Nov. 6, 2017 (KR) 10-2017-0146708
Nov. 2, 2018 (KR) 10-2018-0133544

(51) Int. Cl. C12N 5/079; C12N 5/0793; A61P 25/28; A61K 35/30

(52) U.S. Cl. CPC C12N 5/0622; C12N 5/0619; C12N 2502/086; A61K 35/30; C12N 2502/081; A61P 25/28

(57) ABSTRACT

The present invention relates to: a cell therapeutic agent including ventral midbrain-derived astrocytes and dopamine neural progenitor cells; a method of treating a neurodegenerative disorder including administering a pharmaceutical composition comprising ventral midbrain-derived astrocytes and dopamine neural progenitor cells into a subject; and a method for differentiation into dopamine neurons, which includes preparing a mixture of ventral midbrain-derived astrocytes and dopamine neural progenitor cells and co-culturing or co-grafting the mixture. The co-grafting of ventral midbrain-derived astrocytes and dopamine neural progenitor cells (neural stem cells) improves the survival and differentiation of dopamine neurons and thus dramatically improves therapeutic outcomes in a neurodegenerative disorder including Parkinson's disease.

### CROSS-REFERENCE TO RELATED APPLICATION

This application claims priority to and the benefit of Korean Patent Application No. 2017-0146708, filed Nov. 6, 2017, the disclosure of which is incorporated herein by reference in its entirety.

### BACKGROUND

### 1. Field of the Invention

The present invention relates to a cell therapeutic agent including ventral midbrain-derived astrocytes and dopamine neural progenitor cells, cells or a cell culture obtained by co-culturing ventral midbrain-derived astrocytes and dopamine neural progenitor cells, a pharmaceutical composition which includes the same to prevent or treat a neurodegenerative disorder, a method of preventing or treating a neurodegenerative disorder using the composition, and a method of co-culturing or co-grafting ventral midbrain-derived astrocytes and dopamine neural progenitor cells and differentiating the cells into dopamine neurons.

### 2. Discussion of Related Art

Given the clinical experience of fetal mesencephalic transplantation in Parkinson's disease (PD), a neurodegenerative disorder characterized by dopamine (DA) neuron loss in the midbrain substantial nigra (SN), stem cell transplantation is regarded as a potential future therapy to treat intractable brain disorders. Neural progenitor cells (NPCs) can be derived from brain tissues or by in vitro differentiation of embryonic stem cells (ESCs) or induced pluripotent stem cells (iPSCs). Therapeutic potentials for cultured NPCs have been shown in both in vitro and in vivo disease models. However, the general consensus is that therapeutic outcomes attained by current NPC transplantation techniques do not reach a satisfactory level to be directly applied to treating patients (Goldman, S. A. 2016. Stem and Progenitor Cell-Based Therapy of the Central Nervous System: Hopes, Hype, and Wishful Thinking. Cell Stem Cell 18:174-188; Steinbeck, J. A., and Studer, L. 2015. Moving stem cells to the clinic: potential and limitations for brain repair. Neuron 86:187-206).

What is ignored also is that the host brain becomes hostile to grafted cells after transplantation due to immunogenic and inflammatory reactions induced by mechanical injury occurring during cell transplantation, which hampers appropriate differentiation, maturation, survival, and function of the grafted cells. Thus, in vitro success in donor NPC cultures efficiently yielding authentic midbrain-type DA neurons with improved survival and functions does not guarantee successful therapeutic outcomes after transplantation, without correcting hostile brain environments.

In order to solve the problems of the prior art, of note, it noted that astrocytes, which outnumber neurons in the CNS (central nervous system), exert physiologic functions to support neuronal cell survival, neuronal function, and brain homeostasis in the adult brain as well as neuronal differentiation and synaptic maturation during development.

The idea of modifying pathologic brain environments by utilizing the neurotrophic properties of astrocytes has been appearing on the therapeutic horizon for CNS disorders. However, multiple properties of astrocytes are compromised in various diseases, and astrocytes can be activated into the type of cells establishing harmful and hostile brain environments in diseased contexts.

A previous study demonstrated that nuclear receptor-related factor 1 (Nurr1; also known as NR4A2), originally known as a transcription factor specific for developing and adult midbrain-type DA (mDA) neurons, could also be expressed in astrocytes/microglia in response to toxic insults, and that Nurr1-expressing glia protect neighboring mDA neurons by reducing synthesis and release of pro-inflammatory cytokines from glial cells[Saijo, K., Winner, B., Carson, C. T., Collier, J. G., Boyer, L., Rosenfeld, M. G., Gage, F. H., and Glass, C. K. 2009. A Nurr1/CoREST pathway in microglia and astrocytes protects dopaminergic neurons from inflammation-induced death. Cell 137:47-59]. Furthermore, we have further shown that forkhead box protein A2 (Foxa2; also known as HNF3β) is a potent cofactor which synergizes the Nurr1-mediated anti-inflammatory roles in glia[Oh, S. M., Chang, M. Y., Song, J. J., Rhee, Y. H., Joe, E. H., Lee, H. S., Yi, S. H., and Lee, S. H. 2015. Combined Nurr1 and Foxa2 roles in the therapy of Parkinson's disease. EMBO Mol Med 7:510-525].

However, there is no report on research of co-grafting of astrocytes and neural progenitor cells.

Therefore, the present inventors have attempted to exploit the neurotrophic actions of astrocytes and/or Nurr1+Foxa2 functions in this cell type to improve the therapeutic outcomes of NPC transplantation using an animal model of neurodegenerative disorder (ex, PD). Our in vitro assays using a co-culture system and conditioned media treatment revealed that astrocytes, especially those cultured from the ventral midbrain (VM) where mDA neurons are developed and reside (hereafter referred as ‘dopaminergic’), greatly support a series of NPC behaviors associated with their therapeutic capacity upon transplantation, such as mDA neuron differentiation, synaptic maturation, midbrain-specific marker expression, presynaptic DA neuron function, and resistance against toxic stimuli. Nurr1+Foxa2 engineering in astrocytes further improved astrocytic function to protect mDA neurons against toxins mainly by reducing inflammation. the present inventors further identified potential neurotrophic cytokines, extracellular matrix proteins, anti-inflammatory and anti-oxidant factors that may mediate the actions of astrocytes. Based on these findings, we demonstrated the functional benefits of astrocyte co-transplantation in NPC-based cell therapy for PD. Therefore, the present invention has been completed based on these facts.

### SUMMARY OF THE INVENTION

Therefore, an object of the present invention is to provide a cell therapeutic agent, which includes ventral midbrain-derived astrocytes and dopamine neural progenitor cells.

In addition, another object of the present invention is to provide a method for differentiation into dopamine neurons, which includes co-culturing or co-grafting ventral midbrain-derived astrocytes and dopamine neural progenitor cells by mixing.

In addition, still another object of the present invention is to provide a method of treating a neurodegenerative disorder, which includes administering a pharmaceutical composition including ventral midbrain-derived astrocytes and dopamine neural progenitor cells to a subject.

In addition, yet another object of the present invention is to provide a cell culture obtained by co-culturing ventral midbrain-derived astrocytes and dopamine neural progenitor cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and advantages of the present invention will become more apparent to those of ordinary skill in the art by describing in detail exemplary embodiments thereof with reference to the attached drawings, in which:

FIG. 1A-FIG. 1M shows that DA neuron differentiation and maturation promoted by co-culturing with astrocytes;

FIG. 1A: Schematic for the co-culture experiments,

FIG. 1B: Representative images of TH+ DA neurons differentiated from VM-NPCs in the presence of Ctx-NPCs (control), Ctx-astrocytes or VM-astrocytes. Insets, Enlarged images of the boxed areas. Scale bar, 100 μm;

FIG. 1C: DA neuronal yields at differentiation day 6 (D6).

FIG. 1D-FIG: 1H Morphometric measurement of DA neuron maturity assessed by neurite outgrowth lengths (FIG. 1D), soma size (FIG. 1E), and by the Sholl test (FIG. 1F, FIG. 1G). In the Sholl analysis, the total number of neurite crossings was counted in each circle with the radius increasing in steps of 15 um (FIG. 1F). The critical value (FIG. 1G) is the radius at which there was a maximum number of neurite crossings. *Significantly different from the control* and Ctx-astrocytes # at p<0.05, n=3-5 wells (FIG. 1C), 100 (FIG. 1D, FIG. 1E), 25-30 (FIG. 1F, FIG. 1G) TH+ cells in each group, One-way ANOVA.

FIG. 1H: Representative TH+ neuronal morphologies reconstructed in 3D by Neurolucida;

FIG. 1I-L: Synaptic densities on TH+ fibers.

FIG. 1I: Representative confocal images of synapsin+/TH+ fibers. Scale bar, 25 Puncta positive for the synaptic vesicle-specific markers SV2 (FIG. 1J), Synapsin (FIG. 1K), and Bassoon (FIG. 1L) on TH+ fibers were counted. Significantly different from the control* and Ctx-astrocytes# at p<0.05, n=20 TH+ fibers for each group.

FIG. 1M: Pre-synaptic DA release, n=3 independent cultures. DA levels were measured in the media conditioned in the differentiated cultures for 24 hrs (D13-15) and in cultures evoked by KCl-mediated depolarization for 30 min. ^{*,#}p<0.05, one-way ANOVA.

FIG. 2A-FIG. 2F shows that DA neurons differentiated with astrocytes are more resistant to toxic insult along with increased midbrain-specific factor expressions;

FIG. 2A-FIG. 2C: Expression of midbrain-specific markers in the differentiated DA neurons. Expression levels of the midbrain-specific markers in (FIG. 2C) were determined in individual TH+ DA neurons by measuring mean fluorescence intensities (MFI) using LAS image analysis (Leica). ^{*,#}p<0.01, one-way ANOVA, n=9 microscopic fields (FIG. 2B) and n=32-36 TH+ cells (FIG. 2C). Scale bar, 50 μm.

FIG. 2D-FIG. 2F: Resistance of DA neurons against a toxic stimulus. TH+ DA neurons differentiated in the presence of Ctx- or VM-astrocytes (Ctx-NPCs as the control) at D12 were exposed to H₂O₂ (1000 μM) for 10 h and viable TH+ cell counts (FIG. 2E) and fiber lengths (FIG. 2F) were measured on the following day. Shown in FIG. 2D is representative TH+ cell images after H₂O₂ treatment. Insets, enlarged images of the boxed areas. Scale bar, 100 μm. Data in E represent % TH+ cells survived after the toxin exposure of those without toxin exposure (H₂O₂=0 μM). *^{,#}p<0.01, one-way ANOVA, n=3 independent experiments (2-3 culture wells/experiment)(FIG. 2E) and n=80-90 TH+ cells (FIG. 2F).

FIG. 3A-FIG. 3N shows that paracrine manner of the astrocyte-mediated neurotrophic actions;

FIG. 3A: Schematic of the conditioned medium (CM) treatment experiments. Media conditioned in Ctx- or VM-astrocytes (or Ctx-NPC as control) was collected and added to cultured VM-NPCs during the differentiation period.

FIG. 3B: Representative images for TH+ DA neurons at D12. Insets, Enlarged images of the boxed areas. Scale bar, 100 μm.

FIG. 3C: TH+DA neuronal yields at D6. *Significantly different from the control at p<0.05, n=3 cultures for each group.

FIG. 3D-FIG. 3E Morphometric measurement of neurite outgrowth assessed by a time-lapse imaging system. VM-NPCs at D3 were treated with the CMs. Neurite lengths (FIG. 3D) and branch points of the neurites (FIG. 3E) in 27 randomly selected microscopic fields from 3 independent cultures were automatically analyzed for 42 hrs using IncuCyte's NeuroTrack software.

FIG. 3F-FIG. 3J: Expression of mature neuronal (NeuN) and mDA neuronal (Foxa2, Nurr1) markers in the differentiated DA neurons. Graphs FIG. 3I and FIG. 3J depict % TH+ cells expressing the markers and the expression levels (MFI) in individual TH+ DA neurons, respectively. Significantly different from the control* and Ctx-astrocytes # at p<0.01, one-way ANOVA, n=9 microscopic fields (FIGS. 3I) and n=32−36 TH+ cells (FIG. 3J).

FIG. 3K-FIG. 3M: Resistance of DA neurons against a toxic stimulus. TH+ DA neurons differentiated in the presence of Ctx- or VM-astrocyte-CM (Ctx-NPC-CM as the control) at D12 were exposed to H₂O₂ (1000 μM) for 8 h and viable TH+ cells were counted on the following day (FIG. 3L). Shown in FIG. 3K are representative TH+ cell images after H₂O₂ treatment. Insets, high-powered images of the boxed areas. Scale bar, 100 μm. Fiber lengths of surviving TH+ cells were also estimated (M). ^{*,#}p<0.01, one-way ANOVA, n=3 independent experiments (2-3 wells/experiment)(FIG. 3L) and n=80-90 TH+ cells (FIG. 3M).

FIG. 3N: Expression of neurotrophic genes in the cultured Ctx-astrocytes, VM-astrocytes, and Ctx-NPCs, estimated by real-time PCR (qPCR) analyses. ^{*,#}p<0.05, one-way ANOVA, n=3.

FIG. 4A-FIG. 4F shows that potential molecules mediating the dopaminotrophic functions of astrocytes;

FIG. 4A-FIG. 4E: mRNA-seq analyses for the differentially expressed genes (DEGs) among VM-astrocytes, Ctx-astrocytes, and control Ctx-NPCs. Genes up/down regulated by over 2-fold were selected for analysis.

FIG. 4A: Venn-diagram summarizing the overlap between DEGs from VM-astrocytes vs. Ctx-NPCs (left circle) and Ctx-astrocytes vs. Ctx-NPCs (right circle). The numbers of genes are indicated in the Venn-diagram. To calculation of overlap with DEGs, statistical analysis was performed using the Chi-square test based on a two-by-two table using R version 3.3.2 with MASS package.

FIG. 4B: GO and KEGG analyses for the 4445 overlapping genes (‘common astrocytic genes’). The dark gray bar indicates the number of genes under the designated GO term/KEGG pathway. The light gray bar indicates the p-value, and the negative log of the p-value (bottom) is plotted on the X-axis.

FIG. 4C-FIG. 4D: Volcano plot (FIG. 4C) and GO/KEGG analyses (FIG. 4D) for the DEGs between VM-astrocytes and Ctx-astrocytes. E, Heat-map showing fold changes of the selected genes in VM- and Ctx-astrocytes compared to control Ctx-NPCs (Log 2 folds (VM-astrocyte/Ctx-NPC) and (Ctx-astrocyte/Ctx-NPC)).

FIG. 4F: Expression of important pro- and anti-inflammatory and secretory anti-oxidant genes was further confirmed by qPCR analyses. Significantly different from the control* and Ctx-astrocytes # at p<0.05, n=3 PCR reactions, ANOVA.

FIG. 5A-FIG. 5L shows that forced expression of Nurr1+Foxa2 in VM-astrocytes potentiates the astrocyte-mediated dopaminotrophic actions;

FIG. 5A: Schematized procedure of the co-culture experiments. VM-astrocytes were transduced with Nurr1+Foxa2-expressing lentiviruses (or control mock viruses), harvested, and mixed with VM-NPCs. After differentiation was induced in the VM-NPCs mixed with the astrocytes, TH+ DA neuronal yields (FIG. 5B), morphologic (FIG. 5C) and synaptic maturation (FIG. 5D), expression of midbrain-specific markers (FIG. 5E), presynaptic DA release (FIG. 5F), and resistance to a toxic stimulus (FIG. 5G, FIG. 5H) were assessed in the TH+ DA neurons in the differentiated cultures.

FIG. 5I-FIG. 5L: CM treatment experiments. CM was prepared from Nurr1+Foxa2-transduced VM-astrocytes (or mock-transduced VM-astrocytes as control), and added to VM-NPCs during the differentiation period. The differentiated cultures at D12 were exposed to H₂O₂ (1000 μM, 10 hrs) and the number (FIG. 5K) and neurite length (FIG. 5L) of surviving TH+ cells were estimated as described in the FIG. 3 legend. ^{*,#}p<0.05, 2-tailed Student's t-test, n=3 culture wells (FIG. 5F, FIG. 5H, FIG. 5K) and 80-90 TH+ cells (FIG. 5L). Scale bars, 100 μm].

FIG. 6A-FIG. 6F shows that RNA-seq analysis for the DEGs between Nurr1+Foxa2− and control VM-astrocytes.

FIG. 6A: GO and KEGG analyses for the genes upregulated (log 2>1) or downregulated (log 2<−1) in Nurr1+Foxa2-VM-astrocytes (NF-Ast) compared to control VM-astrocytes (Cont-Ast) (FPKM>1).

FIG. 6B: Gene ontologies only for the genes downregulated in NF-Ast vs. control-Ast (log 2<−1, FPKM>1).

FIG. 6C: GSEA for ‘immune response’ and ‘inflammatory response’ gene sets enriched in Nurr1+Foxa2-VM-astrocytes vs VM-astrocytes.

FIG. 6D: Heat-map of the expression levels (FPKMs) of all the genes annotated to the ontologies related to immune (121 genes)/inflammatory response (103 genes) in control-Ast vs. NF-Ast in 'FIG. 6B.

Expressions of selected immune/inflammatory, inhibitory ECMs and anti-oxidant genes are further shown in 'FIG. 6E. FIG. 6F, mRNA levels of important pro-/anti-inflammatory and neurotrophic factors were further confirmed by real-time qPCR analyses. *p<0.05, 2-tailed Student's t-test, n=3.

FIG. 7A-FIG. 7B shows that improvement of host brain environments by grafting cultured astrocytes; Rats were transplanted with VM-astrocytes, Ctx-astrocytes, or Ctx-NPCs (control);

FIG. 7A: qPCR data for gene expressions associated with neurotrophic or hostile brain environments among the grafted groups. The PCR analyses were carried out in the graft-host interfaces dissected as described in the ‘Materials & Methods’ at 1 month of post-transplantation. *,#p<0.05, n=3.

FIG. 7B: Immunohistochemical analyses for the glial cells immunoreactive for pro-inflammatory/cytotoxic (iNOS, CD11b, CD16) and anti-inflammatory/neuroprotective (Arginase, CD206) factors. The immunoreactive cells along the graft-host interfaces were counted in 6 cryosectioned slices from 3 animals/each group at 7-10 days of post-transplantation. Data are expressed as % immunoreactive cells out of DAPI+ cells, of GFAP+ astrocytic, and of Iba1+ microglial populations. *,#p<0.05, n=6, ANOVA. Scale bar, 100 μm.

FIG. 8A-FIG. 8B shows that effects of Nurr1+Foxa2 priming in donor VM-astrocytes to improve host brain environments after transplantation. [Neurotrophic or inflammatory factor expressions in the brains grafted were determined by qPCR (FIG. 8A) and immunohistochemical (FIG. 8B) analyses as described in FIG. 7. *,#p<0.05, n=3 PCRs (FIG. 8A) and 6 slices (FIG. 8B). 2-tailed t-test. Scale bars, 100 μm]

FIG. 9A-FIG. 9L shows that co-grafting of astrocytes improves the therapeutic effects of VM-NPC transplantation in a PD rat model.[NPCs derived from rat embryonic VM at E12 were expanded in vitro, harvested and mixed with Ctx-NPCs (control), Ctx-astrocytes, VM-astrocytes, or N+F-VM-astrocytes before cell injection. The mixed cells were intrastriatally transplanted into 6-OHDA-lesioned PD model rats. Behavioral (FIG. 9A-FIG. 9C) and histological (FIG. 9D-FIG. 9L) analyses were carried out during or at 6 months post-transplantation. A, Amphetamine-induced rotation scores during 6 months post-transplantation. Data are given as percent changes in rotation scores for each animal compared with the pre-transplantation value. Means+SEMs of the rotation scores are depicted. n=6 for each group. Significant decreases from the co-grafted with the control NPCs*, Ctx-Ast #, VM-Ast† at each post-transplantation time point at p<0.05, ANOVA. Behaviors of the transplanted animals were further assessed by step-adjustment (FIG. 9B) and cylinder (FIG. 9C) tests at 6 months post-transplantation. Statistical significances (p<0.01) among groups are expressed using schematics in the graphs. One-way ANOVA followed by Bonferroni post hoc analysis. FIG. 9D-FIG. 9L, Histologic analyses 6 months after transplantation. FIG. 9D, Overview of the TH+ cell grafts. FIG. 9E, Graft volume. FIG. 9F, Total number of TH+ cells. FIG. 9G TH+ cell density in the graft. *,#p<0.05, ANOVA. FIG. 9H-J, Morphologic maturation of DA neurons in the grafts estimated by TH+ fiber length. Shown are immunohistochemical (FIG. 9H) and Neurolucida reconstruction of representative TH+ neuronal images (FIG. 9I). FIG. 9K-FIG. 9L, Synaptic maturation of TH+ DA neurons estimated by synapsin+ puncta density. *,#p<0.05, ANOVA. Scale bars, 100 μm (FIG. 9D), 50 μm (FIG. 9H) 25 μm (FIG. 9K)].

FIG. 10 shows that schematic summary for the trophic actions of astrocytes co-grafted in PD cell therapeutic approach [Astrocytes (VM, N+F-VM-astrocytes) co-grafted with VM-NPCs promotes a series of transplanted NPC survival, mDA neuron differentiation, neuronal maturation, and synaptic integration by correcting hostile host brain environments. The astrocytic actions are attained via secretion of various neurotrophic ECM proteins, anti-oxidants, neurotrophic cytokines, GLAST/GLT1-mediated clearance of glutamate toxicity, and etc. Ultimately, PD behaviors are improved along with enriched engraftment of mature and functional mDA neurons expressing midbrain-specific markers.].

FIG. 11A-FIG. 11J shows that immature and brain region-specific identities of the astrocytes cultured from mouse cortices and VM;

FIG. 11A and FIG. 11B: Expression of astrocytic markers estimated by immunocytochemical (FIG. 11A) and real-time qPCR (FIG. 11B) analyses. Undifferentiated NPCs derived from mouse embryonic cortices (Ctx-NPC) were used as the control. Data present mean±SEM of four (FIG. 11A) and three (FIG. 11B) experiments. Scale bar, 50 um. C, Gene expression levels of immature and mature astrocytic markers in the cultured astrocytes, estimated by FPKM in the RNA-seq data. The FPKM value is shown above each bar in the graph.

FIG. 11D-FIG. 11F: Patch-clamp electrophysiological analysis on cultured astrocytes derived from cortex and VM at DIV 14 and 35. Shown in (FIG. 11D) are representative traces for the voltage ramp-induced whole-cell passive conductance currents recorded from each cell. FIG. 11E: Summary bar graph of average conductance. Number of cells analyzed is indicated in each bar. P-values were obtained with one-way ANOVA (Kruskal-Wallis test). *P<0.05. FIG. 11F: Percentage of the number of cells showing low conductance (<10 nS) from each group.

FIG. 11G-FIG. 11H: Regenerative capacity of cultured astrocytes associated with astrocyte maturity were assessed after scratch injury (FIG. 11G) and in an inhibitory proteoglycan environment (FIG. 11H). Scratch injury (FIG. 11G) and aggrecan/laminin gradient spots (FIG. 11H) were generated on cultured Ctx- and VM-astrocytes at DIV12-14 and 33-35 as described in the Materials and Methods. % areas filled by astrocytic cells between the two edges of the scratch (FIG. 11G) and numbers of cells that grew inward the spots (FIG. 11H) were measured during 2 days. Shown are the representative phase-contrast and GFAP-immunofluorescent images of VM astrocytes at DIV12-14 and 33-35. *p<0.01, n=6, Student t-test. Scale bars, 100 μm.

FIG. 11I and FIG. 11J: Region-specific gene expression in the cultured Ctx- and VM-astrocytes was assessed by RNA-seq (FIG. 11I) and qPCR (FIG. 11J) analyses. Significantly different from the Ctx-NPC control* and Ctx-astrocytes # at p<0.05, n=3 PCR reactions, ANOVA.

FIG. 12A-FIG. 12B shows that glutamatergic excitatory and GABAergic inhibitory synaptic formation;

FIG. 12A: Immunocytochemistry of dendrites in TH+ fibers of cultured mDA neurons colabeled with antibodies against glutamatergic presynaptic marker vesicular glutamate transporter 2 (vGlut2) and the excitatory post-synaptic marker post-synaptic density protein 95 (PSD95) or the GABAergic presynaptic marker vesicular GABA transporter (vGAT) and the inhibitory post-synaptic marker Gephyrin to visualize pre- and postsynaptic elements of excitatory or inhibitory synapses, respectively. Arrowheads point to vGlut2+/PSD95+ or vGAT+/Gephyrin+clusters.

FIG. 12B: Quantification of the synaptic puncta. Data represent mean±SEM. ***p<0.01 by ANOVA followed by Fisher's LSD post hoc test, n=8−16 for excitatory and 9-15 for inhibitory synapses. Scale bars, 2 μm.

FIG. 13 shows that gene set enrichment analyses (GSEA) for high-ranked GO and KEGG pathways in FIG. 4B and FIG. 4D.

FIG. 14A-FIG. 14B shows that neuroprotective activities of cultured astrocytes by eliminating oxidants and glutamate-mediated toxicity;

FIG. 14A: Anti-oxidant capacity assessed by intracellular glutathione levels.

FIG. 14B: Glutamate uptake activity. Undifferentiated and differentiated Ctx-NPCs (after 6 days of diff) were used as the negative controls for glutamate uptake assays. *,#p<0.05, n=4 (A), 3 (B), ANOVA.

FIG. 15A-FIG. 15C shows that effects of forced Nurr1+Foxa2 expression in VM-astrocyte-mediated ROS scavenging and glutamate clearance activities;

FIG. 15A; Intracellular glutathione levels in control- vs. NF-astrocytes.

FIG. 15B: ROS levels in mDA neuron cultures treated with NF-astrocyte CM or control-astrocyte CM. mDA neuron-enriched cultures were derived by differentiation of VM-NPCs and treated with CM prepared from N+F− or mock control-transduced VM-astrocytes. After cells were exposed to H₂O₂ (500 uM, 3 hrs), ROS levels, detected by DCF staining, were quantified by DCF+ cell counting. Scale bar, 100 um.

FIG. 15C: Glutamate uptake activity. *p<0.05, n=3, Student's t-test (2-tailed).

FIG. 16A-FIG. 16C shows that co-expression of mature neuronal (HuC/D) and midbrain-specific mDA neuronal (Foxa2 and Nurr1) markers in TH+ DA cells at 6 months post-transplantation.

FIG. 16D˜FIG. 16E shows that representative TH+/GFAP+ and TH+/Iba1+ cell images in the grafts. Scale bars, 25 um.

FIG. 17A-FIG. 17D shows that DA neurotrophic actions elicited by cultured VM-astrocytes in the absence or presence of minor microglia population. Pure (no Iba+ and O4+ cells) and enriched VM astrocyte cultures with minor microglia contamination (Iba+ cells: <0.5%) were prepared as described in the Materials and Methods.

FIG. 17A: Representative images for GFAP+/Iba1+/DAPI+ (left) and Iba+/DAPI+ (right) cells.

FIG. 17B-FIG. 17D: E12 VM-NPCs during differentiation were treated with the CM prepared from the astrocyte cultures. mDA neuron differentiation, morphologic maturation, and resistance to toxic insult were assessed by TH+ mDA neuron yields at D6 (FIG. 17B), TH+ neurite lengths at D12 (FIG. 17C), and viable TH+ cells survived after H₂O₂ treatment (1000 uM, 8 hrs) at D12 (FIG. 17D), respectively. *,#p<0.05, n=6, Scale bar, 50 μm.

FIG. 18A-FIG. 18C shows that effects of viral transduction on astrocyte-mediated neurotrophic actions[VM-astrocytes (non-transduced), those transduced with mock control and with Nurr1+Foxa2-expressing lentiviruses were subjected to qPCR analyses to assess expressions of neurotrophic factors and pro-inflammatory cytokines (FIG. 18A). CMs were prepared from the non-transduced and transduced VM-astrocytes. In the presence of the CM supplementation, cell viability against H₂O₂ toxin (FIG. 18B) and pre-synaptic DA release (FIG. 18C) were assessed in the cultures differentiated from VM-NPCs at differentiation day 12. Significantly different from the non-transduced* and mock-transduced # controls at p<0.05, ANOVA, n=4. Scale bar, 50 μm.].

FIG. 19A-FIG. 19B shows that pan-trophic actions of cultured astrocytes [VM-NPCs were differentiated in the absence or presence of CM prepared in cultured Ctx- or VM-astrocytes. Yields of total TuJ1+ neurons and GFAP+ astrocytes (FIG. 19A) and glutamatergic (vGlut2+/TuJ1+) and GABAergic (GABA+) neuronal subtypes (FIG. 19B) were assessed. *Significantly different from the untreated control at p<0.05, ANOVA, n=4. Scale bar, 50 μm.].

FIG. 20 shows comparison of mRNA levels of phagocytosis- and autophagy-related genes through RNA-Seq using astrocytes isolated from the cortex (cortex-type astrocytes; Ctx-astrocytes), astrocytes isolated from the ventral midbrain (ventral midbrain-type astrocytes; VM-astrocytes), and neural stem cells isolated from the cortex (Ctx-NPC) as the control.

FIG. 21 shows that astrocyte co-exsistance decreased the α-synuclein accumulation (pathology). α-synuclein-overexpressing ventral midbrain dopamine neurons obtained by differentiation of ventral midbrain neural stem cells (VM-NPC) transduced with α-synuclein-overexpressing lentiviruses are co-cultured with each of Ctx-astrocytes, VM-astrocytes and the control (Ctx-NPC, which are neural stem cells isolated from the cortex) and treated with PFF (pre-formed fibrils), which is an α-synuclein aggregate, during differentiation to induce α-synuclein aggregation, and then, 20 days after the differentiation, the aggregation is detected with thioflavin S and α-synuclein by α-synuclein/thioflavin S immunocytochemistry (thioflavin S: staining for detection of pathologic protein aggregates).

FIG. 22 shows the accumulation of α-synuclein oligomers analyzed by western blotting. Ventral midbrain neural stem cells (VM-NPC) transduced with α-synuclein-overexpressing lentiviruses were co-cultured with each of Ctx-astrocytes, VM-astrocytes and the control (Ctx-NPC, which are neural stem cells isolated from the cortex) and treated with PFF (pre-formed fibrils). At 20 days differentiation, cells were extracted and fractioned to triton X-100 (1%)-soluble and insoluble fraction. α-synuclein oligomers are indicated with a bracket, while monomer bands are marked with an arrowhead.

FIG. 23 shows that α-synuclein is overexpressed in SH-SY5Y cells, co-cultured with non-overexpressed SH-SY5Y cells and treated with a conditioned medium harvested from Ctx-astrocytes, VM-astrocytes or the control (Ctx-NPC, which are neural stem cells isolated from the cortex), and then transmission of α-synuclein is confirmed by determining expression levels of α-syn-GFP.

FIG. 24 is a schematic diagram of a PD model in which overexpression is induced in the striatum of a mouse using PFF, which is an α-synuclein aggregate, and Lenti α-syn viruses.

FIG. 25 shows a result of observing TH/p a-syn (the pathologic form of α-syn) after co-grafting in the striatum of an α-synuclein-overexpressing mouse. [The left image shows the co-grafting of ventral midbrain neural progenitor cells (VM-NPC) and ventral midbrain-derived astrocytes (VM-Ast), and the right image shows the single grafting of ventral midbrain neural progenitor cells (VM-NPC). The leftmost and rightmost images are the enlarged images of the respective grafts.]

FIG. 26 shows a result of observing TH/ThS (pathologic protein aggregate straining) after co-grafting in the striatum of an α-synuclein-overexpressing mouse. [The left image shows the co-grafting of ventral midbrain neural progenitor cells (VM-NPC) and ventral midbrain-derived astrocytes (VM-Ast), and the right image shows the single grafting of ventral midbrain neural progenitor cells (VM-NPC). The leftmost and rightmost images are the enlarged images of the respective grafts.]

FIG. 27 shows a result of observing GFAP (astrocytes straining)/ThS (pathologic protein aggregate staining) after co-grafting in the striatum of an α-synuclein-overexpressing mouse. [The left image shows the co-grafting of ventral midbrain neural progenitor cells (VM-NPC) and ventral midbrain-derived astrocytes (VM-Ast), and the right image shows the single grafting of ventral midbrain neural progenitor cells (VM-NPC). The leftmost and rightmost images are the enlarged images of the respective grafts.]

FIG. 28 shows a result of observing human α-synuclein (human α-synuclein staining)/ThS (pathologic protein aggregate staining) after human ventral midbrain neural progenitor cells and astrocytes are co-grafted in the striatum of an α-synuclein-overexpressing mouse. [The left image shows the co-grafting of ventral midbrain neural progenitor cells (VM-NPC) and ventral midbrain-derived astrocytes (VM-Ast), and the right image shows the single grafting of ventral midbrain neural progenitor cells (VM-NPC). The leftmost and rightmost images are the enlarged images of the respective grafts.]

FIG. 29 shows that α-synuclein is overexpressed in SH-SY5Y cells, co-cultured with non-overexpressed SH-SY5Y cells and treated with a conditioned medium obtained from Ctx-astrocytes, VM-astrocytes or the control (neural stem cells isolated from the cortex, Ctx-NPC), and then α-synuclein transmission is confirmed by determining expression levels of α-syn.

FIG. 30 shows that α-synuclein is overexpressed in SH-SY5Y cells, co-cultured with each of α-syn-GFP and α-syn-mCherry and treated with a conditioned medium obtained from Ctx-astrocytes, VM-astrocytes or the control (neural stem cells isolated from the cortex, Ctx-NPC), and then α-synuclein aggregation is confirmed by determining expression levels of α-syn.

FIG. 31 shows the accumulation of α-synuclein oligomers analyzed by western blotting. Ventral midbrain neural stem cells (VM-NPC) transduced with α-synuclein-overexpressing lentiviruses were co-cultured with Ctx-astrocytes, VM-astrocytes or the control (neural stem cells isolated from the cortex, Ctx-NPC) while treatment of a conditioned medium, and treated with PFF (pre-formed fibrils). At 20 days differentiation, cells were extracted and fractioned to triton X-100 (1%)-soluble and insoluble fraction. α-synuclein oligomers are indicated with a bracket, while monomer bands are marked with an arrowhead.

FIG. 32 shows that secreted factors from astrocytes have effect for decreasing α-synuclein aggregation. α-synuclein-overexpressing ventral midbrain dopamine neurons obtained by differentiation of ventral midbrain neural stem cells (VM-NPC) transduced with α-synuclein-overexpressing lentiviruses are co-cultured with Ctx-astrocytes, VM-astrocytes or the control (neural stem cells isolated from the cortex, Ctx-NPC) while treatment of a conditioned medium, and are treated with PFF (pre-formed fibrils), which is an α-synuclein aggregate, during differentiation to induce α-synuclein aggregation, and then, 20 days after the differentiation, the aggregation is detected by α-synuclein immunocytochemistry and quantified.

FIG. 33 shows that secreted factors from astrocytes have effect for decreasing α-synuclein aggregation (pathology). α-synuclein-overexpressing ventral midbrain dopamine neurons obtained by differentiation of ventral midbrain neural stem cells (VM-NPC) transduced with α-synuclein-overexpressing lentiviruses are co-cultured with Ctx-astrocytes, VM-astrocytes or the control (neural stem cells isolated from the cortex, Ctx-NPC) while treatment of a conditioned medium, and are treated with PFF (pre-formed fibrils), which is an α-synuclein aggregate, during differentiation to induce α-synuclein aggregation, and then, 20 days after the differentiation, the aggregation is detected with thioflavin S and α-synuclein by α-synuclein/thioflavin S immunocytochemistry (thioflavin S: staining for detection of pathologic protein aggregates).

FIG. 34 shows the result of western blotting to detect α-synuclein and aggregation in cells and supernatants after Ctx-astrocytes, VM-astrocytes or the control (neural stem cells isolated from the cortex, Ctx-NPC) are cultured with and without treatment with PFF (an α-synuclein aggregate) for 24 hours.

FIG. 35 shows a result of observing CD11b (M1-type staining)/Iba1 (microglia staining) after grafting to the striatum of a rat. [The left image shows the grafting of human ventral midbrain neural progenitor cells (VM-NPC), and the right image shows the grafting of human ventral midbrain-derived astrocytes (VM-Ast). The leftmost and rightmost images are the enlarged images of the respective grafts.]

FIG. 36 shows a result of observing Arg1 (M2-type staining)/Iba1 (microglia staining) after grafting to the striatum of a rat. [The left image shows the grafting of human ventral midbrain neural progenitor cells (VM-NPC), and the right image shows the grafting of human ventral midbrain-derived astrocytes (VM-Ast). The leftmost and rightmost images are the enlarged of the respective grafts.]

FIG. 37 shows a result of observing stained TH dopaminergic neurons after grafting to the striatum of a rat. [The left image shows the grafting of human ventral midbrain neural progenitor cells (VM-NPC), and the right image shows the grafting of human ventral midbrain-derived astrocytes (VM-Ast). The leftmost and rightmost images are the enlarged images of the respective grafts.]

### DETAILED DESCRIPTION OF EXEMPLARY EMBODIMENTS

Hereinafter, exemplary embodiments of the present invention will be described in detail. However, the present invention is not limited to the embodiments disclosed below and can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting the exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof and do not preclude the presence or addition of one or more other features, integers, steps, operations, elements, components and/or groups thereof.

With reference to the appended drawings, exemplary embodiments of the present invention will be described in detail below. To aid in understanding of the present invention, like numbers refer to like elements throughout the description of the figures, and the description of the same elements will not be iterated.

The terms used in the present invention are defined as follows.

The present invention provides a cell therapeutic agent including ventral midbrain-derived astrocytes and dopamine neural progenitor cells.

That is, ventral midbrain-derived astrocytes and dopamine neural progenitor cells are co-grafted and are differentiated into dopamine neurons, with an ultimate purpose to treat a neurodegenerative disorder.

The “co-grafting” used herein refers to the in vivo grafting of both of astrocytes and dopamine neural progenitor cells, and it includes separately storing the different types of cells, mixing the cells together immediately before use and administering the mixture to a subject to be treated through injection.

To confirm a therapeutic effectiveness of such “co-grafting” in vitro, in the exemplary embodiment, a co-culture of astrocytes and dopamine neural progenitor cells is used in an experiment.

The “glia” used herein refers to cells accounting for the largest portion of cells present in the brain, and it includes “astrocytes” or “microglia.” The astrocytes are also known as astroglia and are involved in the protection, nourishment, and inflammation of neuronal cells, and the microglia are cells responsible for inflammation in the brain.

The astrocytes may be obtained by differentiation from embryonic or adult stem cells, or by separation from the ventral midbrain, cerebral cortex or lateral ganglionic eminence (striatum anlage) of a mammal. In the present invention, as the “astrocytes,” ventral midbrain (VM)-type astrocytes are used.

The “neural stem cells (NSCs)” or “neural progenitor cells (NPCs)” are separated and cultured from embryonic stem cells, developing or adult brain tissue, and the cultured NPCs may be used in mass-production of dopamine neurons for research and drug screening.

The term “neuron” refers to a cell type of the central nervous system, and the terms “neuron” and “neuronal cell” may be interchangeably used herein.

In addition, it is preferable that VM-astrocytes and NPCs are mixed at a cell number ratio of 1:1.5˜3 to adjust a practical co-grafting amount.

Particularly, in the present invention, it was also confirmed that the overexpression of Nurr1 and Foxa2 in donor astrocytes further promotes the neurotrophic action of grafted astrocytes in a cell-based therapy. That is, the survival and differentiation of dopamine neurons were enhanced by co-grafting of Nurr1+Foxa2-overexpressing VM-astrocytes and dopaminergic NSCs, and thus the therapeutic outcomes were dramatically improved. Here, NSCs or NPCs were transduced with FoxA2 and Nurr1 to overexpress the same.

The “transduction” is a phenomenon in which a genetic trait is transferred from a cell to another cell via a bacteriophage, thereby introducing the genetic trait to the latter. When a bacteriophage infects a certain type of bacterium, phage DNA binds to host DNA, and as the phage is removed from the bacterium due to bacteriolysis, it may take out a part of the host DNA while losing a part of its own DNA instead. When the phage infects another bacterium, the former host gene is newly introduced into the bacterium, and therefore, the bacterium exhibits a new trait. The term “transduction” used in biological research generally refers to the overexpression of a specific exogenous gene in a target cell using a viral vector. In other words, the transduction may be carried out using a viral vector, preferably, a lentivirus or retrovirus vector.

In addition, in an exemplary embodiment of the present invention, an inhibitory effect of α-synuclein aggregation and transmission by astrocytes was confirmed. In addition, in an exemplary embodiment of the present invention, an effect of reducing and improving inflammation by human astrocytes differentiated from human embryonic cells was confirmed.

The present invention also provides a method for differentiation into dopamine neurons, which includes co-culturing or co-grafting ventral midbrain-derived astrocytes and dopamine neural progenitor cells by mixing.

The “dopamine (DA) neuron” refers to a neuron that expresses tyrosine hydroxylase (TH). The terms “dopaminergic neuron,” “dopamine neuron,” and “DA” are used interchangeably herein. The DA neuron is specifically located in the midbrain substantia nigra, and controls postural reflexes, motions and compensation-related behaviors by stimulating striatum, limbic system and neocortex in vivo.

The term “differentiation” refers to the phenomenon in which structures and functions are specialized while cells are proliferated by division and grown, and in other words, the change in form or function of cells or tissue of an organism to accomplish its given task.

The present invention also provides a pharmaceutical composition for preventing or treating a neurodegenerative disorder, which includes VM-derived astrocytes and dopamine NPCs.

The term “prevention” used herein means all actions of inhibiting a neurodegenerative disorder or delaying the onset thereof by administration of the composition of the present invention, and the term “treatment” used herein means all actions involved in alleviating or beneficially changing symptoms of a neurodegenerative disorder by administration of the composition of the present invention.

The “treatment” used herein refers to all actions involved in preventing, alleviating or beneficially changing clinical situations related to a disease. In addition, the treatment may refer to an increased survival compared with an expected survival rate when untreated. The treatment includes a preventive means in addition to a therapeutic means.

In this specification, the term “subject” may refer to a vertebrate to be tested for treatment, observation or experiments, preferably a mammal, for example, a cow, a pig, a horse, a goat, a dog, a cat, a rat, a mouse, a rabbit, a guinea pig, a human, etc.

The composition of the present invention is effective in the prevention or treatment of a neurodegenerative disorder. The neurodegenerative disorder may include various neurological diseases, for example, PD, dementia, Alzheimer's disease, Huntington's disease, amyotrophic lateral sclerosis, memory impairment, myasthenia gravis, progressive supranuclear palsy, multiple system atrophy, essential tremor, cortico-basal ganglionic degeneration, diffuse Lewy body disease and Pick's disease, but the present invention is not limited thereto.

The composition of the present invention may be applied as a cell therapeutic agent, and it may be formulated by further including a pharmaceutically acceptable carrier. The term “pharmaceutically acceptable carrier” used herein refers to a carrier or diluent which does not significantly irritate an organism and does not interfere with the biological activity and properties of an administered ingredient. In the present invention, a pharmaceutically acceptable carrier for the composition of the present invention that can be applied as a cell therapeutic agent may be any one known in the art such as a buffer, a preservative, an analgesic, a solubilizer, an isotonic agent, a stabilizer, a base, an excipient or a lubricant without limitation. The pharmaceutical composition of the present invention may be prepared in various formulations according to a technique commonly used. The composition of the present invention, which is the cell therapeutic agent, may be administered by any route as long as it can induce transmission to a disease lesion. In some cases, a method for loading the composition into a vehicle, which includes a means for directing stem cells to a lesion, may be considered. Therefore, the composition of the present invention may be administered by various routes including local administration (including buccal, sublingual, skin and intraocular administration), non-oral administration (including subcutaneous, intradermal, intramuscular, infusion, intravenous, intraarterial, intraarticular and intrathecal administration), and transdermal administration, and preferably, it is directly administered to a disease lesion. In an aspect, stem cells may be suspended in a suitable diluting agent and administered into a subject, and the diluting agent is used to protect and maintain cells, and to facilitate use in injection into desired tissue. The diluting agent may be a buffer solution such as a physiological saline, a phosphate buffered solution, or HBSS, or a cerebrospinal fluid. In addition, the pharmaceutical composition may be administered by a random device to transmit an active ingredient to target cells. A preferable administration method and type of preparation is an injection. The injection may be prepared using an aqueous solvent such as a physiological saline, a Ringer's solution, a Hank's solution or a sterilized aqueous solution, or a non-aqueous solvent such as a vegetable oil (e.g., olive oil), a higher fatty acid ester (e.g., ethyl oleate), ethanol, benzyl alcohol, propylene glycol, polyethylene glycol or glycerin, and for mucosal permeation, a non-permeable agent known in the art and suitable for a barrier to be permeated may be used, and ascorbic acid, sodium hydrogen sulfite, BHA, tocopherol or EDTA may be used as a stabilizer for preventing spoilage, and a pharmaceutical carrier such as an emulsifier, a buffer for pH adjustment, or a preservative for preventing microbial development such as mercury nitrate, thimerosal, benzalkonium chloride, phenol, cresol or benzyl alcohol may be additionally included.

The term “cell therapeutic agent” refers to a medicine (U.S. FDA regulations) used for the purpose of treatment, diagnosis and prophylaxis using cells and tissues prepared through isolation from a human, culturing and special homogenization, that is, a medicine used for the purpose of treatment, diagnosis and prophylaxis through a series of actions of proliferating and selecting living autologous, allogenic or xenogenic cells in vitro to restore the functions of cells or tissues, or changing biological characteristics of cells by another method. Cell therapeutic agents are mainly classified into somatic cell therapeutic agents and stem cell therapeutic agents, depending on a differentiation level of the cells.

In the present invention, the term “administration” means that a composition according to the present invention is introduced to a subject or a patient using any suitable method. In this case, the composition according to the present invention may be administered through various routes of oral or parenteral administration as long as the composition can reach target tissue. The composition may be intraperitoneally, intravenously, intramuscularly, subcutaneously, intradermally, orally, topically, intranasally, intrapulmonarily, and rectally administered, but the present invention is not limited thereto.

The term “administration” used herein means the introduction of the composition of the present invention to a patient by any suitable method, and an administration route of the composition of the present invention may vary as long as the composition can reach desired tissue, and it may be any one of various routes including oral and non-oral routes. The composition of the present invention may be administered intraperitoneally, intravenously, intramuscularly, subcutaneously, intradermally, orally, locally, intranasally, intrapulmonarily or intrarectally, but the present invention is not limited thereto.

In this specification, the term “effective amount” refers to a desired amount required to delay or completely interrupt the onset or progression of a certain disease to be treated. The cells may be administered at a daily dose of 1.0×10⁴ to 1.0×10¹⁰ cells/kg body weight, preferably at 1.0×10⁵ to 1.0×10⁹ cells/kg body weight once or several times a day. However, it should be understood that an actual dose of an active ingredient has to be determined by various related factors including a disease to be treated, the severity of a disease, an administration route, a patient's body weight, age and sex, and therefore, the dose does not limit the scope of the present invention in any aspect.

Therefore, the present invention provides a cell therapeutic agent including ventral midbrain-derived astrocytes and dopamine neural progenitor cells, a pharmaceutical composition for preventing or treating a neurodegenerative disorder, which includes cells or a cell culture obtained by co-culturing ventral midbrain-derived astrocytes and dopamine neural progenitor cells, and a method of treating a neurodegenerative disorder, which includes administering the composition to a subject.

The present invention also includes a cell culture obtained by co-culturing ventral midbrain-derived astrocytes and dopamine neural progenitor cells.

In the cell culture, an increase in the neurotrophic factors and survival of DA neurons and ventral midbrain-derived astrocytes, Nurr1 and Foxa2-induced reduction in inflammation and improvement in viability of DA neurons thereby were observed. Therefore, the cell culture may be used as an active ingredient of a cell therapeutic agent or pharmaceutical composition for a neurodegenerative disorder.

Hereinafter, the present invention will be described in detail with reference to examples thereof. However, it should be understood that the following examples are just preferred examples for the purpose of illustration only and is not intended to limit or define the scope of the invention. The following examples described herein are provided in order to make the present invention more comprehensive and complete and provide the scope of the present invention to those skilled in the art to which the present invention belongs and thus will be defined by the appended claims equivalents thereof.

### EXAMPLES

[Materials and Methods]

1. Cell Culture

NPC Culture

NPCs with DA neurogenic potential were cultured from the VMs of mouse embryos (imprinting control region, ICR) at E(embryonic day) 10.5 or Sprague-Dawley rat embryos at E12. The VM-NPCs were expanded in serum-free N2 medium supplemented with the mitogens basic fibroblast growth factor (bFGF; 20 ng/ml; R&D Systems, Minneapolis, Minn., USA) and epithelial growth factor (EGF; 20 ng/ml; R&D Systems, only for mouse cultures) to reach a confluency of >70% (usually for 3-4 days) and passaged. After an additional NPC expansion, cells were harvested for co-culture and other experiments or directly induced to differentiate by withdrawing the mitogens (in CM treatment experiments). NPCs were also cultured from the cortex, a non-dopaminergic brain region (mouse at E12 or rat at E14) and used as control cells in the following experiments.

Astrocyte Culture

Astrocytes were isolated from mouse or rat VMs or cortices (Ctx) on postnatal day 5-7 and cultured in astro-medium [Heinrich, C., Gascon, S., Masserdotti, G., Lepier, A., Sanchez, R., Simon-Ebert, T., Schroeder, T., Gotz, M., and Berninger, B. 2011. Generation of subtype-specific neurons from postnatal astroglia of the mouse cerebral cortex. Nat Protoc 6:214-228.]. In brief, VMs were removed, triturated in Dulbecco's modified Eagle's medium (DMEM; Life Technologies, Camarillo, Calif., USA) containing 10% fetal bovine serum (FBS; HyClone, Logan, Utah), and plated in 75-cm2 T-flasks. When cell confluence reached 80-90%, cells were harvested with 0.1% trypsin and sub-cultured on a culture surface coated with poly-D-lysine (PDL; Sigma, ST, Louis, Mo.). Four-6 days later, microglia were eliminated by shaking at 180 rpm on an orbital shaker. After culturing for 7 days, astrocytes were harvested for co-culture experiments or further cultured for an additional 8 days in N2 to prepare the conditioned medium (CM).

As described above, minor populations of microglia were present even after the microglia elimination procedure. The astrocyte culture containing a minor microglia population was used in this study. To estimate the effects of minor microglia contamination, microglia-free astrocyte culture was established by treating the cultures with 0.06% trypsin in DMEM/F12 for 20-30 min after the shaking procedure, and discarding floating cells.

Co-Culture

VM-NPCs with DA neurogenic potential were harvested and mixed with the Ctx-astrocytes or VM-astrocytes at a 2:1 ratio of VM-NPCs to astrocytes (3×10⁴ vs 2×10⁴ based on the 24-well plate). The mixed cells were plated and differentiation of VM-NPCs was directly induced in serum-free N2 medium. In the control cultures, VM-NPCs were mixed with non-dopaminergic Ctx-NPCs.

Human Ventral Midbrain Neural Progenitor Cells (or Human Ventral Midbrain Neural Stem Cells, Human VM NPC)

For direct differentiation into human ventral midbrain neural progenitor cells, hESCs (human Embryonic Stem cells, e.g., WA-09, H9 (purchased from WiCell)) were treated with LDN193189 (100 nM, Stemgent), SB431542 (10 μM, Tocris), SHH (100 ng/ml, R&D), purmorphamine (2 μM, Stemgent), FGF8 (100 ng/ml, R&D) and CHIR99021 (CHIR; 3 μM, Stemgent) for 17 days in order of ventral mid-brain patterning. The cells were plated at a proper cell density (2×10⁶ cells per 6-cm dish), cultured in a Matrigel (BD)-coated dish for 20 days, and then cultured in a poly-L-ornithine (PLO)/fibronectin (FN)/laminin-coated dish at a proper cell density (3×10⁶ per 6-cm dish).

Culture of Human Ventral Midbrain Astrocytes

To culture human ventral midbrain astrocytes directly differentiated from human ventral midbrain neural progenitor cells as described above, cells were plated at a cell density (3×10⁶ cells per 6-cm dish), cultured in a Matrigel (BD)-coated dish for 20 days and subcultured in a poly-L-ornithine (PLO)/fibronectin (FN)/laminin-coated dish at a proper cell density (3×10⁶ per 6-cm dish) for approximately 120 days. To confirm the culture, a small amount of the cells was plated in a 24-well plate before use, and then differentiation was determined by GFAP immunocytochemistry.

Conditioned Medium (CM) Treatment

As described above, fresh N2 medium (or HBSS(Hank's balanced salt solution)) was added to the astrocyte cultures, and CM of the astrocyte was collected every other day for 8 days. The control CM was similarly prepared in the Ctx-NPC cultures during 6 days of differentiation. The volume of the CMs was adjusted to 0.1-0.15 mg of protein/ml, filtered at 0.45 μm, and kept at −80° C. until use. The CMs were diluted with N2 medium (1:1, v:v) before adding to the cells in culture.

Nurr1+Foxa2 Transduction

Lentiviral vectors expressing Nurr1 or Foxa2 under the control of the CMV promoter were generated by inserting the respective cDNA into the multi-cloning site of pCDH (System Biosciences, Mountain View, Calif.). The empty backbone vector (pCDH) was used as a negative control. The lentiviruses were produced and used for transducing in vitro cultures as described [Yi, S. H., He, X. B., Rhee, Y. H., Park, C. H., Takizawa, T., Nakashima, K., and Lee, S. H. 2014. Foxa2 acts as a co-activator potentiating expression of the Nurr1-induced DA phenotype via epigenetic regulation. Development 141:761-772.]. Titers of the lentiviruses were determined using a QuickTiter™ HIV Lentivirus Quantitation Kit (Cell Biolabs, San Diego, Calif., USA), and 20 ul each of the Nurr1 and Foxa2 viruses with 10⁶ transducing units (TU)/mL were mixed with 2 mL of media and added to 1-1.5×10⁶ cells/6cm-dish for the transduction reaction.

Immunostaining

Cultured cells were fixed with 4% paraformaldehyde (PFA) in phosphate-buffered saline (PBS), and blocked in 0.3% Triton X-100 with 1% bovine serum albumin (BSA) for 40 minutes, then incubated with primary antibodies overnight at 4° C. Primary antibody information is shown in Table 1. The secondary antibodies used for visualization were: Cy3 (1:200, Jackson Immunoresearch Laboratories, West Grove, Pa., USA) or Alexa Fluor 488 (1:200, Life Technologies).

TABLE 1

| | | | |
|---|---|---|---|
| | Working | | |
| Antibodies | dilution | Company | Catalog numbers |

Mouse monoclonal Antibody (Ab)

| | | | |
|---|---|---|---|
| Bassoon | 1:400 | ⁶Enzo life science | ADI-VAM- |
| | | | PS003-D |
| CD16/32 | 1:500 | ²BD Biosciences | 553142 |
| CD44 | 1:500 | ¹¹Novus Biologicals | .NBP1-31488 |
| Gephyrin | 1:500 | ¹⁶Synaptic Systems | 147 011 |
| GFAP | 1:200 | ⁸MP Biomedicals | 691102 |
| GLAST | 1:1000 | ⁹Miltenyl Biotec | 130-095-821 |
| HuC/D | 1:100 | ⁷Chemicon | A21271 |
| iNOS | 1:1000 | ²BD Biosciences | 610432 |
| NeuN | 1:200 | ¹⁰Millipore | MAB377 |
| Nurr1 | 1:1000 | ¹³R&D Systems | PP-N1404-00 |
| Synapsin1 | 1:1000 | ²BD Biosciences | 611392 |
| SV2 | 1:50 | ⁵DHSB | 2315387 |
| Synaptophysin | 1:1000 | ²BD Biosciences | 611880 |
| Tyrosine | 1:200 | ⁷Immunostar | T-1299 |
| Hydroxylase (TH) | | | |

Rabbit Polyclonal Ab

| | | | |
|---|---|---|---|
| CD206 | 1:500 | ¹Abcam | ab64693 |
| GFAP | 1:400 | ⁴DAKO | Z0334 |
| GLT-1 | 1:200 | ¹Abcam | ab106289 |
| lba-1 | 1:200 | ¹⁵Wako | 019-19741 |
| Lmx1a | 1:2000 | ¹⁰Millipore | AB10533 |
| Nurr1 | 1:500 | ¹⁴Santa Cruz | sc-991 |
| TH | 1:1000 | ¹²Pel-freez | P40101 |
| S100β | 1:500 | ¹Abcam | ab868 |
| Sox2 | 1:100 | ³Chemicon | AB5603 |
| vGAT | 1:1000 | ¹⁶Synaptic Systems | 131 003 |
| Vglut2 | 1:1000 | ¹⁶Synaptic Systems | 135 403 |

Goat Polyclonal Ab

| | | | |
|---|---|---|---|
| Arg1 | 1:500 | ¹¹Novus Biologicals | NB100-59740 |
| Foxa2 | 1:500 | ¹⁴Santa Cruz | sc-6554 |
| PSD95 | 1:500 | ¹⁶Synaptic Systems | 124 014 |

Rat Polyclonal Ab

| | | | |
|---|---|---|---|
| CD11B | 1:500 | ¹⁰Millipore | CBL1512 |

Chicken Polyclonal Ab

| | | | |
|---|---|---|---|
| Tyrosine | 1:500 | ¹Abcam | ab76442 |
| Hydroxylase (TH) | | | |

The stained cells were mounted with VECTASHIELD with DAPI mounting solution (Vector Laboratories, Calif., USA) and photographs were obtained by an epifluorescence microscope (Leica, Heidelberg, Germany) and confocal microscope (Leica PCS SP5).

Transplantation and Histological Procedures

Experiments were performed in accordance with National Institutes of Health (NIH) guidelines. Hemi-parkinsonian was induced in adult female Sprague-Dawley rats (220-250 g) by unilateral stereotactic injection of 3 μl of 6-hydroxydopamine (6-OHDA, 8 μg/μl; Sigma) into the right side of the substantia nigra (AP—4.8 mm, ML—1.8 mm, V—8.2 mm) and the median forebrain bundle (AP—4.4 mm, ML—1.2 mm, V—7.8 mm). The incisor bar was set at −3.5 mm, and AP and ML coordinates are given relative to bregma. Rats with 300 turns/hr ipsilateral to the lesion in an amphetamine-induced rotation test were selected. For transplantation, rat E12 VM-NPCs were expanded and mixed with the Ctx-, VM-astrocytes, N+F-VM-astrocytes or E14 Ctx-NPCs (control) at a 2:1 ratio. Three ul of the mixed cells (1.5×10⁵ cells/ul) were injected over a 10 min period into each of two sites in the striatum (coordinates in AP, ML, and V relative to bregma and dura: [1] 0.07, −0.30, −0.55; [2] −0.10, −0.40, −0.50; incisor bar set at 3.5 mm below zero) under anesthesia induced by Zoletil 100 ul/100 g (50 mg/ml) mixed with Rompun 100 ul/100 g (23.32 mg/ml). The needle (22 gauge) was left in place for 5 min after the completion of each injection. Rats received daily injections of cyclosporine A (10 mg/kg, i.p.) starting 1 day before the grafting and continuing for 1 month and were maintained without the immunosuppressant for the rest of the post-transplantation period. Six months after transplantation, animals were anesthetized and perfused transcardially with 4% paraformaldehyde. Brains were removed and immersed in 30% sucrose in PBS overnight, frozen in TissueTek® (Sakura Finetek, Torrance, Calif., USA), and then sliced on a freezing microtome (Leica). Free-floating brain sections (30 μm thick) were subjected to immunohistochemistry as described above and images were obtained with a confocal microscope (Leica). In the experiment to examine host environments of the transplanted brains, animals were sacrificed at 1 month post-transplantation and grafted brains were sliced at a thickness of 1 mm on a rat brain slice matrix (ZIVIC Instruments, Pittsburgh, Pa.), and to observe a change after grafting, tissue was extracted from an engrafted site. 8-12 regions of the graft-host interface (ca 2×2 mm)/graft were dissected and subjected to qPCR analyses. Cells immunoreactive for neurotrophic and pro-inflammatory glial markers were also counted along the graft-host interfaces of cryosectioned brain slices at 7-10 days post-transplantation.

Behavior Tests

Animal behaviors were assessed using amphetamine-induced rotation, step adjustment, and cylinder tests as previously described [Oh, S. M., Chang, M. Y., Song, J. J., Rhee, Y. H., Joe, E. H., Lee, H. S., Yi, S. H., and Lee, S. H. 2015. Combined Nurr1 and Foxa2 roles in the therapy of Parkinson's disease. EMBO Mol Med 7:510-525].

Cell Counting and Statistical Analysis

Immunostained and DAPI-stained cells were counted in 9-20 random areas of each culture coverslip using an eyepiece grid at a magnification of 200× or 400×. For every figure, data are expressed as the mean±SEM and statistical tests are justified as appropriate. Statistical comparisons were made using the Student's t-test (unpaired), 2-tailed or one-way ANOVA followed by Bonferroni post hoc analysis using SPSS (Statistics 21; IBM Inc. Bentonville, Ark., USA). The n, p-values, and statistical analysis methods are indicated in the figure legends. A P value less than 0.05 was considered significant.

Study Approval

All procedures for animal experiments were approved by the Institutional Animal Care and Use Committee (IACUC) at Hanyang College of Medicine, Seoul, Korea (approval number 2016-0113A).

Astrocyte Functional Assays

Electrophysiological Recording in Cultured Astrocytes

Cultured astrocytes were plated onto coverslips and maintained in DMEM supplemented with 10% fetal bovine serum and 10% horse serum for at least 24 h before electrophysiology experiments. For electrophysiological recording, the patch pipettes had an open-tip resistance of 4-7MΩ when filled with a pipette solution containing (in mM): 150 KCl, 1 CaCl2, 1 MgCl2, 5 EGTA and 10 HEPES (pH 7.2 was adjusted with KOH). Standard bath solution contained (in mM): 150 NaCl, 3 KCl, 2 CaCl2, 1 MgCl2, HEPES, 5.5 D-glucose and 20 10 sucrose (pH 7.4was adjusted with NaOH). The mean membrane resistance of hippocampal astrocytes in culture was 7.45±0.76 mega ohms (mean±s.e.m.), and the series resistance was <30 mΩ, which we monitored throughout all experiments. Recording pipettes were fabricated from borosilicate glass capillaries (World Precision Instruments) using a P-97 Flaming/Brown micropipette puller (Sutter Instruments).Whole-cell membrane currents were amplified by the Axopatch 200A. Currents were elicited by 1-s ramps from −150 mV to +50mV (from a holding potential of −70 mV). Data acquisition was controlled by signals between amplifier and computer. Data were sampled at 5 kHz and filtered at 2 kHz. Cell membrane capacitance was measured by using the membrane test protocol built into pClamp10.0. The calculated and measured junction potentials were −2.6mV and −2.5 mV, respectively.

Scratch Injury Assay

Using a sterile pipette tip, scratches were made on the monoconfluent cell layer of astrocytes. The plates were then rinsed with sterile PBS to remove cell debris and replaced with fresh cell culture media. At 0, 1 and 2 days after scratch, the area occupied by astrocytes were measured between the two edges of the scratch using Image J software.

Growing Capacity in Inhibitory Proteoglycan Environment

Gradient spot glass coverslips (12 mm) were prepared as described previously [Tom, V. J., Steinmetz, M. P., Miller, J. H., Doller, C. M., and Silver, J. 2004. Studies on the development and 22 behavior of the dystrophic growth cone, the hallmark of regeneration failure, in an in vitro model of the glial 23 scar and after spinal cord injury. J Neurosci 24:6531-6539.]. Briefly, coverslips coated with poly-L-lysine (PLL; Sigma-Aldrich, St. Louis, Mo.) and nitrocellulose were spotted with 2 ul of a solution of aggrecan (0.7 mg/mL; Sigma-Aldrich) and laminin (5 ug/mL; Biomedical Technologies, Stoughton, Mass.) in Ca++, Mg++-free Hank's Balanced Salt Solution (CMF; Invitrogen, Gaithersburg, Md.). The spots were allowed to dry and were then covered with laminin (5 ug/mL) in CMF and kept at 37° until just before cell plating (for 3 h). The laminin solution was removed before plating astrocytes (2×104 cells/well in 24-well plate). Migratory potentials of astrocytes inward the gradient aggrecan/laminin spots were determined by the areas covered by astrocytes in the spots during 2 days.

Morphometric Assessments

To estimate morphological maturation, total fiber length and soma size (perimeter) of randomly selected TH+ DA neurons were measured using an image analysis system (Leica LAS). TH+ DA neuron images were also reconstructed using Neurolucida 360 (MicroBrightfield, Inc.). Complexity of the fibers in TH+ cells was further assessed using the Sholl test. The number of intersections of the neurite tree with increasing perimeters from the center of the soma was counted every 15 μm up to a distance of 200 μm. The critical value of the radius at which there is a maximum number of neurites was also determined in the Sholl test. Neurite length and branching of TH+ DA neurons were further assessed using time-lapse imaging as described below.

Time-Lapse Imaging of Neuron Axonal Elongation

VM-NPCs from the mouse embryos at E10.5 were seeded at 4.0×104 cells in each well of a 24-well plate and cultured. CMs were treated from 3 days after differentiation and diluted with N2 medium (1:1, v:v) to induce neurite elongation. Phase contrast microscopic images were automatically taken using the IncuCyte ZOOM Live Cell Imaging System (Essen Bioscience, Ann Arbor, Mich., USA) every 2 h for 42 hours. Neurite lengths and branch points of the neurites were automatically analyzed with IncuCyte's NeuroTrack software.

Messenger RNA Expression Analysis

Total RNA was prepared using the Trizol Reagent (Invitrogen, Carlsbad, Calif., USA) through the RNA isolation protocol. cDNA synthesis was carried out using a Superscript kit(Invitrogen). Real-time PCR was performed on a CFX96™ Real-Time System using iQ™ SYBR green supermix (Bio-Rad, Hercules, Calif., USA) and gene expression levels were determined relative to GAPDH levels. The Mouse Oxidative Stress RT² Profiler™ PCR Array (cat. 330231 PAMM-065ZA) was used to profile the expression of 84 genes related to oxidative stress using a RT2 Profiler PCR ArrayR (Qiagen, Gaithersburg, Md., USA). Primer information is shown in Table 2.

TABLE 2

| Gene | Primer Sequence(Sense/Antisense) | |
|---|---|---|
| AHR | GTC CTC AGC AGG AAC GAA AG | CCA GGG AAG TCC AAC TGT GT |
| | (SEQ ID NO: 1) | (SEQ ID NO: 2) |
| AQP4 | CGG TTC ATG GAA ACC TCA CT | CAT GCT GGC TCC GGT ATA AT |
| | (SEQ ID NO: 3) | (SEQ ID NO: 4) |
| ARG1 | TAT CGG AGC GCC TTT CTC TA | ACA GAC CGT GGG TTC TTC AC |
| | (SEQ ID NO: 5) | (SEQ ID NO: 6) |
| BDNF | GTG ACA GTA TTA GCG AGT GGG | GGG TAG TTC GGC ATT GC |
| | (SEQ ID NO: 7) | (SEQ ID NO: 8) |
| CCL17 | AAT GTA GGC CGA GAG TGC TG | CAT GCT TGT CTT TGG GGT CT |
| | (SEQ ID NO: 9) | (SEQ ID NO: 10) |
| CCL22 | TTC TTG CTG TGG CAC TTC AG | CTT CTT CAC CCA GAG CAT CC |
| | (SEQ ID NO: 11) | (SEQ ID NO: 12) |
| COL6A2 | ATG GAC AGA AGG GCA AAC TG | CTT GCC TCC TTT CAC TCC TG |
| | (SEQ ID NO: 13) | (SEQ ID NO: 14) |
| CXCL9 | CTC ATG GGC ATC ATC TTC CT | TCA GCT TCT TCA CCC TTG CT |
| | (SEQ ID NO: 15) | (SEQ ID NO: 16) |
| CXCL10 | TCG TGC TGC TGA GTC TGA GT | GGC TCA CCG CTT TCA ATA AG |
| | (SEQ ID NO: 17) | (SEQ ID NO: 18) |
| CXCL11 | TAT GAT CAT CTG GGC CAC AA | CCA GGC ACC TTT GTC CTT TA |
| | (SEQ ID NO: 19) | (SEQ ID NO: 20) |
| EPO | CCA GCC ACC AGA GAG TCT TC | TGC AGA AAG TAT CCG CTG TG |
| | (SEQ ID NO: 21) | (SEQ ID NO: 22) |
| FGF8 | TGT TGC ACT TGC TGG TTC TC | ACT CGG ACT CTG CTT CCA AA |
| | (SEQ ID NO: 23) | (SEQ ID NO: 24) |
| FIZZ1 | ATC TGC GTC TTC CTT CTC CA | CAG TAG CAG TCA TCC CAG CA |
| | (SEQ ID NO: 25) | (SEQ ID NO: 26) |
| FN1 | GAA AGG CAA CCA GCA GAG TC | CTG GAG TCAAGC CAG ACA CA |
| | (SEQ ID NO: 27) | (SEQ ID NO: 28) |
| Foxa2 | GCT CCC TAC GCC AAT ATC AA | CCG GTA GAA AGG GAA GAG GT |
| | (SEQ ID NO: 29) | (SEQ ID NO: 30) |
| Foxg1 | GAA CGG CAA GTA CGA GAA GC | TCA CGA AGC ACT TGT TGA GG |
| | (SEQ ID NO: 31) | (SEQ ID NO: 32) |
| GDNF | CGC CCG CCG AAG ACC ACT CC | GTC GAA GGC GAC CGG CCT GC |
| | (SEQ ID NO: 33) | (SEQ ID NO: 34) |
| GFAP | GCA GAC CTC ACA GAC GTT GCT | AGG CTG GTT TCT CGG ATC TGG |
| | (SEQ ID NO: 35) | (SEQ ID NO: 36) |
| GLT1 | ATC CTG GGA GCA GTA TGT GG | CTG ACA GCC CTG TGA TGA GA |
| | (SEQ ID NO: 37) | (SEQ ID NO: 38) |
| GPC4 | AGT GCC TTC AGT GCT CGA TT | CTT CCC GTT CCAACA GTC AT |
| | (SEQ ID NO: 39) | (SEQ ID NO: 40) |
| GPC6 | AGC CAGATACCTGCCTGAGA | TCA TTG CCA TTG TAC GCA TT |
| | (SEQ ID NO: 41) | (SEQ ID NO: 42) |
| GPX3 | ACC AAT TTG GCA AAC AGG AG | AGC GGA TGT CAT GGA TCT TC |
| | (SEQ ID NO: 43) | (SEQ ID NO: 44) |
| Hevin | ATT GGC AAC CAG AAG GAC AC | GGT TCT CAC TCT CGC CAG TC |
| | (SEQ ID NO: 45) | (SEQ ID NO: 46) |
| IFN-α | GGT GGT GGT GAG CTA CTG GT | TTG AGC CTT CTG GAT CTG CT |
| | (SEQ ID NO: 47) | (SEQ ID NO: 48) |
| IFN-β | CTG CCCTCTCCATCGACTAC | ACC CAG TGC TGG AGA AAT TG |
| | (SEQ ID NO: 49) | (SEQ ID NO: 50) |
| IL-10 | CCT GCT CTT ACT GGC TGG AG | TGT CCA GCT GGT CCT TCT TT |
| | (SEQ ID NO: 51) | (SEQ ID NO: 52) |
| IL-12b | ACC CTC ACC TGT GAC AGT CC | TTC TTG TGG AGC AGC AGA TG |
| | (SEQ ID NO: 53) | (SEQ ID NO: 54) |
| IL-13 | ATC GAG GAG CTG AGC AAC AT | CGA GGC CTT TTG GTT ACA GA |
| | (SEQ ID NO: 55) | (SEQ ID NO: 56) |
| IL-19 | AGA AGG CAT GAA GGC ACA GT | TCA CGC AGC ACA CAT CTA CA |
| | (SEQ ID NO: 57) | (SEQ ID NO: 58) |
| IL-1R2 | CAT GGG AGA TGC AGG CTA TT | ACA CCT TGC ACG GGA CTA TC |
| | (SEQ ID NO: 59) | (SEQ ID NO: 60) |
| IL-1RN | GAA AAG ACC CTG CAA GAT GC | GAT GCC CAA GAA CAC ATT CC |
| | (SEQ ID NO: 61) | (SEQ ID NO: 62) |
| IL-1β | CTG TGA CTC GTG GGA TGA TG | GGG ATT TTG TCG TTG CTT GT |
| | (SEQ ID NO: 63) | (SEQ ID NO: 64) |
| IL-2RA | CCA GAG AGT GAG GCT TCC TG | ACT CAG GAG GAG GAT GCT GA |
| | (SEQ ID NO: 65) | (SEQ ID NO: 66) |
| IL-4 | TCC TTA CGG CAA CAA GGA AC | GTG AGT TCA GAC CGC TGA CA |
| | (SEQ ID NO: 67) | (SEQ ID NO: 68) |
| IL-6 | ATG GAT GCT ACC AAA CTG GAT | TGA AGG ACT CTG GCT TTG TCT |
| | (SEQ ID NO: 69) | (SEQ ID NO: 70) |
| iNOS | CAC CTT GGA GTT CAC CCA GT | ACC ACT CGT ACT TGG GAT GC |
| | (SEQ ID NO: 71) | (SEQ ID NO: 72) |
| ITGαM | TTA CCG GAC TGT GTG GAC AA | AGT CTC CCA CCA CCA AAG TG |
| | (SEQ ID NO: 73) | (SEQ ID NO: 74) |
| ITGβ4 | GAA GGA ACT GCA GGT GAA GC | GCG ATG CGG ATA TCT CAT TT |
| | (SEQ ID NO: 75) | (SEQ ID NO: 76) |
| Lmx1a | CCC CAA AAT CCG GAA TTA CT | CTCAGGGTCAGCAAAAGGAG |
| | (SEQ ID NO: 77) | (SEQ ID NO: 78) |
| NF1A | ACC CGA GTA CCG AGA GGA TT | ACT GTG GGG ACT TCA CAA GG |
| | (SEQ ID NO: 79) | (SEQ ID NO: 80) |
| NT3 | GGT CAG AAT TCC AGC CGA TGA | GGC ACA CAC ACA GGA AGT GTC |
| | (SEQ ID NO: 81) | (SEQ ID NO: 82) |
| Nurr1 | ACC CTC TTC TGG ACA CAT GG | GCCATCACCACAAGACACAC |
| | (SEQ ID NO: 83) | (SEQ ID NO: 84) |
| PAX6 | AAC AAC CTG CCT ATG CAA CC | ACT TGG ACG GGA ACT GAC AC |
| | (SEQ ID NO: 85) | (SEQ ID NO: 86) |
| Pitx3 | CCA CCC TAC GAG GAG GTG TA | GGG CGG TGA GAA TAC AGG T |
| | (SEQ ID NO: 87) | (SEQ ID NO: 88) |
| PRNP | GTG GCT ACA TGT TGG GGA GT | GTG AAG TTC TCC CCC TTG GT |
| | (SEQ ID NO: 89) | (SEQ ID NO: 90) |
| S100β | GGC GGC AAA AGG TGA CCA GGA | GCC CTC ATG TCT GCC ACG GG |
| | (SEQ ID NO: 91) | (SEQ ID NO: 92) |
| SERPIN | ACC CGA GTA CCG AGA GGA TT | ACT GTG GGG ACT TCA CAA GG |
| B1B | (SEQ ID NO: 93) | (SEQ ID NO: 94) |
| SHH | AAA AGC TGA CCC CTT TAG CC | TGC ACC TCT GAG TCA TCA GC |
| | (SEQ ID NO: 95) | (SEQ ID NO: 96) |
| SOD3 | GAC CTG GAG ATC TGG ATG GA | GTG GTT GGA GGT GTT CTG CT |
| | (SEQ ID NO: 97) | (SEQ ID NO: 98) |
| SPO1 | TAC CAT GTC GGA GTG GAT CA | ACT CAC CCC ACT CAG TCA CC |
| | (SEQ ID NO: 99) | (SEQ ID NO: 100) |
| SPO2 | AGT GGA GCC AGA CAG CAT TT | CTC CTG CTG CTT CGA TCT CT |
| | (SEQ ID NO: 101) | (SEQ ID NO: 102) |
| TGF-B | ATA CGC CTG AGT GGC TGT CT | TGG GAC TGA TCC CAT TGA TT |
| | (SEQ ID NO: 103) | (SEQ ID NO: 104) |
| Thbs1 | CCA GTT CAA CCAACG TCC TT | TTG CGA ATG CTG TCC TGT AG |
| | (SEQ ID NO: 105) | (SEQ ID NO: 106) |
| TNF-α | AGA TGT GGA ACT GGC AGA GG | CCC ATT TGG GAA CTT CTC CT |
| | (SEQ ID NO: 107) | (SEQ ID NO: 108) |
| TXNIP | GGC ACA CTT ACC TTG CCA AT | ACT AGG GGC AGA TCG AGG AT |
| | (SEQ ID NO: 109) | (SEQ ID NO: 110) |
| Vimentin | AGA TCG ATG TGG ACG TTT CC | CAC ACT GTC TCC GGT ATT CGT |
| | (SEQ ID NO: 111) | (SEQ ID NO: 112) |
| Wnt1 | CTT CGG CAA GAT CGT CAA CC | GCG AAG ATG AAC GCT GTT TCT |
| | (SEQ ID NO: 113) | (SEQ ID NO: 114) |
| Wnt3a | GAA CGC GAC CTG GTC TAC TAC G | GTT AGG TTC GCA GAA GTT GGG T |
| | (SEQ ID NO: 115) | (SEQ ID NO: 116) |
| Wnt4 | GCC ACG CAC TAAAGG AGA AG | GGC CTT AGA CGT CTT GTT CG |
| | (SEQ ID NO: 117) | (SEQ ID NO: 118) |
| Wnt5a | AAT AAC CCT GTT CAG ATG TCA | TAC TGC ATG TGG TCC TGA TA |
| | (SEQ ID NO: 119) | (SEQ ID NO: 120) |
| Wnt7a | CGA GAG CTA GGC TAC GTG CT | CTG AGG GGC TGT CTT ATT GC |
| | (SEQ ID NO: 121) | (SEQ ID NO: 122) |
| YM1 | TCG TGA GAA GCT CAT TGT GG | AAC CCA TAC ATT GCC CTG AA |
| | (SEQ ID NO: 123) | (SEQ ID NO: 124) |

RNA-Seq Analysis

RNA sequencing was carried out in Macrogen (Seoul, Korea). After trimming reads with a quality score less than 20 using FastQC and checking the mismatch ratio using Bowtie, all RNA-seq data were mapped to the mouse reference genome (GRCm38/mm 10) using STAR [Dobin, A., Davis, C. A., Schlesinger, F., Drenkow, J., Zaleski, C., Jha, S., Batut, P., Chaisson, M., and Gingeras, T. R. 2013. STAR: ultrafast universal RNA-seq aligner. Bioinformatics 29:15-21.]. To measure expression levels of all 46,432 annotated genes, 107,631 transcripts, and 12 76,131 protein-coding (mRNA) records in the mouse genome (based on NCBI RefSeq annotations Release 105: February 2015), we counted reads mapped to the exons of genes using Htseq-count and calculated the Fragments Per Kilobase of exon per Million fragments mapped (FPKM) value. Quantile normalization was performed to reduce technical global bias of expression between groups [Bolstad, B. M., Irizarry, R. A., Astrand, M., and Speed, T. P. 2003. A comparison of normalization methods for high density oligonucleotide array data based on variance and bias. Bioinformatics 19:185-193.]. All data have been deposited into GEO database (GEO: 17 GSE106216).

Determination of Intracellular ROS and Glutathione Levels

For measurement of intracellular ROS levels, cells were incubated with 10 μM of 5-(and-6)-chloromethyl-2′,7′-dichlorodihydro-fluorescein diacetate [CM-H2DCF-DA (herein referred to as DCF) (Invitrogen)] for 10 min. The cells were then washed with D-PBS (in mM: 2.68 KCl, 1.47 KH2PO4, 136.89 NaCl, and 8.1 Na2HPO4), and fluorescence and phase-contrast images were taken using an Olympus microscope (IX71, Hicksville, N.Y., USA). Determination of intracellular glutathione levels was requested and carried out by Celltoin™ (Seoul, Korea).

DA Release Assay

The pre-synaptic activity of DA neurons was determined by measuring the levels of DA neurotransmitter released in the differentiated VM-NPC cultures. Media incubated for 24 hrs (differentiation day 12-13) was collected and used to determine the DA level using an ELISA kit (BA E-5300, LDN). In addition, DA release evoked by membrane depolarization was estimated by incubating the cultures (at differentiation day 12) in fresh N2 media in the presence or absence of 56 mM KCl for 30 min. The evoked DA release was calculated by subtracting the DA release without KCl from the DA level with KCl.

Glutamate Uptake

Cells were washed twice in Tissue Buffer (5 mM Tris, 320 mM sucrose, pH 7.4) and exposed to 10 uM glutamate in either Na+-containing Krebs buffer (120 mM NaCl, 25 mM NaHCO3, 5 mM KCl, 2 mM CaCl2, 1 mM KH2PO4, 1 mM MgSO4, and 10% glucose) or Na+-free Krebs (120 mM choline-Cl and 25 mM Tris-HCl) for 10 min at 37° C. Uptake was stopped by placing the cells on ice and washing them twice with Wash Buffer (5 mM Tris/160 mM NaCl, pH 7.4). Cells were collected and homogenized in 100 ul of assay buffer and the amount of glutamate in the cell homogenates was measured using a glutamate assay kit (Abcam, Cambridge, Mass., USA, ab83389). Na+-dependent uptake was determined by subtracting Na+-free counts from total counts in the presence of Na+.

Confirmation of α-Synuclein Aggregation After Co-Culture of Astrocytes with α-Synuclein-Overexpressing DA Neurons

Alpha-synuclein-overexpressing ventral midbrain DA neurons obtained by differentiation of ventral midbrain neural stem cells (VM-NPC) transduced with α-synuclein-overexpressing lentiviruses were cultured by cell culture. Here, α-synuclein-overexpressing DA neurons were co-cultured with (1) astrocytes isolated from the cortex (Ctx-astrocytes) or (2) astrocytes isolated from the ventral midbrain (VM-astrocytes), or with neural stem cells isolated from the cortex (Ctx-NPCs) as the control. In addition, during differentiation, pre-formed fibrils (PFF), that is, an α-synuclein aggregate, was treated to induce α-synuclein aggregation, and 20 days after differentiation, the α-synuclein aggregation was identified by immunocytochemistry using thioflavin S (staining for detection of protein aggregation; Sigma Aldrich) and an α-synuclein antibody and by western blotting, and observed through a change in protein size.

Confirmation of Intercellular Transmission of α-Synuclein Aggregate by Factor Secreted from Astrocytes

Neuroblastoma-derived SH-SY5Y cells (provided by Prof. Sang-Myun Park of Ajou University) were used for overexpression of α-synuclein and were co-cultured with non-overexpressed SH-SY5Y cells, followed by treatment of (1) astrocytes isolated from the cortex (Ctx-astrocytes), (2) astrocytes isolated from the ventral midbrain (VM-astrocytes), and neural stem cells isolated from the cortex (Ctx-NPCs) as the control with conditioned media to confirm α-synuclein transmission through the fluorescence of α-syn-GFP by immunocytochemistry using antibodies.

Confirmation of Intercellular α-Synuclein Aggregation Effect by Factor Secreted from Astrocytes

Alpha-synuclein was overexpressed in SH-SY5Y cells, co-cultured with each of α-syn-GFP and α-syn-mCherry, and Ctx-astrocytes, VM-astrocytes and the control (Ctx-NPCs, which are neural stem cells isolated from the cortex) were treated with conditioned media to confirm α-synuclein aggregation by determining expression levels of GFP and mCherry.

Analysis of Environment Improvement Effect by Human VM Astrocytes

Human VM NPCs and human VM astrocytes were cultured and injected into the left and right hemispheres, respectively, to observe and analyze an environment improvement effect by human astrocytes by immunohistochemistry for an M2 marker. Each type of cells (3 μl, 1.5×10⁵ cells/μl) were injected into each of two sites in the striatum (coordinates in AP, ML and V relative to bregma and dura: [1] 0.07, −0.30, −0.55; [2] −0.10, −0.40, −0.50; incision bar set at 3.5 mm below zero) for 10 minutes under of Zoletil (100 μl/100 g; 50 mg/ml) mixed with of Rompun (100 μl /100 g; 23.32 mg/ml) according to “transplantation and histological procedures” described above. Hereinafter, immunohistochemistry was performed according to “transplantation and histological procedures” described above.

[Results]

Immature and Brain Region-Specific Identities of Astrocytes Cultured from the Cortex and VM of Mouse Pups

The aim of the present invention is to improve NPC transplantation efficacy by utilizing neurotrophic actions of astrocytes. Based on brain region-dependent diversity of astrocytes and their potential region-specific roles, we cultured astrocytes from two brain regions including the classical non-dopaminergic brain region cortex (Ctx) and dopaminergic ventral midbrain (VM).

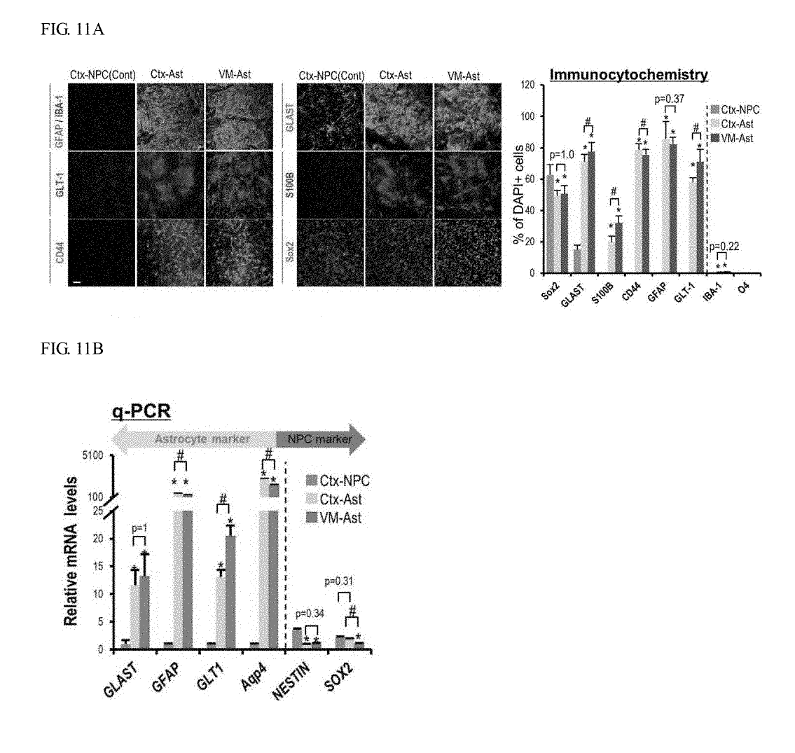

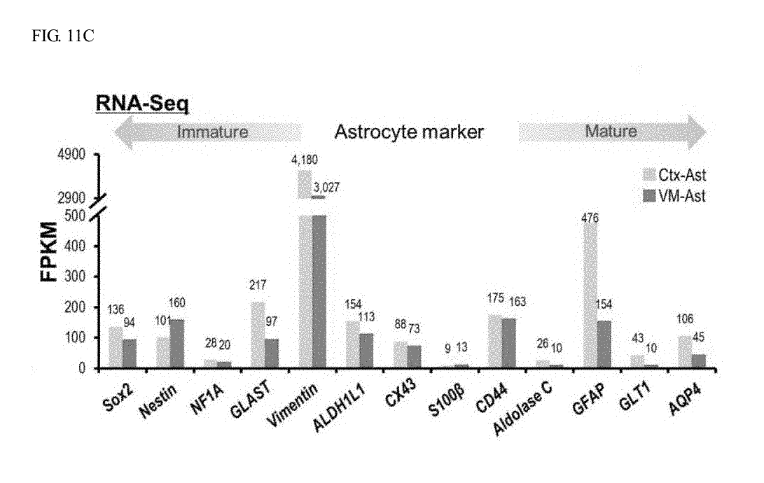

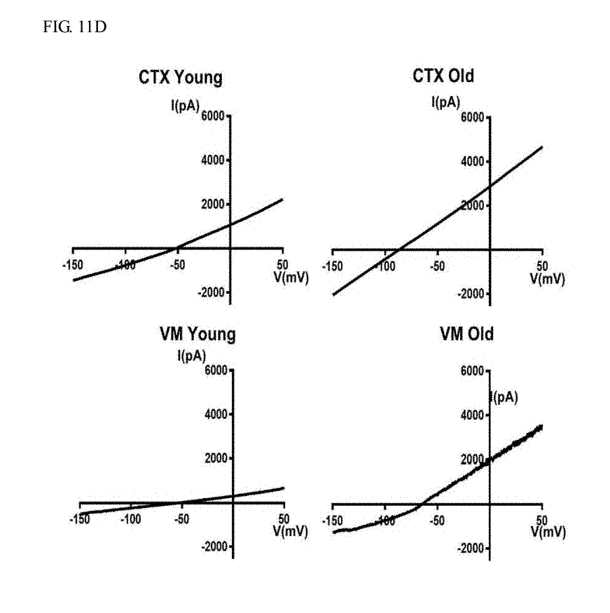

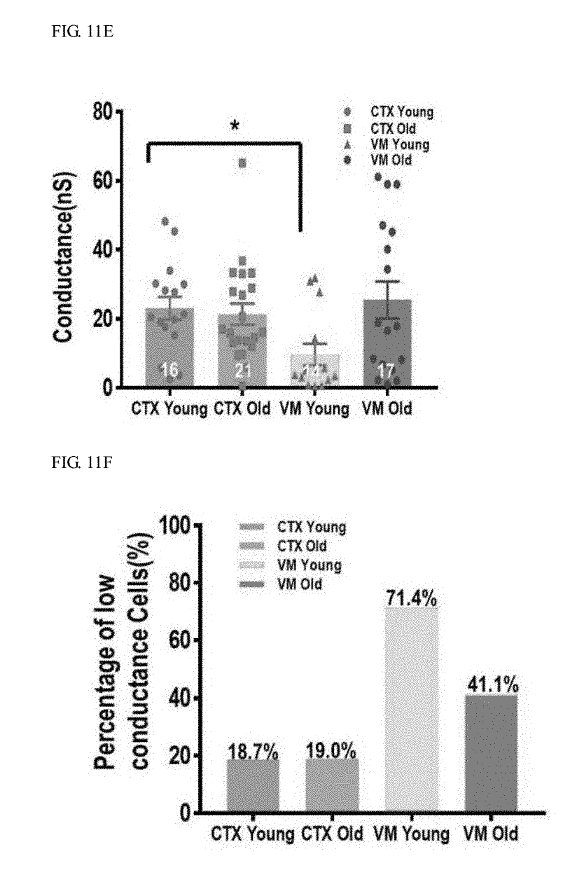

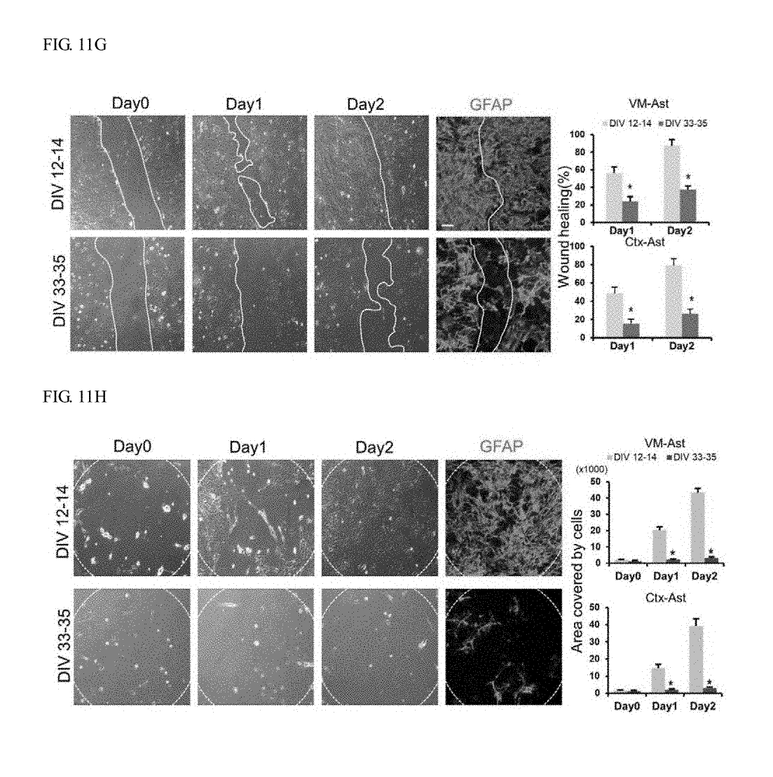

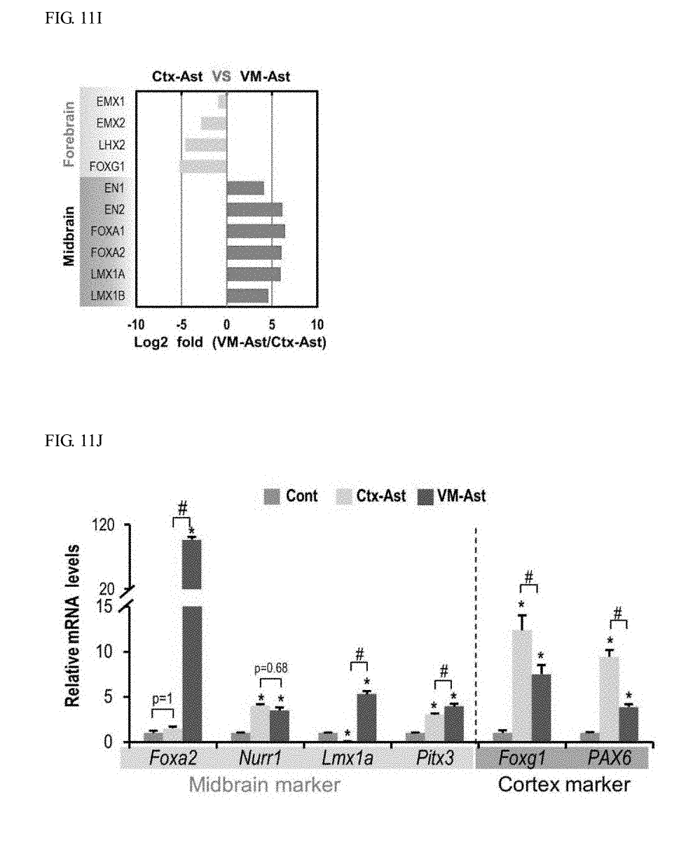

Along with high mRNA expression of astrocytic markers (FIG. 11B), immunocytochemical analyses revealed that major cell populations in the Ctx- and VM-astroglial cultures at days in vitro (DIV) 14 were positive for GFAP (85% and 82%), CD44 (79% and 76%), GLT-1 (58% and 71%), and GLAST protein (71% and 77%), respectively (FIG. 11A), except S1001β(20% and 32%), a soluble protein associated with harmful reactivation of astrocytes. A few (0.24±0.35% in Ctx-Ast and 0.43±0.39% in VM-Ast) and none of the cells in these cultures were Iba1+ microglia and O4+ oligodendrocyte, respectively. By contrast, none or a few cells were positive for the astroglial markers in the NPC cultures derived from embryonic cortices (Ctx-NPCs) used as a control (FIG. 11A).

Along with abundant cells immunoreactive for the immature astrocytic markers GLAST and Sox2, the expression levels of the immature astrocytic genes (Sox2, Nestin, GLAST, and Vimentin) in the RNA-sequencing (RNA-seq) data were high (fragments per kilobase of exons per million reads (FPKM): 94-4180, FIG. 11C), indicating that the astrocytes cultured in this study had immature properties. Previous studies have reported that astrocytes in culture were electrophysiologically immature compared to those astrocytes in vivo [Kressin, K., Kuprijanova, E., Jabs, R., Seifert, G., and Steinhauser, C. 1995. Developmental regulation of Na+ and K+ conductances in glial cells of mouse hippocampal brain slices. Glia 15:173-187. Hwang, E. M., Kim, E., Yarishkin, O., Woo, D. H., Han, K. S., Park, N., Bae, Y., Woo, J., Kim, D., Park, M., et al. 2014. A disulphide-linked heterodimer of TWIK-1 and TREK-1 mediates passive conductance in astrocytes. Nat Commun 5:3227]. Consistently, in patch-clamp analyses, astrocytes cultured from cortices in this study (DIV14) exhibited current (2577±401.1 pA, n=16) and conductance (23.08±3.306 nS, n=16), which were significantly lower than those detected in cortical slices in vivo (40 nS).

Interestingly, the current and conductance values of astrocytes cultured from VM at DIV14 were further lower compared to the cultured Ctx-astrocytes at DIV14, and those of VM-astrocytes increased after a longer culture period (at DIV35)(FIG. 11D and FIG. 11E). Each group of astrocytes had a population with conductance lower than 10 nS. The percentage of low conductance cells was the highest in VM-astrocyte cultures at DIV14(young) and decreased during maturation in VM at DIV35(old), while the percentages were similar in Ctx DIV14 and Ctx DIV35 (FIG. 11F).

Of note, it has been shown that transplanted immature—but not mature—astrocytes exert neurotrophic support in the injured CNS. In addition to a wound healing capacity superior to astrocytes at DIV33-35 (FIG. 11G) in a scratch injury model, astrocytes at DIV12-14 showed greater capacity growing on high levels of chondroitin sulfate proteoglycans (CSPGs)(FIG. 11H) in an in vitro model recreating the strongly inhibitory CSPG gradient observed in the glial scar in vivo. These findings collectively indicated that cultured astrocytes in this study, especially those at DIV12-14, were immature and capable of exerting regenerative capacities.

Based on these findings, the following experiments were done using astrocyte cultures at DIV7-14, unless otherwise noted.

Positional identity, a fundamental organizing principle governing the generation of neuronal subtype diversity, is relevant to astrocyte diversification. Consistently, Ctx-astrocytes had high levels of expression of cortical region-specific genes (Emx2, Lhx2, FoxG1, and Pax6), whereas expression of the midbrain-specific makers (Foxa1/2, Lmx1a/b, and Ent1/2) was enriched in VM-astrocyte cultures (FIG. 11I, FIG. 11J). While endogenous expression of Foxa2 was exclusively enriched in VM-derived astrocytes, expression of Nurr1, another major gene of interest in this study, and its downstream target gene Pitx3, was indistinguishable between Ctx- and VM-derived astrocyte cultures (FIG. 11I, FIG. 11J).

This finding is consistent with the detection of Nurr1 expression not only in the midbrain, but also in cortical layer VI and hippocampus. These findings collectively verify immature astrocytic- and brain region-specific identities of the astrocyte cultures used in this study.

VM-Astrocytes Promote the Differentiation of NPCs into mDA Neurons with Enhanced Neuronal Maturity, Expression of Midbrain-Specific Markers, and Resistance Against a Toxic Stimulus

In order to attain therapeutic efficacy by NPC transplantation in PD, grafted NPCs must undergo differentiation into mDA neurons, neuronal maturation with synapse formation, and survival of the grafted mDA neurons in the hostile environment of the grafted brain.

We first assessed this series of the cellular events in the absence or presence of Ctx- or VM-astrocytes in vitro.

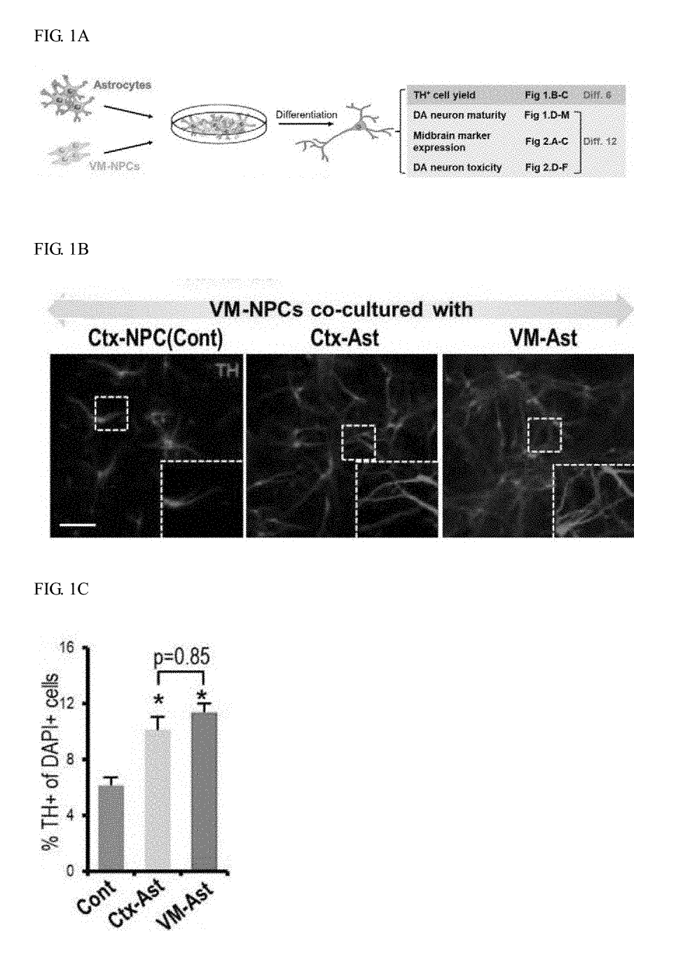

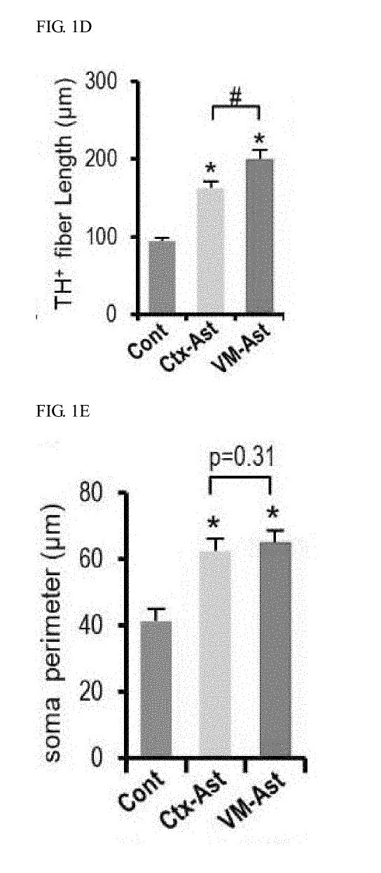

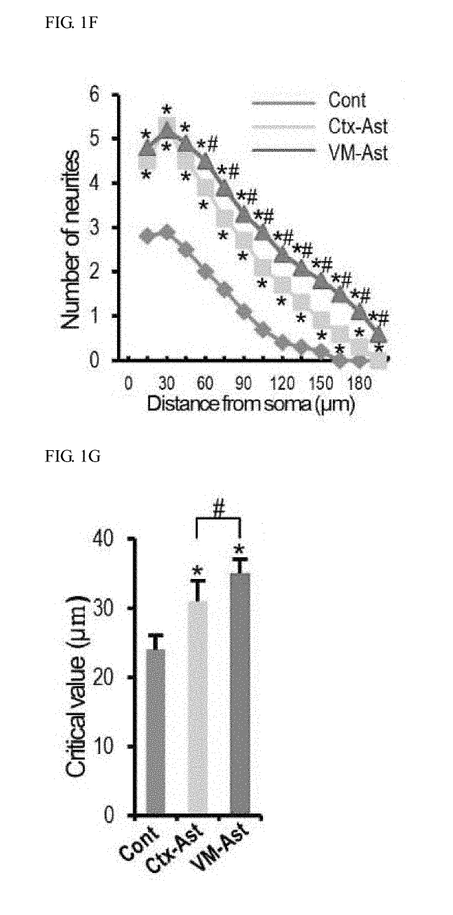

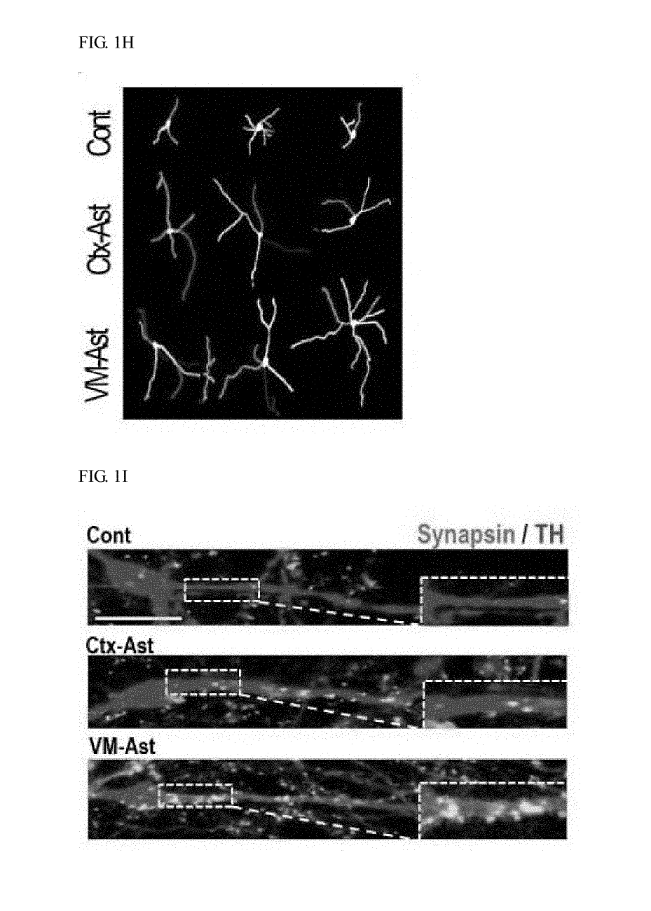

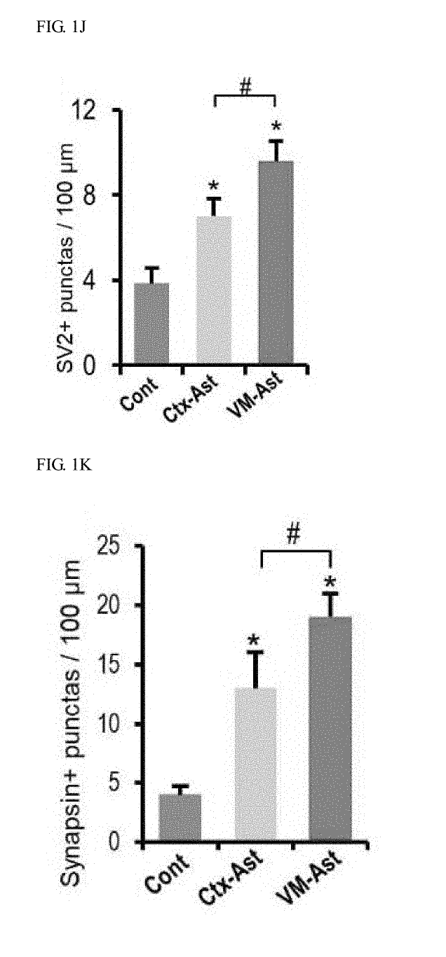

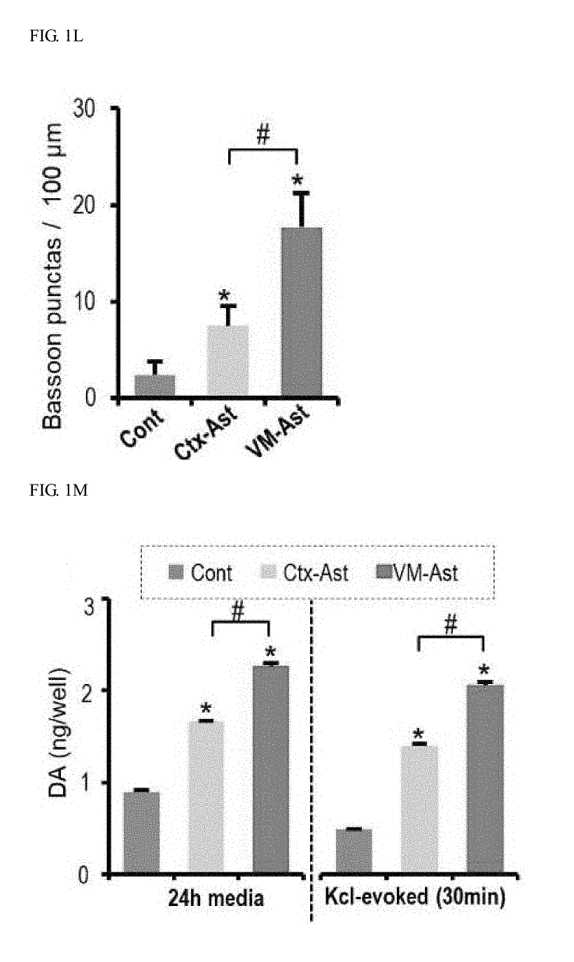

To this end, NPCs isolated from rodent embryonic VMs (mouse at E10.5 or rat at E12) were expanded in vitro for 4 days, harvested and mixed with the Ctx-astrocytes or VM-astrocytes at a cell number ratio of 2:1 VM-NPCs to astrocytes. The mixed cells were plated and differentiation of VM-NPCs was directly induced in serum-free N2 medium (schematized in FIG. 1A).

Since cell differentiation is affected by cell density, VM-NPCs in the control cultures were mixed with non-dopaminergic Ctx-NPCs and we determined that cell density (confluence) among the tested groups was not largely different during the assays.

When DA neuron differentiation was assessed at differentiation day 6 by the number of cells positive for tyrosine hydroxylase (TH), a key enzyme in DA biosynthesis, the number of TH+ cells was significantly greater in the cultures mixed with astrocytes compared to control cultures mixed with Ctx-NPCs (FIG. 1B, FIG. 1C). There was no significant difference in the TH+ cell yields promoted by co-culturing with VM-astrocytes compared to co-culturing with Ctx-astrocytes (FIG. 1C). TH+ DA neurons differentiated in the presence of astrocytes exhibited more mature neuronal morphology than control cultures (FIG. 1B, insets), which was quantified by neurite length (FIG. 1D) and soma size (FIG. 1E) of the TH+ cells. The morphometric maturity of TH+ DA neurons was further assessed based on the complexity of neurite outgrowth using the Sholl test (FIG. 1F, FIG. 1G). In contrast to the lack of difference in TH+ cell yields promoted by Ctx-vs. VM-astrocytes, TH+ DA neurons co-cultured with VM-astrocytes exhibited significantly greater neurite outgrowths than those co-cultured with Ctx-astrocytes (FIG. 1B, FIG. 1D, FIG. 1F-FIG. 1H).

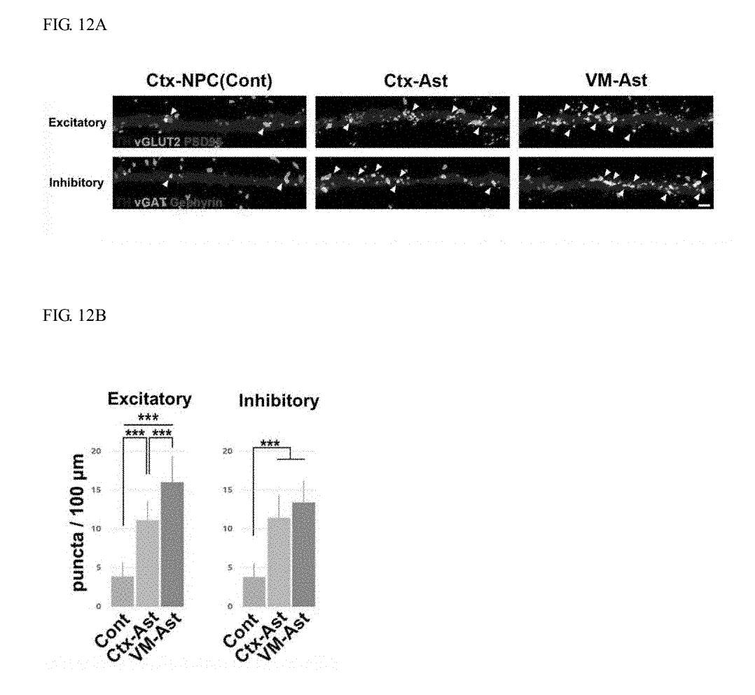

In images captured by a confocal microscope, puncta positive for the synaptic vesicle-specific markers SV2, synapsin, and Bassoon were more abundantly localized in the neurites of the TH+ DA neurons differentiated with astrocytes, and the puncta densities were greatest in those co-cultured with VM-astrocytes (FIG. 1I-FIG. 1L). The types of synapses formed were further analyzed by using neurotransmitter specific pre- and post-synaptic markers. Nigral mDA neurons in the midbrain physiologically receive glutamatergic excitatory and GABAergic inhibitory synaptic inputs from several regions of the brain. Excitatory postsynaptic density protein 95 (PSD95) clusters were found on dendritic shafts of TH+ DA neuronal cells and many of them were juxtaposed with the vesicular glutamate transporter 2 (vGlut2) puncta, which indicates the presence of aspiny glutamatergic shaft synapses in TH+DA neurons (FIG. 12A). We found that vGlut2+/PSD95+ clusters were more abundantly detected in TH+ DA neurons co-cultured with astrocytes than those in control cultures (FIG. 12A, FIG. 12B).

Along with significantly more vGlut2+/PSD95+ clusters on TH+ cells co-cultured with VM-astrocytes than those with Ctx-astrocytes, the glutamatergic synapses on spine-like structures were also found only with VM-astrocytes, further indicating more mature neuronal morphology with VM-astrocytes. GABAergic inhibitory (vGAT+/Gephyrin+) synaptic puncta were also more abundantly localized in the fibers of TH+ DA neurons cultured with astrocytes (FIG. 12A, FIG. 12B).

We further observed that DA release, especially when evoked by KCl-induced depolarization, was significantly promoted in the cultures differentiated with astrocytes; the pre-synaptic DA release promoted by astrocytes was greater in the cultures with VM-astrocytes than in the cultures with Ctx-astrocytes (FIG. 1M). Midbrain-type DA (mDA) neurons are specified based on the expression of several midbrain-specific genes such as Nurr1, Foxa2, Lmx1a/b, Pitx3, and Ent1/2. As these midbrain markers play critical roles in DA phenotype maintenance, survival and function, their expression is critical in the preparation of mDA neurons for therapeutic purposes.

However, we and others have recently shown that the expression of midbrain-specific markers in mDA neurons is largely affected by in vitro and in vivo environments, and thus easily lost during passages, long after differentiation in vitro and after transplantation in vivo.

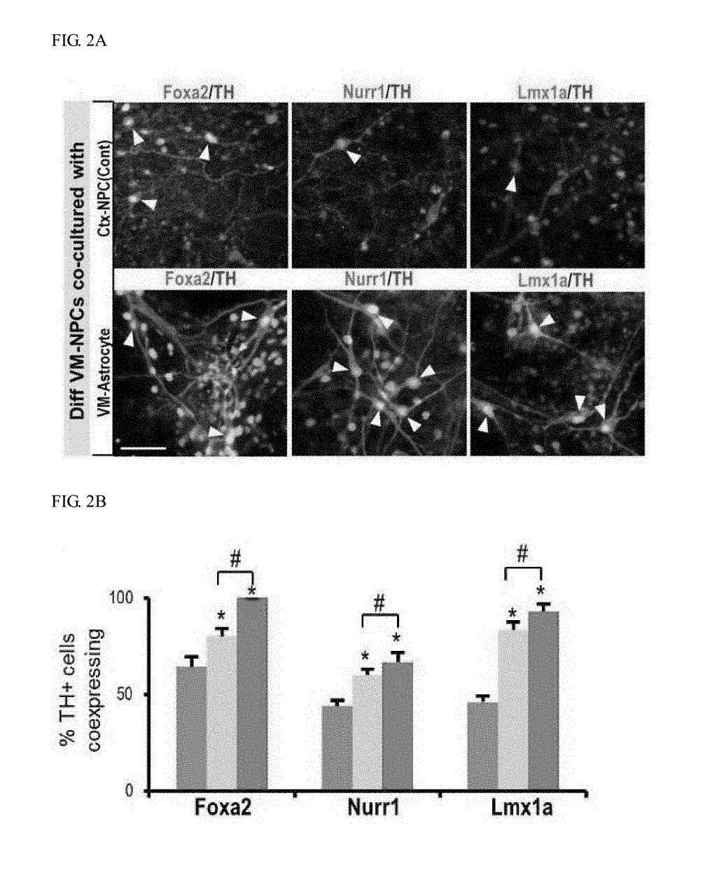

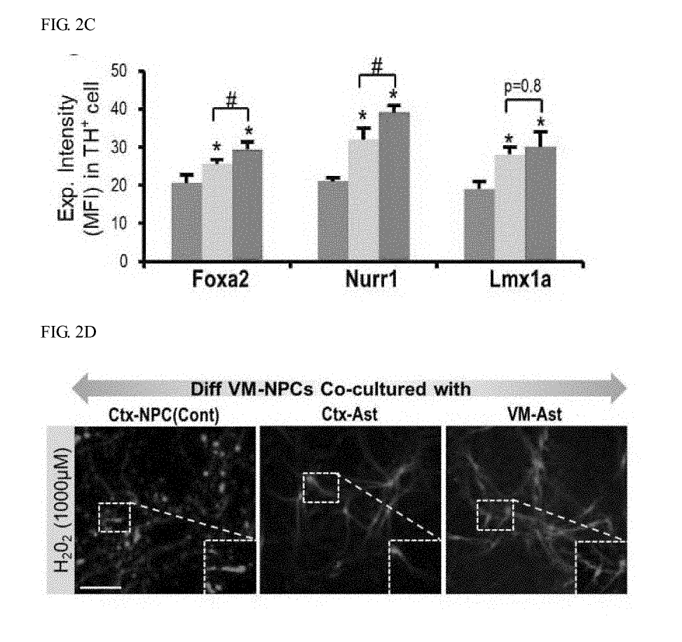

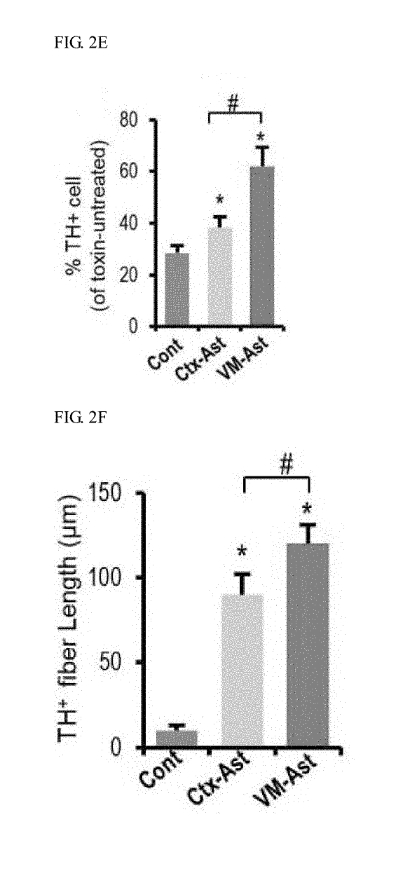

Indeed, expression of Foxa2, Nurr1, and Lmx1a was detected only in 64, 44, and 46% of TH+ cells at day 12 of differentiation, respectively (FIG. 2A, FIG. 2B). The percentage of TH+ DA neurons expressing the midbrain markers (FIG. 2B) and their expression levels in individual TH+ cells (estimated by mean fluorescence intensity [MFI] in the nucleus of TH+ cells (FIG. 2C) were significantly greater in the cultures mixed with astrocytes. The co-culture with VM-astrocytes had the greatest effect with almost all TH+ cells co-expressing the midbrain-specific markers, further supporting the functional superiority of VM-astrocytes over Ctx-astrocytes in their midbrain dopaminotrophic actions. When the differentiated cultures were exposed to the ROS producing agent H₂O₂ (1000 μM for 10 hr), only 15% of the toxin-untreated TH+ DA neurons survived in the control cultures mixed with Ctx-NPCs (FIG. 2D, FIG. 2E). As expected, remarkably lower proportions of DA neurons died after the toxic stimulus in the presence of astrocytes.

In addition, TH+ cells in the cultures mixed with astrocytes survived the toxin treatment and displayed healthy neuronal morphology with extensive neurite outgrowths, while most of the surviving TH+ cells in the control cultures had blunted or fragmented neurites (FIG. 2D, insets), a neuronal aging and degenerative phenotype. This finding was further supported by quantification of the fiber length in the surviving TH+ cells (FIG. 2F). Again, VM-astrocytes exerted significantly greater protective effects on mDA neurons than Ctx-astrocytes (FIG. 2D-FIG. 2F).

Collectively, these findings suggest that cultured astrocytes facilitate VM-NPC differentiation towards authentic midbrain-type DA neurons which are morphologically, synaptically, and functionally mature and are resistant to toxic insults. In addition, our data show that astrocytes of VM-origin are superior to astrocytes from the Ctx in terms of their trophic actions on mDA neuron differentiation and survival.

Paracrine Manner of the Observed Astrocytic Actions

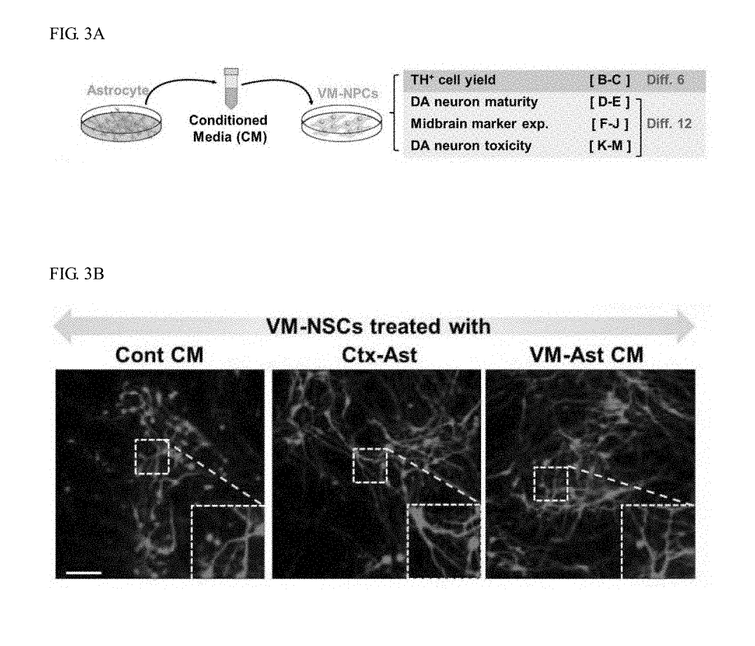

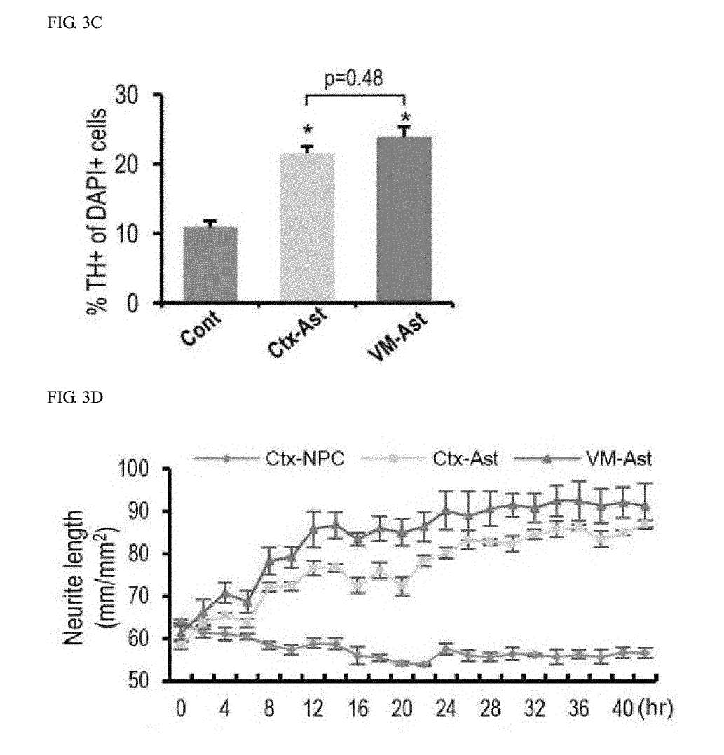

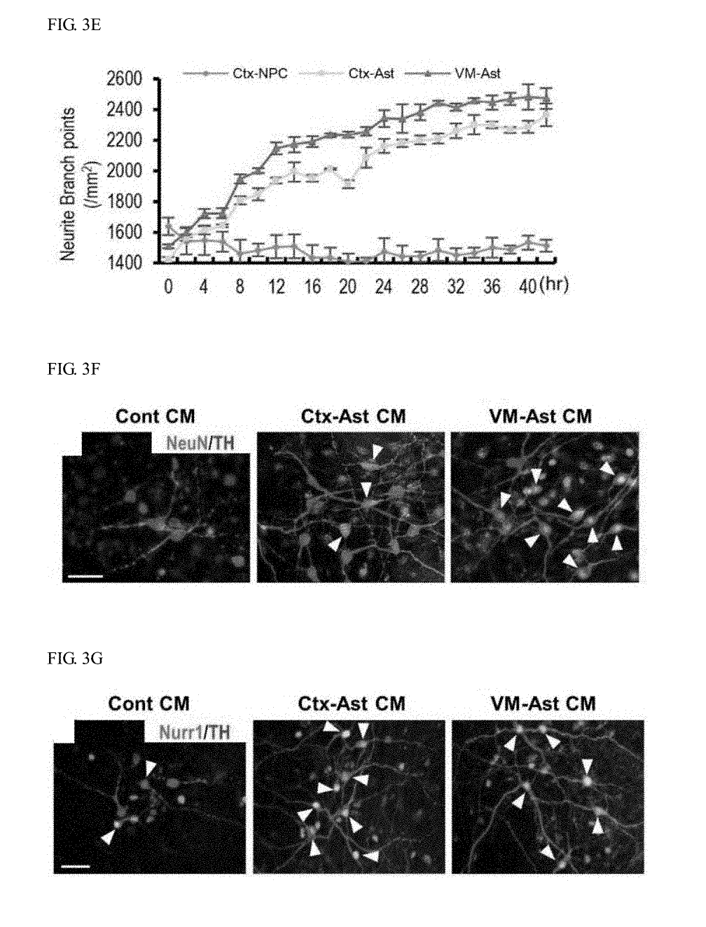

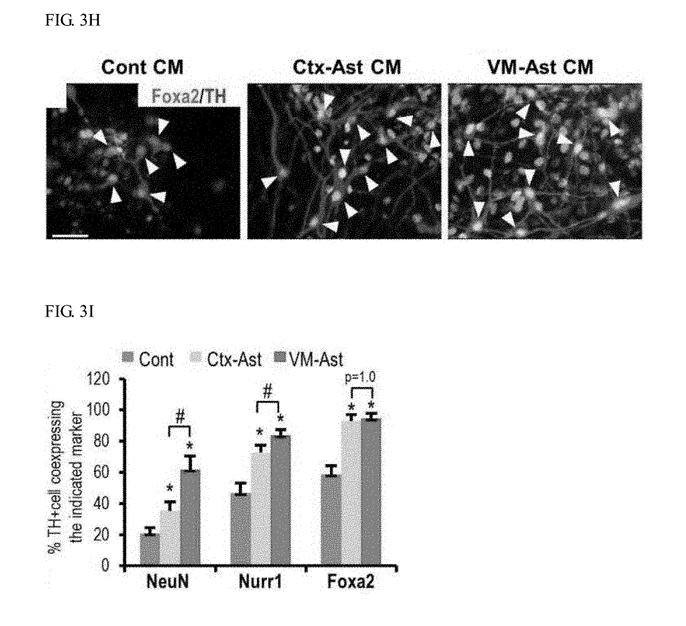

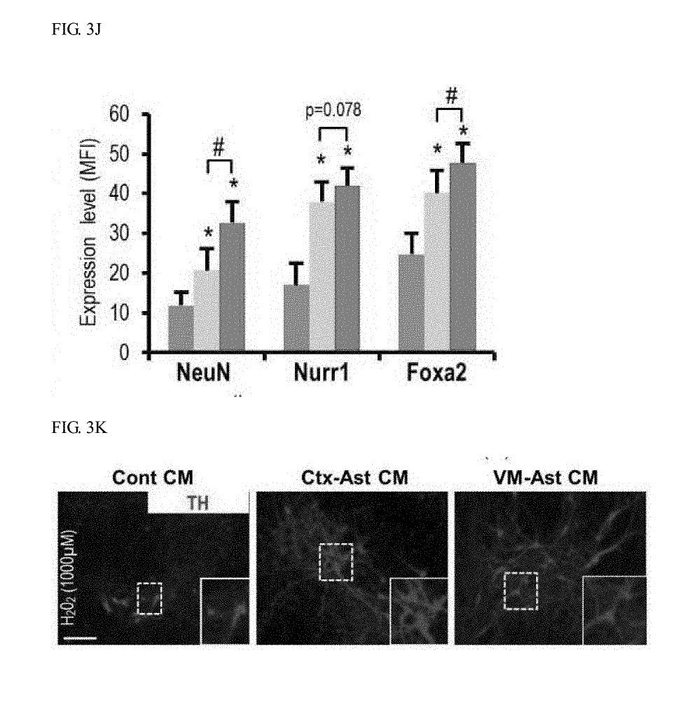

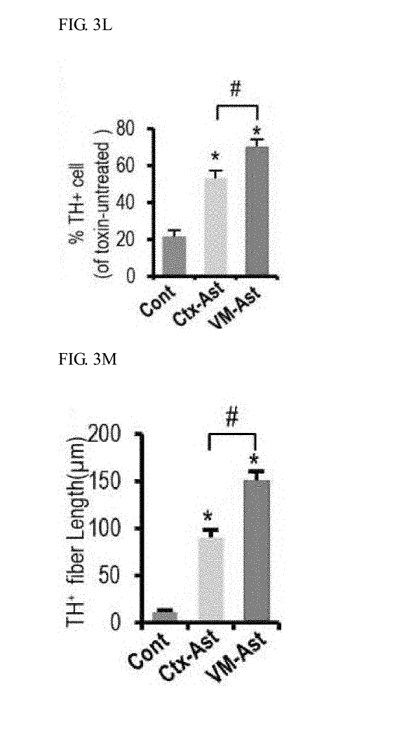

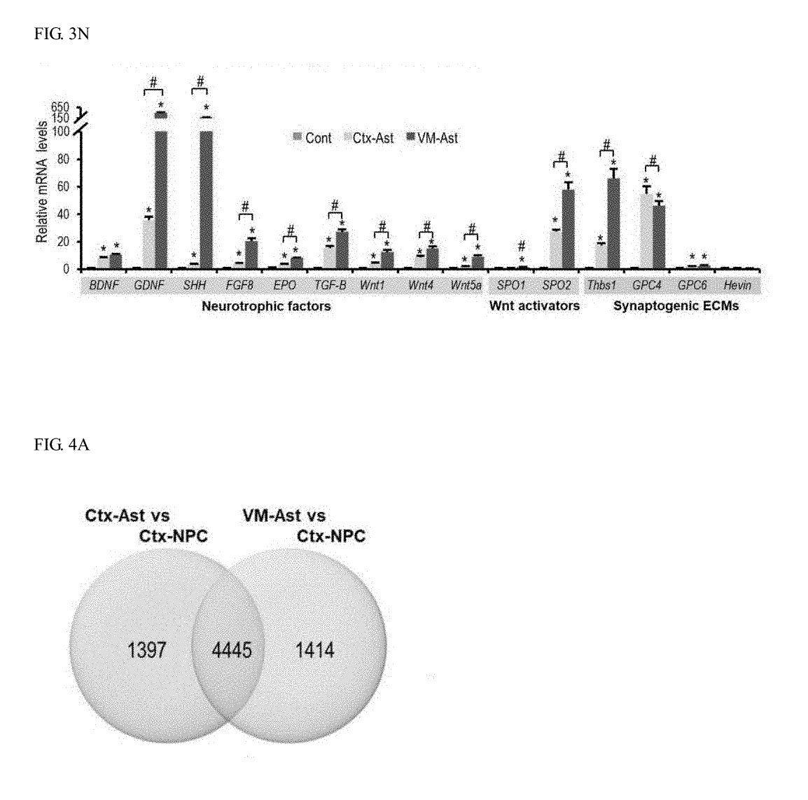

The observed astrocytic actions could be mediated by factors released from the astrocytes and/or cell-cell contact signaling. To test the possibility of paracrine effects, the medium was conditioned in the astrocyte cultures (or differentiated Ctx-NPC cultures as the control) for 2 days, and the conditioned medium (CM) was added to undifferentiated VM-NPC cultures (FIG. 3A). The astrocyte-CM more efficiently promoted VM-NPC differentiation into DA neurons compared to the control-CM as estimated by TH+ cell yields 6 days after differentiation (FIG. 3B, FIG. 3C). In addition, fiber outgrowth (FIG. 3D) and branching (FIG. 3E) during the 42 hrs of CM treatment increased in the differentiating cultures treated with astrocyte-CM compared to the control-CM, with the greatest increase by VM-astrocyte-CM treatment. Along with the morphologic maturation effects, similar patterns of the astrocyte-CM effects were manifested in the expression of NeuN, a mature neuron marker, in TH+ DA neuronal cells (FIG. 3F, FIG. 3I, FIG. 3J). Expression of the midbrain-specific factors Nurr1 and Foxa2 (FIG. 3G FIG. 3H, FIG. 3I, FIG. 3J), and TH+ cell survival with H₂O₂ toxin treatment (FIG. 3K-FIG. 3M) were greatly improved by VM-astrocyte-CM treatment, compared with cultures treated with control-CM or Ctx-astrocyte-CM. These findings collectively indicate that the astrocyte functions observed in FIG. 1A-FIG. 1M and FIG. 2A-FIG. 2F were at least in part mediated by paracrine factors secreted from the astrocytes.

We next sought to identify the paracrine factors responsible for the astrocyte-mediated neurotrophic actions. Consistent with neurotrophic factor secretion from astroglial cells, cultured astrocytes expressed elevated levels of mRNAs for various neurotrophic factors (FIG. 3N).

Of note, the expression of glial cell-derived neurotrophic factor (GDNF), sonic hedgehog (SHH), and fibroblast growth factor 8 (FGF8) was greatly elevated in VM-astrocytes compared to Ctx-astrocytes or control Ctx-NPCs. GDNF has physiologic neurotrophic roles specific for mDA neurons, and SHH, by establishing an auto-regulatory loop with Foxa2, is one of the most important factors in mDA neuron development and survival. The cooperative actions of FGF8 and SHH are critical for mDA neuron development and thus this cytokine combination is commonly being used for in vitro mDA neuron pattering from stem cells. In addition, crucial roles for Wnt/β-catenin signaling from mDA neurogenesis to regeneration, and secretion of Wnt1 and Wnt5a proteins from VM-astrocytes have been reported.

Consistently, cultured VM-astrocytes abundantly expressed not only Wnt cytokines (Wnt1, 4, 5a), but also spondin-2 (SPO-2) (FIG. 3N), a secreted protein of the R-spondin family, which activates Wnt/β-catenin signaling by preventing clearance of the Frizzled—LRP Wnt receptor complex.

In addition, cultured astrocytes exhibited an elevated expression of thrombospondin-1 (THBS-1), Glypicans (GPC) 4 and 6, and secretory extracellular matrix (ECM) proteins which promote synapse formation (53, 54); THBS-1 mRNA levels were higher in VM-astrocyte cultures than in Ctx-astrocytes, suggesting that these factors were responsible for the synaptic maturation of DA neurons in the presence of VM-astrocytes (FIG. 1I-FIG. 1L and FIG. 12A-FIG. 12B).

Differentially Expressed Genes Among the Differentiated NPCs, Ctx-Astrocytes, and VM-Astrocytes

To gain further molecular insight into the observed astrocyte functions, we performed RNA-sequencing (RNA-seq) analysis of the differentiated Ctx-NPCs (control), Ctx-astrocytes and VM-astrocytes used in the co-culture and CM experiments.

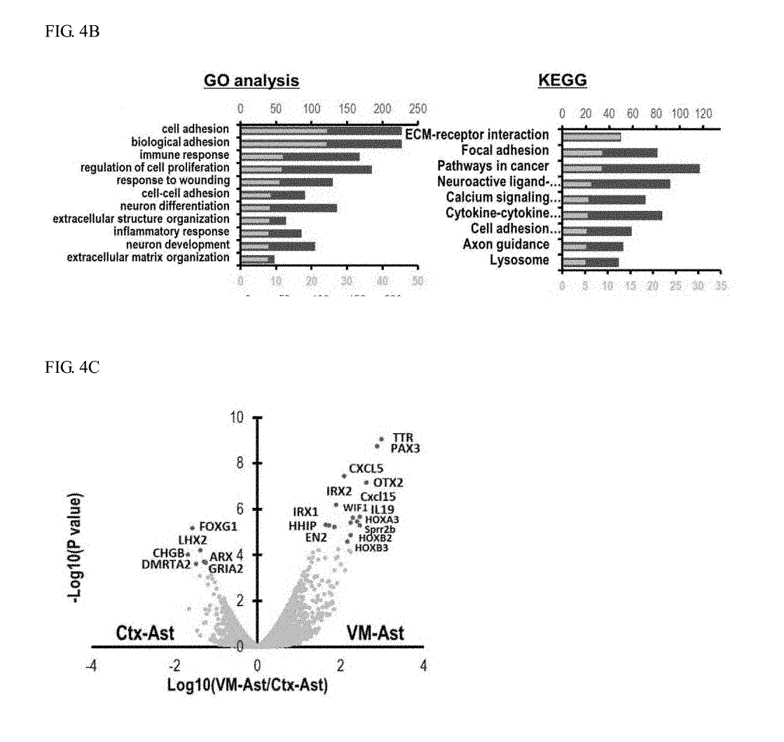

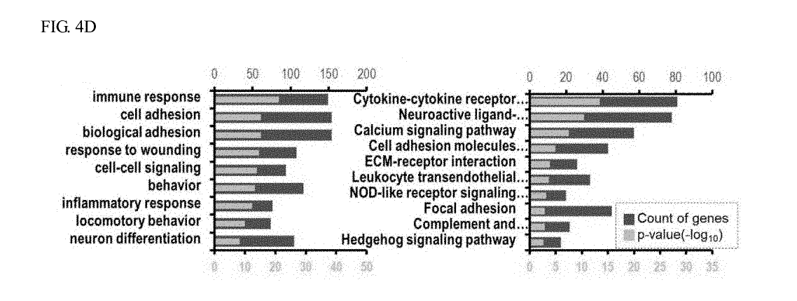

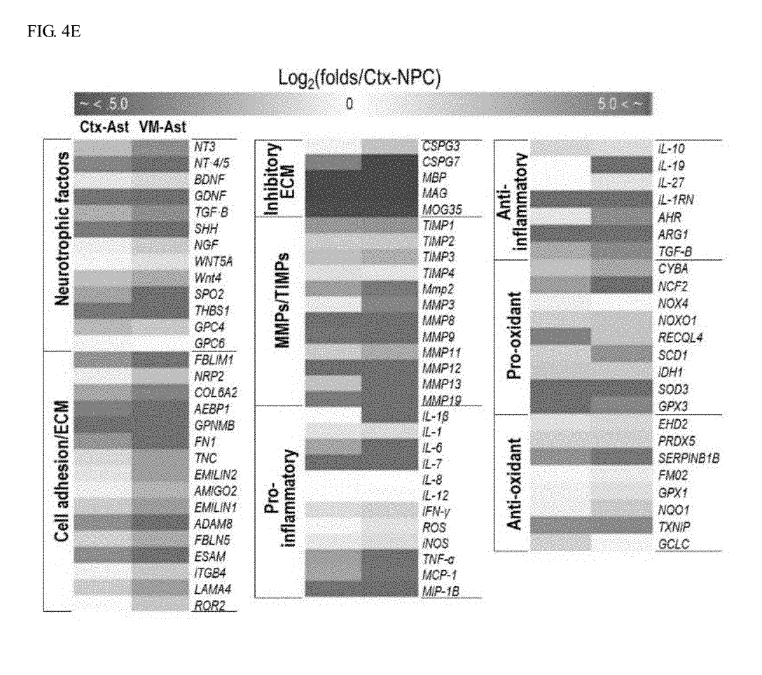

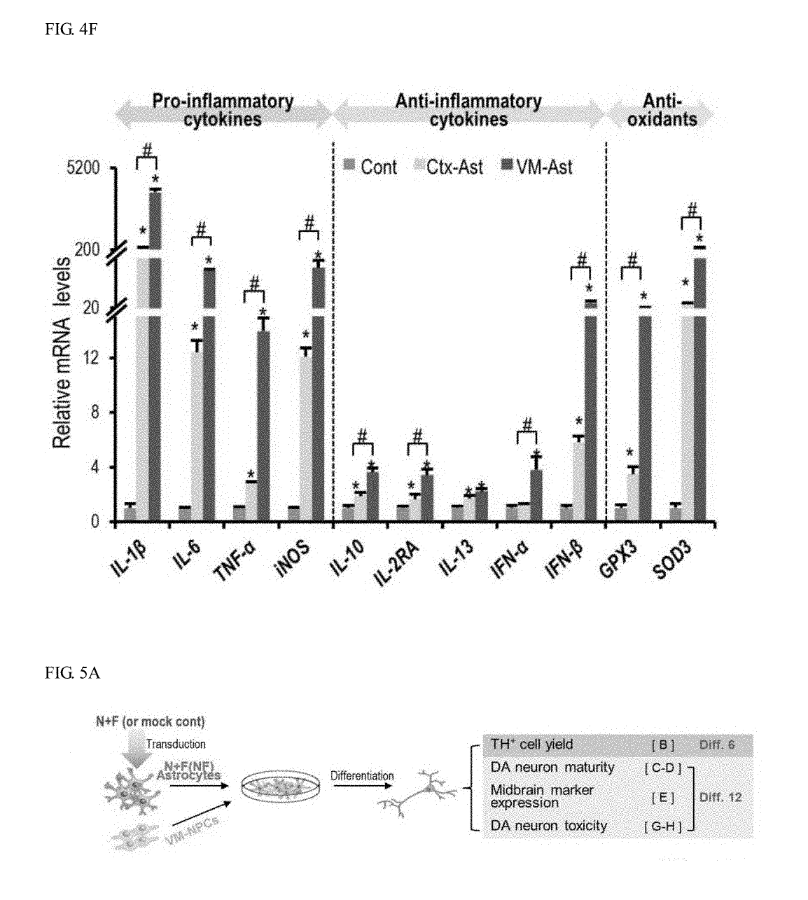

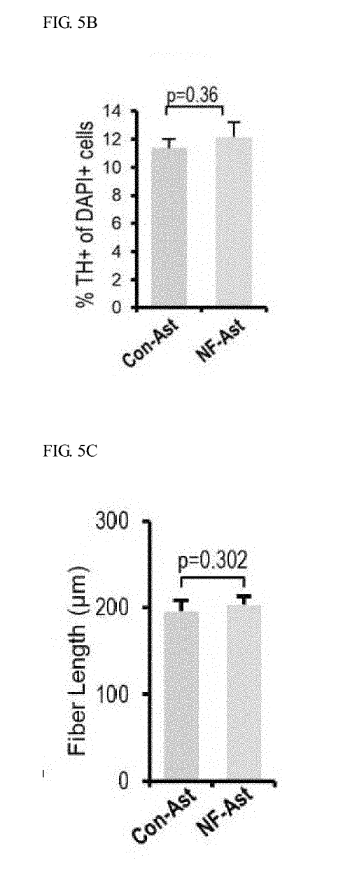

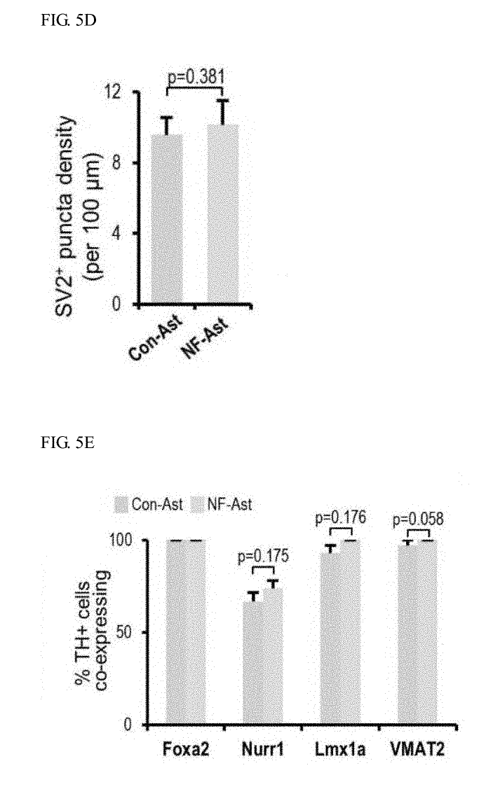

The genes that are differentially expressed (DEGs) in Ctx-astrocytes compared to differentiated Ctx-NPCs (FPKM>1, log 2>1) significantly overlapped with DEGs in VM-astrocytes compared to differentiated Ctx-NPCs (‘N-1’, Chi square=9059.4, df=1, P<2.2e-16, FIG. 4A). In a gene set analysis, the overlapped genes (hereafter, referred to as ‘common astrocytic genes’) were enriched in the gene ontologies associated with ‘cell adhesion/extracellular matrix (ECM)’ ‘immune/inflammatory response’, and ‘neuron differentiation’ (FIG. 4B). Surprisingly, the top ranked gene ontologies for DEGs between Ctx-astrocytes and VM-astrocytes also fell within similar gene categories (FIG. 4C, FIG. 4D).

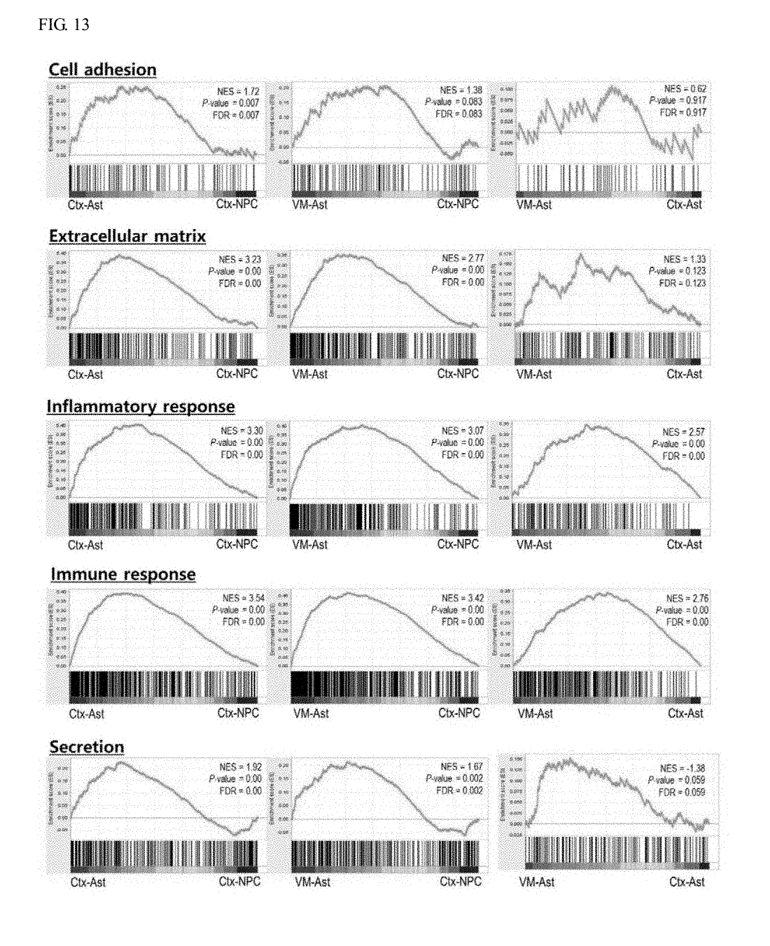

The gene sets enriched in cultured astrocytes (vs control NPCs) and VM-astrocytes (vs Ctx-astrocytes) were further confirmed in gene set enrichment analysis (GSEA) (FIG. 13). Specifically, the differential expression patterns for the neurotrophic factors determined by q-PCR (FIG. 3N) were similarly replicated in the RNA-seq analyses (FIG. 4E), indicating fidelity of the RNA-seq analysis and further confirming that these neurotrophic factors are responsible for the observed trophic effects of astrocytes.

Cell adhesion/ECM that was annotated to one of the top ranked ontologies is of prime interest due to the importance of cell-to-cell and cell-to-ECM contacts in stem cell behaviors and regenerative processes in damaged tissues. The heat-map for the cell adhesion/ECM molecules exhibiting increased levels of expression in VM-astrocytes (compared to Ctx-astrocytes and/or control NPCs) is shown in FIG. 4E. Among these genes, the upregulated expression of fibronectin 1 and Collagen IV in astrocytes (VM-astrocytes) seems to be of note based on a recent study reporting that successful engraftment of DA neurons requires cell-to-ECM adhesion through the interaction between Integrin and fibronectin in an animal model of PD.

In addition, tenascin, another upregulated gene in VM-astrocytes, has been reported to augment grafted DA neuron attachment and survival after brain injury and transplantation.

Chondroitin sulfate proteoglycans (CSPGs) inhibit axonal regeneration and neurogenesis. Notably, the expression of Neurocan (CSPG3) and Brevican (CSPG7) was greatly down-regulated in the VM-astrocyte cultures compared to Ctx-astrocyte and control NPC cultures (FIG. 4E). In addition, the cultured Ctx- and VM-astrocytes commonly showed a remarkable reduction in the expression of myelin-basic protein (MBP) and myelin-associated protein (MAG), that inhibits neurite outgrowth, and myelin oligodendrocyte glycoprotein (MOG35-55), a peptide causing neuro-inflammation

The enrichment of DEGs in the category of immune/inflammatory response in astrocytes compared to control NPCs is not surprising considering that astrocytes are commonly believed to be a cell-type mediating brain inflammation.

Expression of pro-inflammatory cytokine mRNAs were elevated in astrocyte cultures, and interestingly levels were higher in VM-astrocytes than Ctx-astrocytes (FIG. 4E and FIG. 13).

Pro-inflammatory cytokines can trigger cellular defense mechanisms and are frequently linked to enhanced neuronal differentiation and survival (63-66). Despite these positive aspects, the common idea is that pro-inflammatory cytokines establish a cytotoxic inflammatory milieu. Along with the pro-inflammatory gene expression increase, gene expression of anti-inflammatory and neurotrophic glial markers also increased in the astrocyte cultures (FIG. 4E).

For example, the gene with the top ranked expression enriched in the VM-astrocyte cultures was IL-19, an anti-inflammatory cytokine from the IL-10 family. The mRNA expression levels of the other anti-inflammatory markers arginase 1 (Arg1, an enzyme inhibiting NO biosynthesis) and IL-1 receptor antagonist (IL1RN) were much higher in the cultured VM-astrocytes than in Ctx-astrocyte and control NPC cultures: FPKMs of ARG1 were 49.9, 5.5, and 0.03 and FPKMs of IL1RN were 14.0, 1.4, and 0.04 for VM-astrocytes, Ctx-astrocytes, and control NPCs, respectively (FIG. 4E). The expression of the anti-inflammatory cytokines IL-10 and IL-27 was the highest and only detected in the VM-astrocyte cultures, although they were excluded from the gene enrichment analysis due to low FPKM values (<1). The type I interferons (IFN) α and β exert anti-inflammatory and neuroprotective activities against mDA neuronal degeneration. It has recently been reported that anti-inflammatory actions of type I IFNs are mediated via the aryl hydrocarbon receptor (AHR) in astrocytes.

Of note, along with the increase in type I IFN expression in qPCR analyses (FIG. 4F), the AHR expression level in VM-astrocyte cultures was also greatly unregulated (FPKM: 51.34 [VM-astrocyte], 6.21 [Ctx-astrocyte], and 3.21 [diff. NPC]) (FIG. 4E), indicating that the expression of these anti-inflammatory cytokines/receptors contributes to improved cell survival by mitigating pro-inflammatory cytotoxic effects in the cultures co-cultured with VM-astrocytes.

The increase in expression levels of pro- and anti-inflammatory genes was further confirmed by qPCR analyses (FIG. 4F).

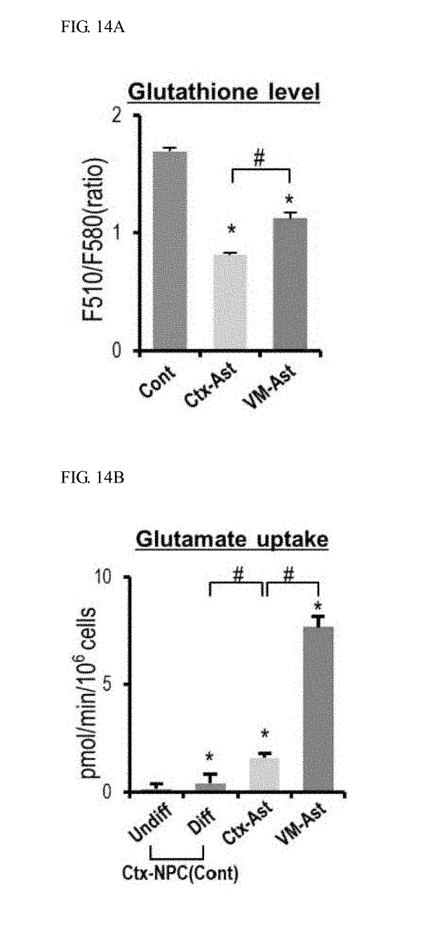

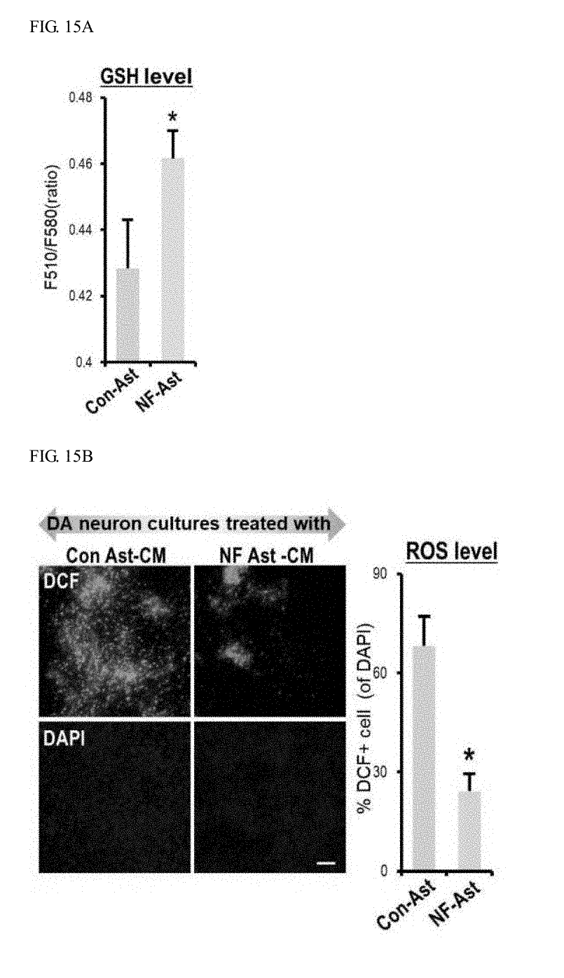

The expression of multiple antioxidant genes was upregulated in the cultured astrocytes compared to control NPCs (FIG. 4E). However, ROS scavenging activity, as estimated by intracellular glutathione levels, was relatively lower in the cultured astrocytes than the control cultures (FIG. 14A).

Another neuroprotective mechanism of astrocytes occurs via clearance of glutamate-induced toxicity (71). Along with enriched expression of the glutamate transporters GLAST and GLT-1 in cultured astrocytes (FIG. 11B, FIG. 11C), glutamate uptake activities were greater in cultured astrocytes than in the control NPCs (FIG. 14B). Notably, the much greater glutamate uptake in cultured VM-astrocytes compared to Ctx-astrocytes suggests that the enriched glutamate clearance activity contributes to the observed neuroprotective actions mediated by astrocytes, especially VM-astrocytes.

Forced Nurr1+Foxa2 Expression Further Potentiates the Neuroprotective Actions of VM-Astrocytes

Next, we investigated if forced Nurr1+Foxa2 expression in VM-astrocytes further promotes the astrocyte-mediated dopaminotrophic actions.

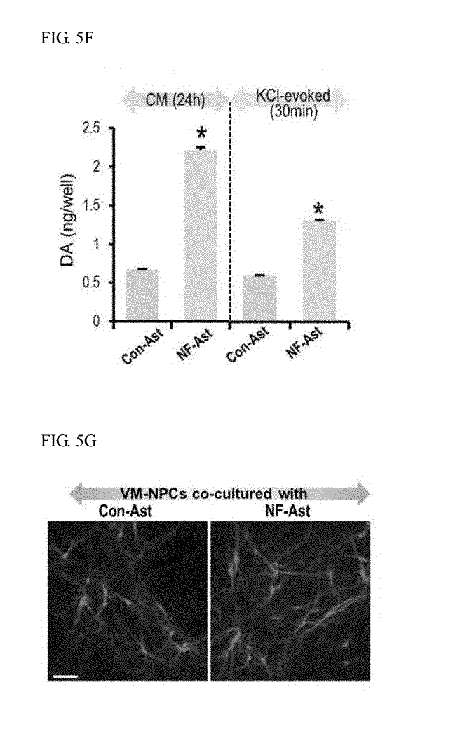

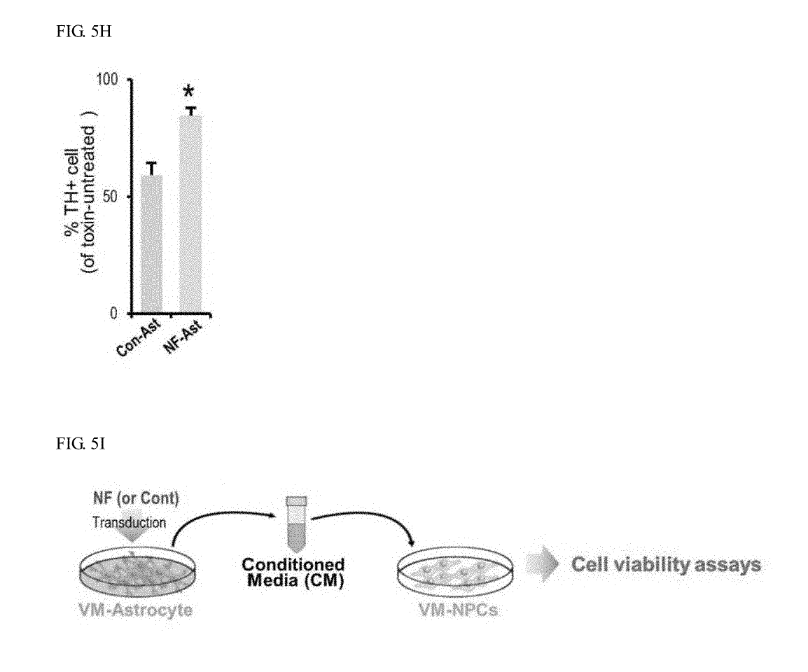

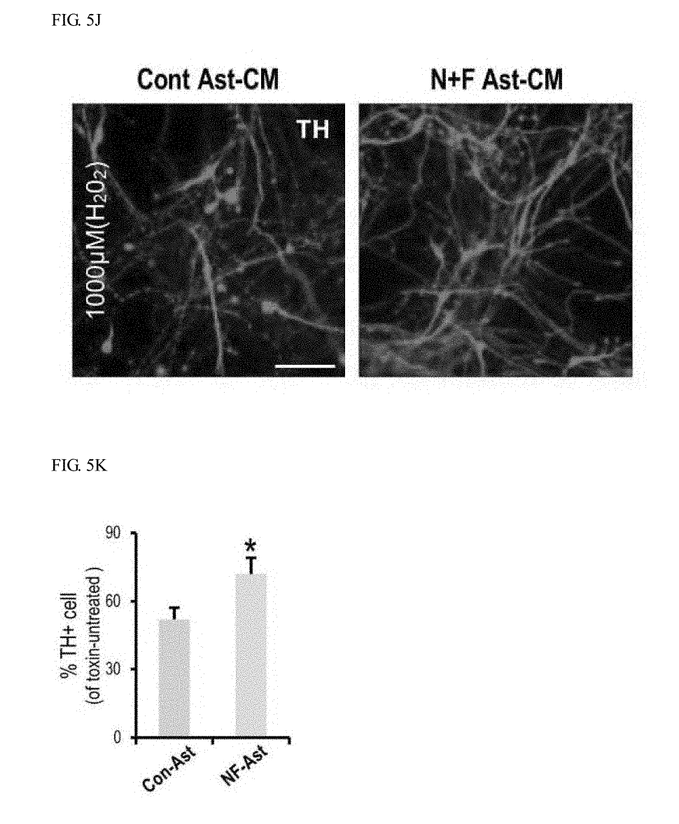

VM-astrocytes were transduced with Nurr1+Foxa2-expressing lentiviruses or mock viruses (control). The VM-NPCs harvested were co-cultured with the transduced astrocytes and the NPC behaviors associated with their therapeutic capacity upon transplantation were assessed (FIG. 5A). DA neuronal yield (FIG. 5B), morphologic (TH+ fiber lengths; FIG. 5C) and synaptic maturation (SV2+ puncta density; FIG. 5D), and the expression of midbrain-specific markers (FIG. 5E) in the DA neurons differentiated from VM-NPCs in the presence of Nurr1+Foxa2-transduced VM-astrocytes (N+F-astrocytes) were indistinguishable from cultures co-cultured with the control-astrocytes.

However, presynaptic DA neuronal functionality, as estimated by DA neurotransmitter release, was significantly greater in the cultures differentiated in the presence of N+F-astrocytes than in cultures differentiated with the control-astrocytes (FIG. 5F).

In addition, differentiated TH+ DA neurons in the presence of N+F-astrocytes were more resistant to the toxic insult induced by H₂O₂ treatment than differentiated TH+ DA neurons co-cultured with the control-astrocytes (FIG. 5G FIG. 5H). A larger number of TH+ DA neurons along with healthier neuronal shape were also observed in the cultures treated with the medium conditioned in N+F-astrocytes (N+F-CM) compared to cultures treated with control-CM (FIG. 5I-FIG. 5L), suggesting that the Nurr1+Foxa2 neuroprotective actions were mediated in a paracrine manner.

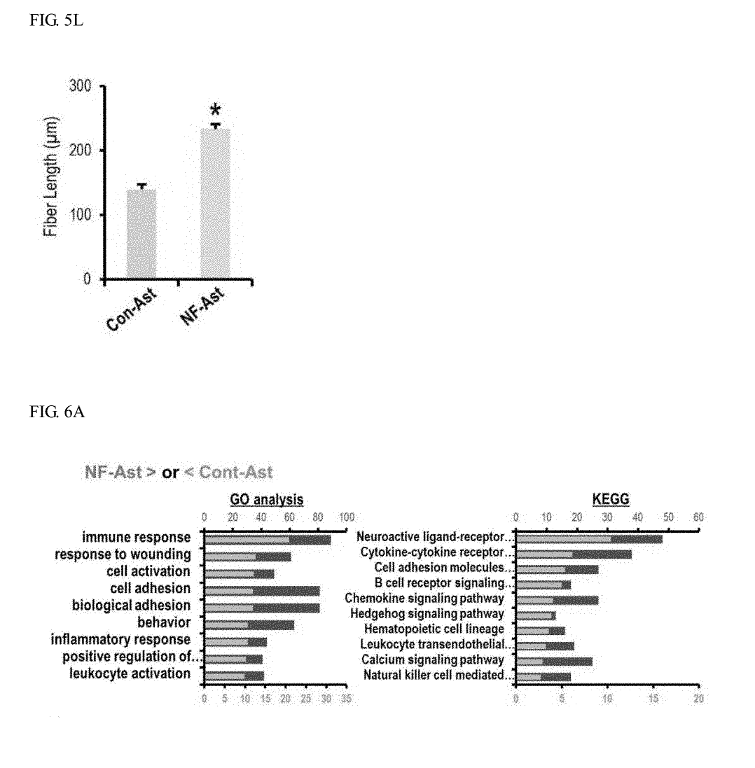

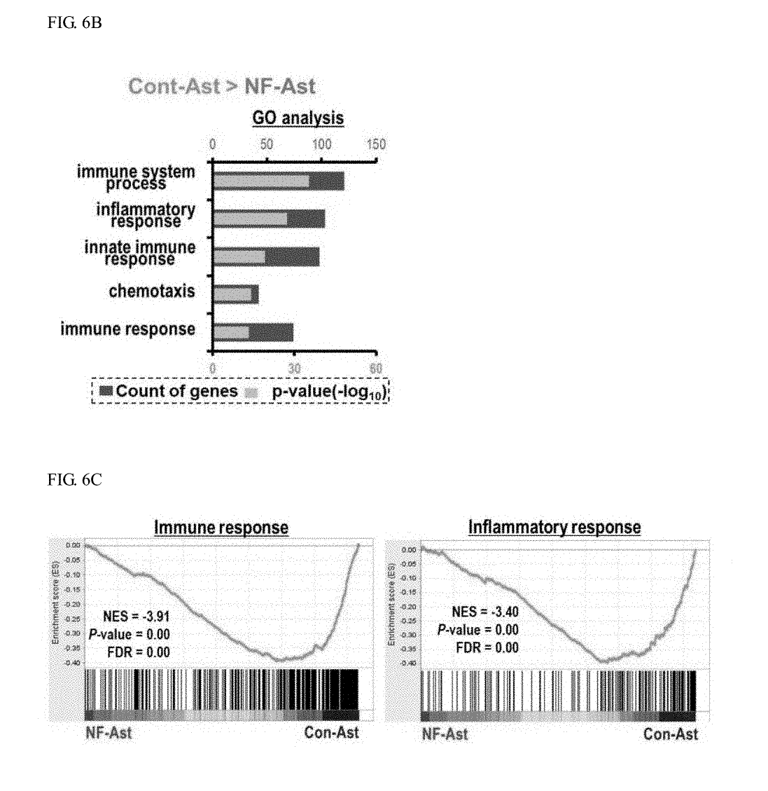

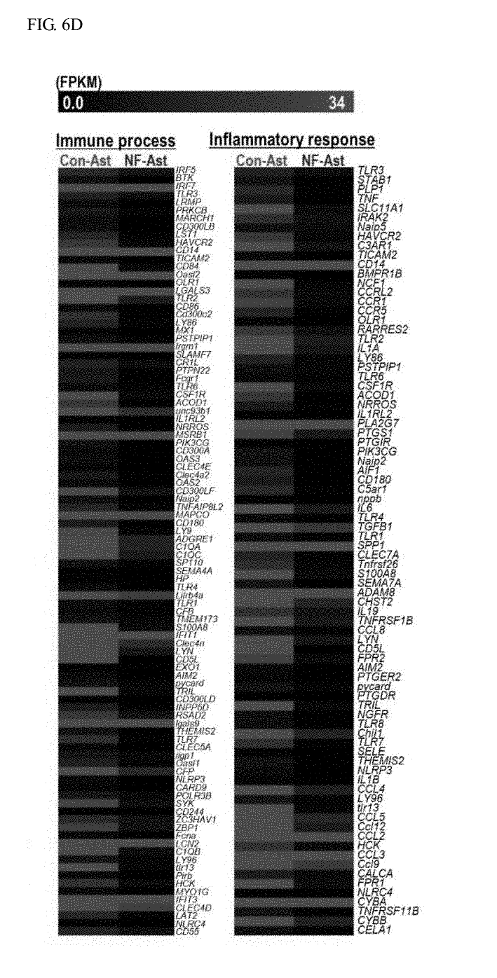

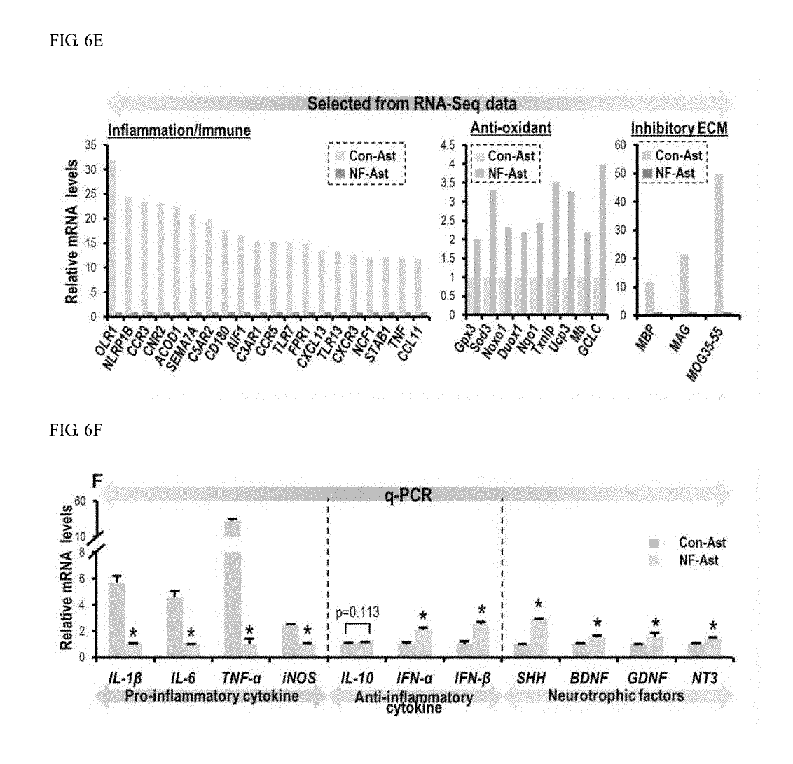

In RNA-seq analysis of the N+F-astrocytes compared to control-astrocytes, the top 10 ranked ontologies enriched by the DEGs included ‘immune/inflammation’, ‘response of wound healing’, and ‘cell adhesion’ (FIG. 6A).

In addition, when we performed gene set analysis with genes with down-regulated expression in N+F-astrocytes vs. control-astrocytes (log 2<−1), 4 out of the top 5 ontologies were related to ‘immune/inflammation’ (FIG. 6B, FIG. 6D), which was further confirmed by GSEA with curated gene sets ‘immune response’ and ‘inflammatory response’ (FIG. 6C), indicating that Nurr1+Foxa2 expression in the VM-astrocytes may mainly exert extrinsic neuroprotective roles by reducing immune/inflammation-mediated neurotoxicity.

Specifically, decreased mRNA levels of pro-inflammatory cytokines (IL-1β, IL-6, TNF-α, and iNOS) and myelin-associated proteins (MBP, MAG, and MOG) were manifested in the RNA-seq data (FIG. 6E) and those were further confirmed by qPCR analyses (FIG. 6F). In addition, increased expression of secretory neurotrophic (SHH, BDNF, GDNF, and NT-3) and anti-inflammatory (IFN-α, IFN-β) factors was also detected in Nurr1+Foxa2-astrocytes (FIG. 6F), suggesting that they also contributed to the N+F-CM-mediated neuroprotective actions.

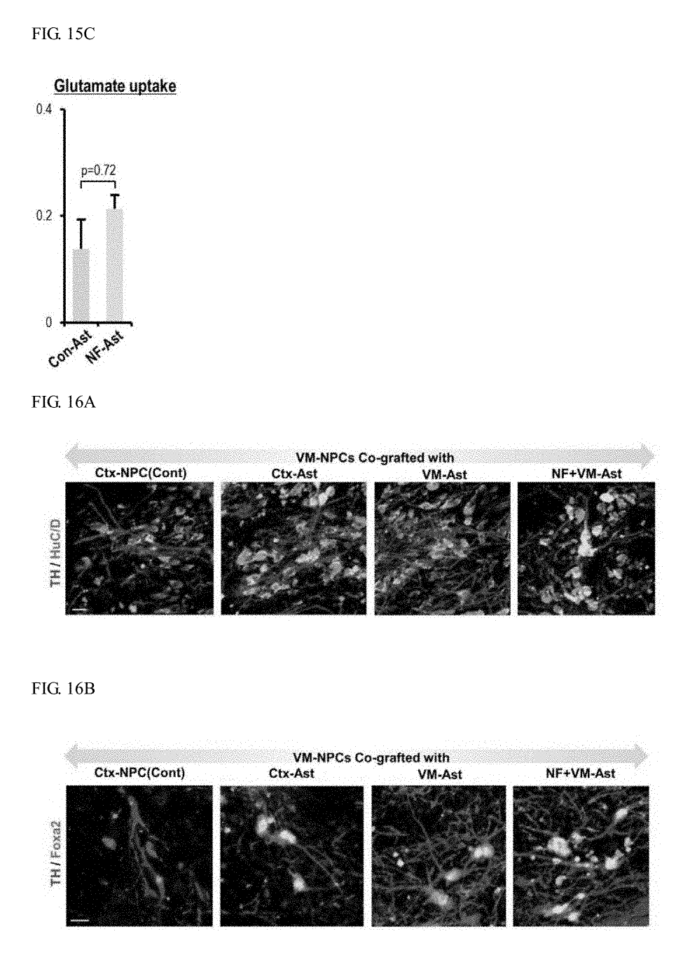

Further examination of the RNA-seq data identified multiple anti-oxidant enzymes with upregulated gene expression in N+F-astrocytes (FIG. 6E). Consistently, significantly greater intracellular glutathione levels were manifested in the N+F-VM-astrocytes compared to the control VM-astrocyte cultures (FIG. 15A).

Consistent with the upregulated secretory ROS scavenging factors SOD3 and GPX3 (FIG. 6E), ROS levels in mDA neuron-enriched cultures (differentiated from VM-NPCs) were dramatically decreased by N+F-CM treatment (FIG. 15B).

These findings collectively indicated that the enhanced ROS scavenging capacity in VM-astrocytes by Nurr1+Foxa2 also contributed to N+F-astrocyte-mediated neuroprotective actions. Glutamate uptake activity, however, was indistinguishable between control and N+F-VM-astrocytes (FIG. 15C).

Co-Grafting of Astrocytes Potentiates the Cell Therapeutic Effects of VM-NPC Transplantation via Establishing a Neurotrophic Host Brain Environment

Although our in vitro data clearly supported the neurotrophic actions of cultured astrocytes, it remains to be identified if similar astrocyte-mediated effects occur after transplantation in the host brain in vivo, where the grafted cells are inevitably exposed to hostile inflammatory/immunogenic environments and interact with endogenous cells such as glia, neuronal cells, and peripheral blood cells that may enter through the disrupted blood-brain barrier (BBB) during the cell injection process. Of note, it has been reported that glia can be reactivated into the detrimental phenotype during a delayed injury phase, raising concerns that grafted astroglia may also convert into the harmful phenotype after transplantation. Thus, our first round of transplantation experiments was performed to determine if transplantation of cultured astrocytes could establish a neurotrophic environment.

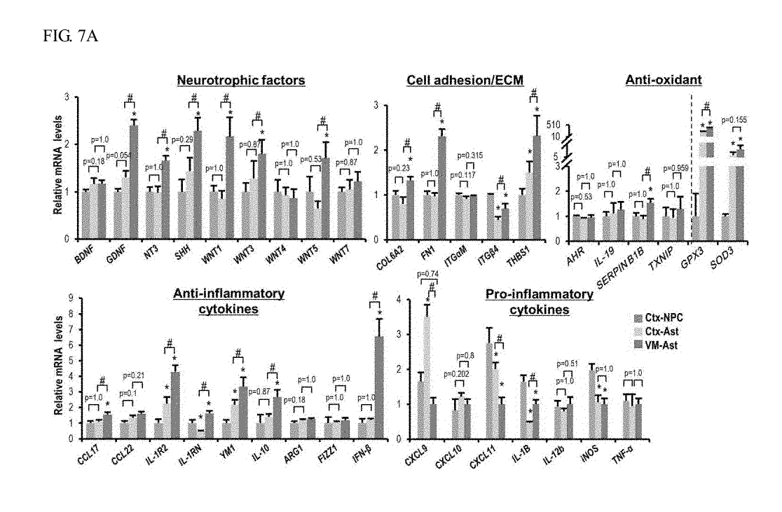

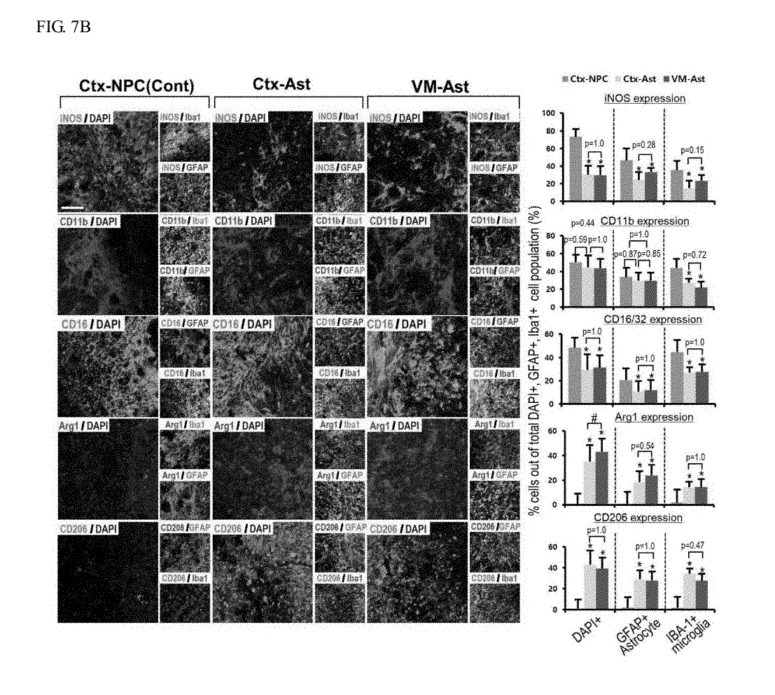

Similar to the in vitro data, the expression of neurotrophic factors (GDNF, NT3, SHH, Wnt1, 3, 5), trophic ECM proteins (COL6A2, FN1, THBS-1), and antioxidant proteins (GPX3 and SOD3) was upregulated in the striatum transplanted with VM-astrocytes, compared with the striatum grafted with control Ctx-NPCs (or Ctx-astrocytes) at 1 month post-transplantation (FIG. 7A). In addition, the expression of anti-inflammatory phenotype markers (IFN-β, CCL17, IL-1R2, IL-1RN, Ym1, and IL10) was also greater in brains grafted with VM-astrocytes.

Notably, in contrast to the in vitro findings, the expression of pro-inflammatory phenotype genes did not increase, and some genes (iNOS, IL-1β, CXCL11) were down-regulated in brains grafted with VM-astrocytes compared to brains grafted with control NPCs (FIG. 7A), suggesting that the naive VM-astrocyte property of high expression of pro-inflammatory genes in vitro was altered in the grafted in vivo brain environments probably due to the interaction with endogenous cells.

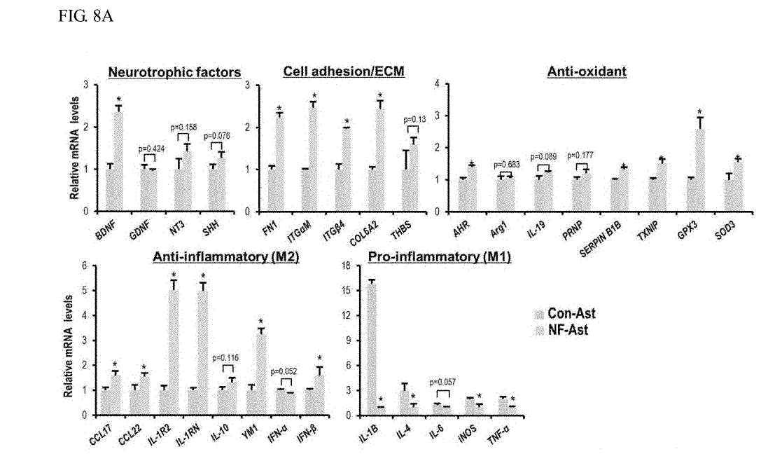

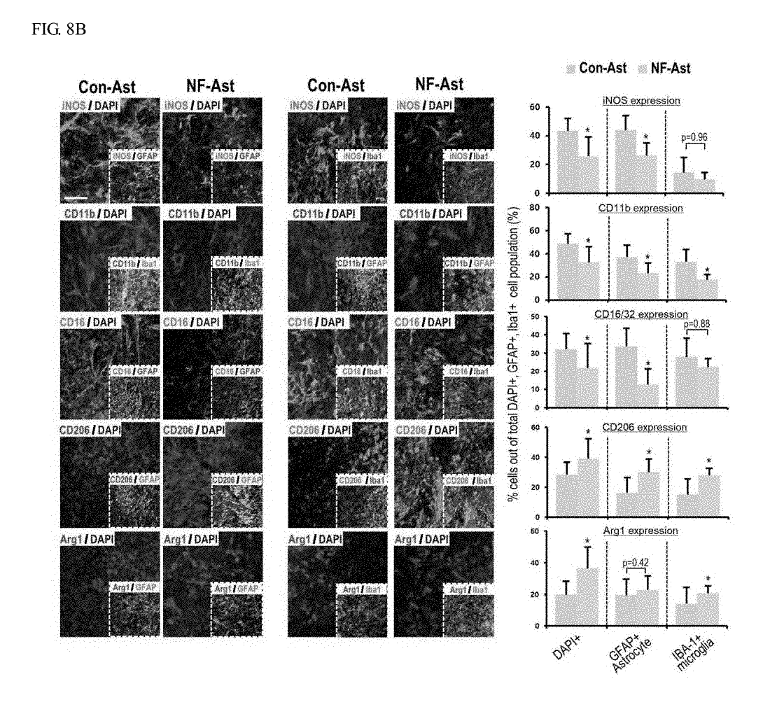

It is also possible that endogenous microglia/astrocytes were activated into the anti-inflammatory/neurotrophic phenotype due to their interaction with grafted VM-astrocytes. We further confirmed the astrocyte grafting effects by immunohistochemical analyses (FIG. 7B). Cells (astrocytes, microglia) immunoreactive for the markers specific for anti-inflammatory/neurotrophic glia (Arg1, CD206) were significantly greater in the graft-host interfaces of the brain transplanted with cultured astrocytes, compared to those grafted with Ctx-NPCs, along with reduction of the cells expressing pro-inflammatory/cytotoxic factors (iNOS, CD11b, CD16). An upregulated expression pattern of neurotrophic, antioxidant, and ECM genes was further observed in the brains grafted with N+F-transduced VM-astrocytes compared to brains transplanted with control VM-astrocytes (FIG. 8A).

The phenotype transition by N+F-VM-astrocyte transplantation was further clarified in immunohistochemical analyses exhibiting downregulated expression of pro-inflammatory/cytotoxic phenotype markers and upregulated expression of anti-inflammatory/neurotrophic phenotype markers in the brains transplanted with N+F-VM-astrocytes compared to brains grafted with control VM-astrocytes (FIG. 8B).

Taken together, these findings indicate that transplantation of cultured VM-astrocytes could establish a neurotrophic brain environment, and that Nurr1+Foxa2 expression in the donor astrocytes potentiates the astrocyte transplantation effect.

We ultimately assessed if co-grafting astrocytes could improve cell therapeutic outcomes in PD. VM-NPCs mixed with cultured VM- or Ctx-astrocytes (or Ctx-NPCs as control) were intrastriatally transplanted into a hemi-parkinsonian rat model and PD-related behaviors were assessed for 6 months after transplantation.

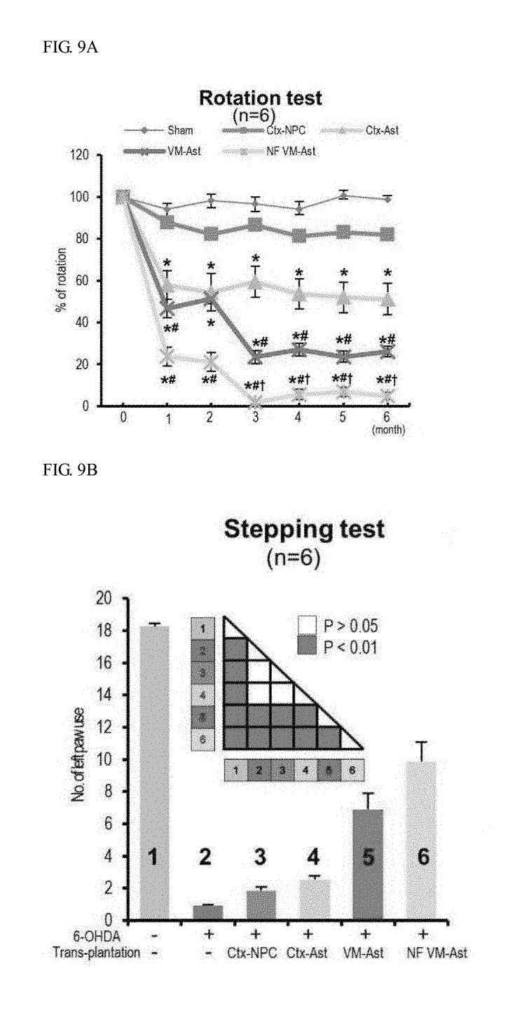

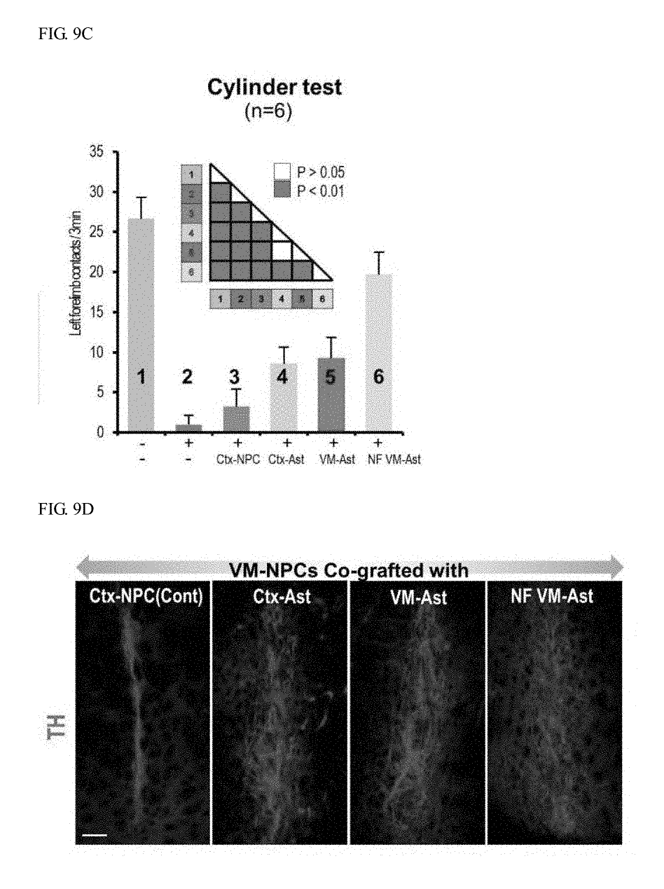

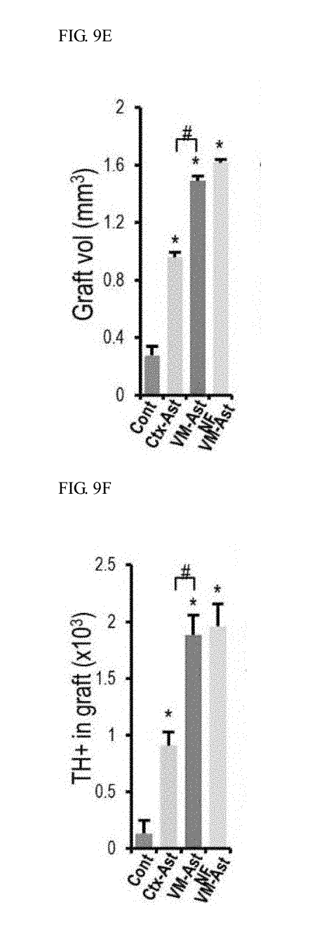

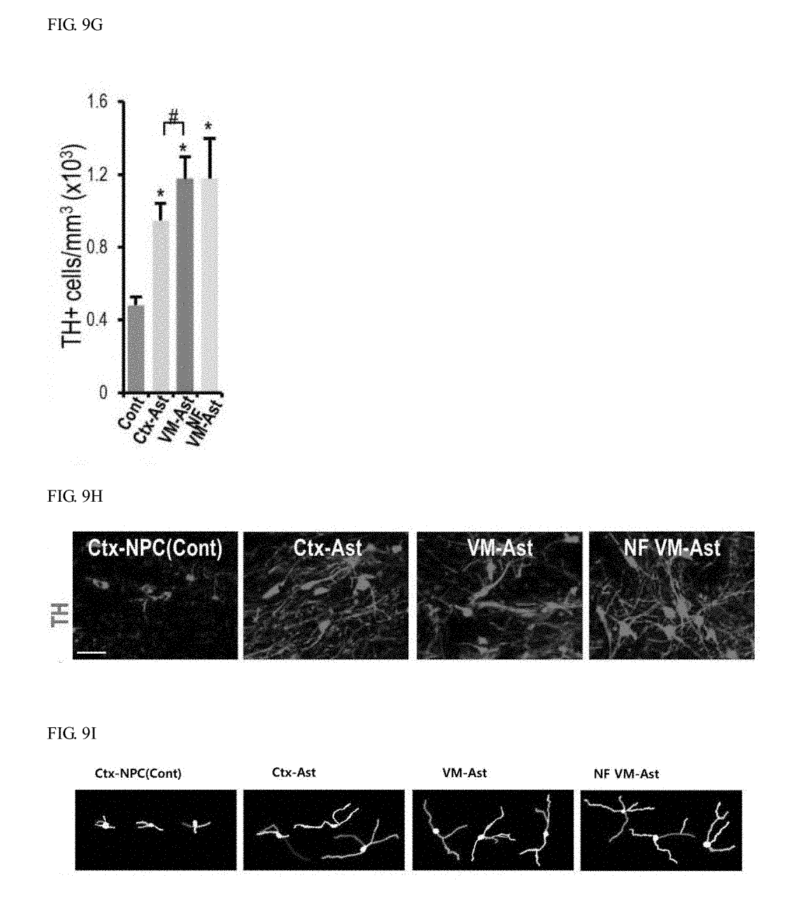

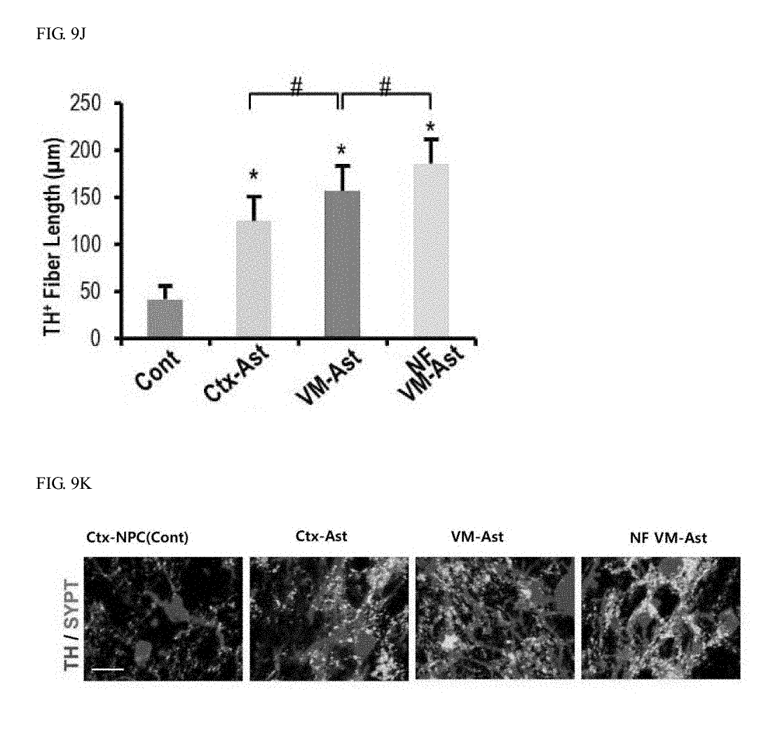

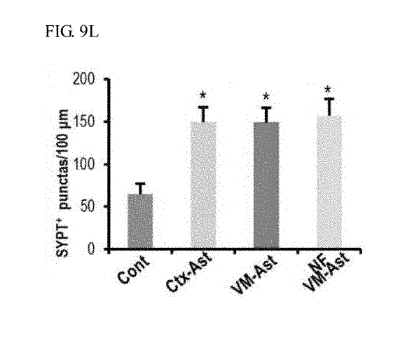

An amphetamine-induced rotation test revealed only a minor reduction in rotation scores compared to pre-transplantation values in PD rats co-grafted with VM-NPCs+Ctx-NPCs (control), but greater behavioral recovery was achieved by co-grafting with astrocytes (FIG. 9A).

Especially, co-grafting of N+F-VM-astrocytes along with VM-NPCs resulted in an almost complete recovery in the rotation scores (>95% reduction in rotation score from 3 months post-transplantation, n=6).

We also assessed PD behaviors using non-pharmacological assays at 6 months post-transplantation.

Similar patterns of behavioral recovery among the animal groups were observed in the step adjustment (FIG. 9B) and cylinder tests (FIG. 9C).

Consistent with the data from the in vitro co-culture and CM experiments as well as the behavioral assays, histologic analyses performed 6 months post-transplantation exhibited formation of a much larger TH+ cell graft in rats co-transplanted with astrocytes than with control NPCs (FIG. 9D, FIG. 9E). All indices for DA neuron engraftment (FIG. 9E-FIG. 9G) were significantly greater in animals co-grafted with VM-astrocytes compared to Ctx-astrocytes, but no significant difference in the indices was detected between the animal groups co-grafted with VM-astrocytes vs. N+F-VM-astrocytes. TH+ cells in the striatum co-grafted with astrocytes exhibited healthier and more mature neuronal maturity than those in the control grafts (FIG. 9H, FIG. 9I), which was confirmed by the estimation of TH+ fiber length (FIG. 9J) and synapsin+ puncta density (FIG. 9K, FIG. 9L). Only a slight but significant increase in the TH+ fiber lengths was detected in the N+F-VM-astrocyte-co-grafted groups, compared to those co-grafted with the control-VM-astrocytes (FIG. 9J).

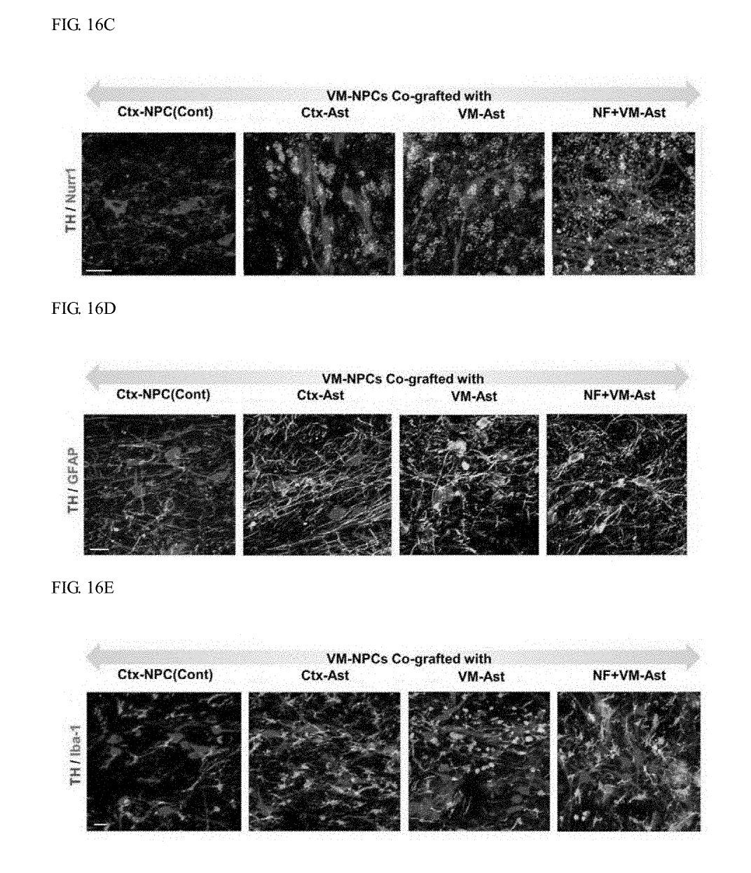

Surprisingly, the midbrain-specific markers Nurr1 and Foxa2, expression of which are easily lost in donor cells after transplantation, were faithfully co-localized in TH+ DA neurons in the grafts generated by astrocyte co-transplantation, especially by co-grafting with N+F-VM-astrocytes, even 6 months after transplantation (FIG. 16A-C). TH+ DA neuronal cells in the grafts were surrounded by GFAP+ astrocytes (probably both transplanted and endogenous) (FIG. 16D) and Iba1+ microglia (endogenous) (FIG. 16E).

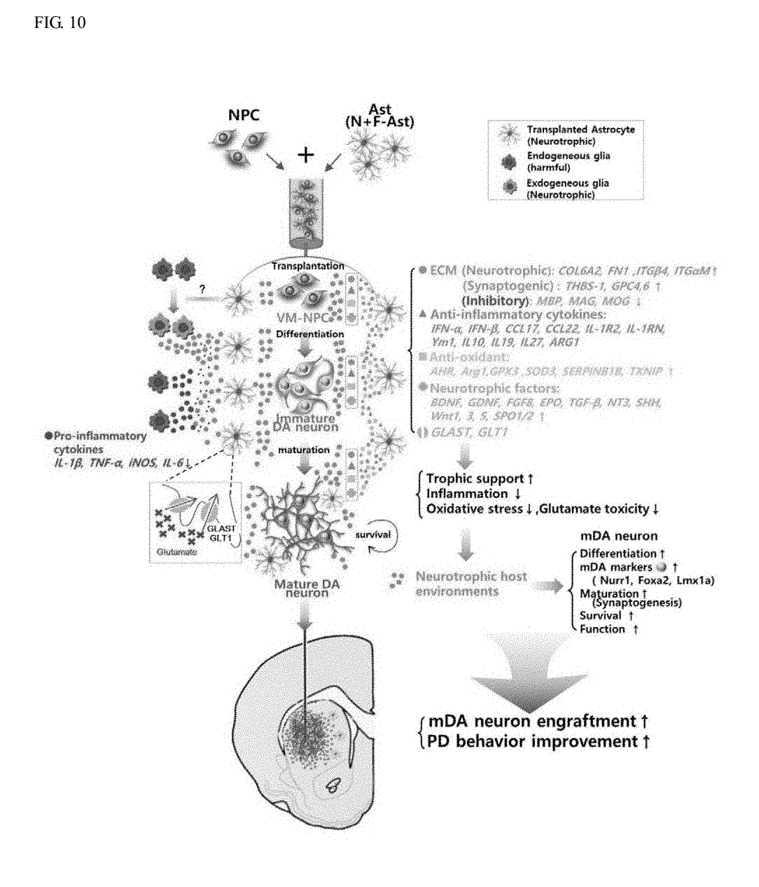

The Iba1+ microglia were ramified with a resting or neuroprotective morphology, indicating that the neighboring and covering of the grafted DA neurons was protective. These findings collectively indicated that co-transplantation of VM-NPCs with astrocytes, especially with N+F-VM-astrocytes, ensured a long-term engraftment of mature authentic mDA neurons via astrocytic actions to improve host brain environments (schematized summary is shown in FIG. 10).

The Transmission of α-Synucleinopathy from the Host Brain of a Parkinsonian Patient to the Grafted Cells Through Cell Transplantation Had Become a Huge Issue in Cell Transplantation Therapy for PD.

Normally, α-synuclein is a protein expressed in neuronal cells and playing a normal role of transmitting nerve signals, etc., but under circumstances in which an abnormal pathologic aggregate (called a Lewy body) is formed due to an increase in amounts of α-synuclein or under a pathologic environment, it becomes the cause of a neurodegenerative disorder such as PD. Therefore, the clearance of α-synucleinopathy due to the α-synuclein aggregate is a solution to the prevention and treatment of PD.

However, the α-synuclein aggregation has a characteristic of cell-to-cell transmission like a prion, and it has been observed in the brain of a patient with PD after death that α-synuclein had transmitted from a host to a graft.

The inventors found from RNA-Seq analysis that the expression of genes related to the inhibition and clearance of α-synuclein aggregation (INF-alpha and INF-beta) was increased in astrocytes, particularly, VM-astrocytes (VM-Ast) [FIG. 4F].

In addition, it was found that genes related to phagocytosis and autophagy are highly expressed in VM-astrocytes (FIG. 20).

It has been known that inflammation promotes pathologic protein aggregation, and that anti-inflammatory, antioxidative and neurotrophic materials inhibit the formation of a pathologic protein aggregate, and the inventors discovered that neurotrophic, antioxidative and anti-inflammatory actions are highly increased in the proximity of a site engrafted with VM-astrocytes (FIG. 7A).

Based on the result of the above-mentioned research, it was investigated whether the occurrence of α-synucleinopathy is prevented in grafted cells by the inhibition and clearance of α-synuclein aggregation and inhibition of intercellular transmission when ventral midbrain neural stem cells and astrocytes are co-grafted into a mouse PD model experiencing α-synucleinopathy.

1) Confirmation of Effect of Co-Culture of Astrocytes with α-Synuclein-Overexpressing DA Neurons on Reduction in α-Synuclein Aggregation

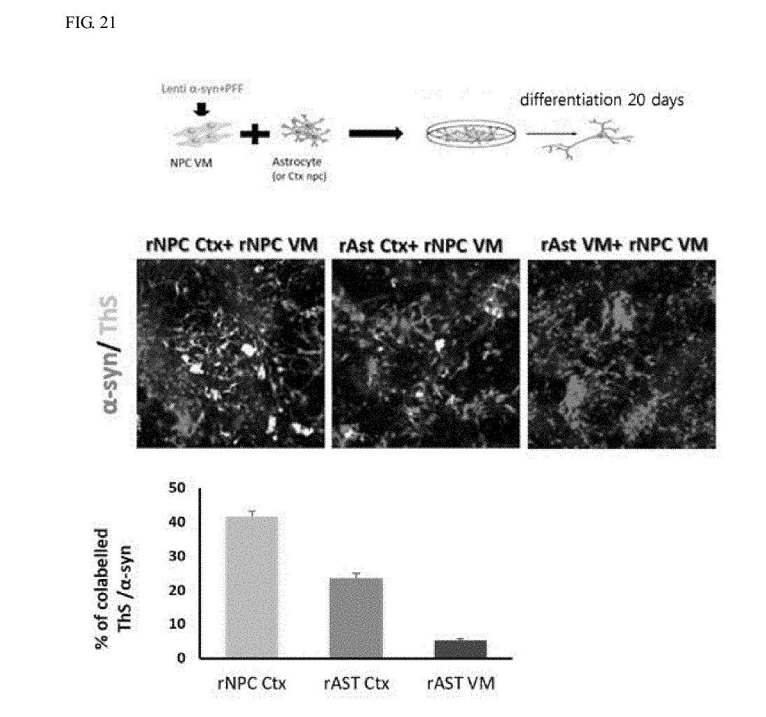

Compared with the control, when DA neurons were co-cultured with astrocytes, particularly, VM-astrocytes, α-synuclein aggregation was reduced, as confirmed by α-synuclein/thioflavin S immunocytochemistry (thioflavin S: staining for detection of pathologic protein aggregates) and western blotting (FIGS. 21 and 22).

2) Inhibitory Effect on Intercellular α-Synuclein Transmission and Aggregation Due to Factor Secreted from Astrocytes

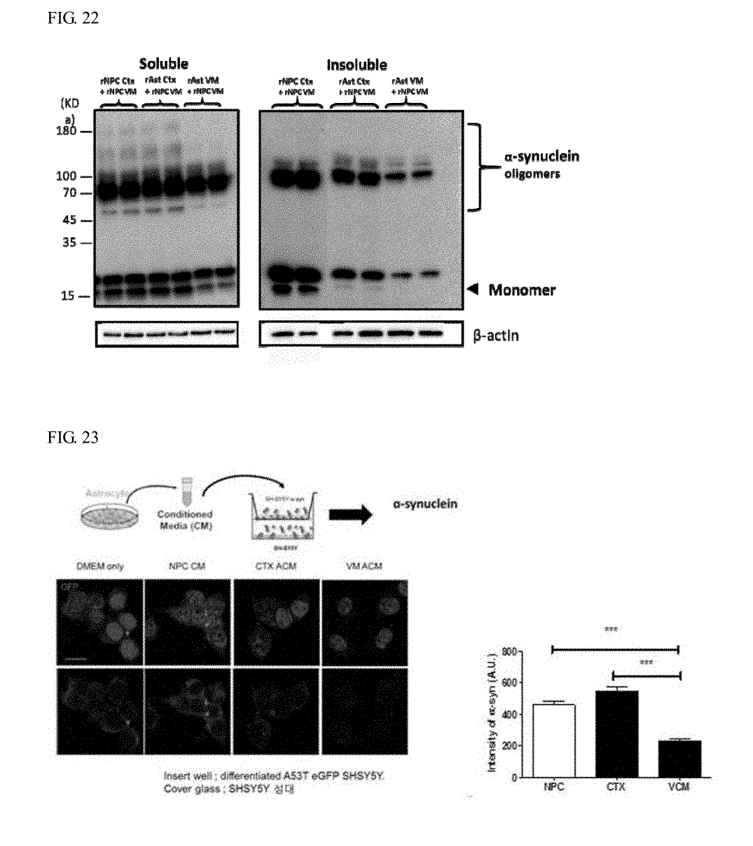

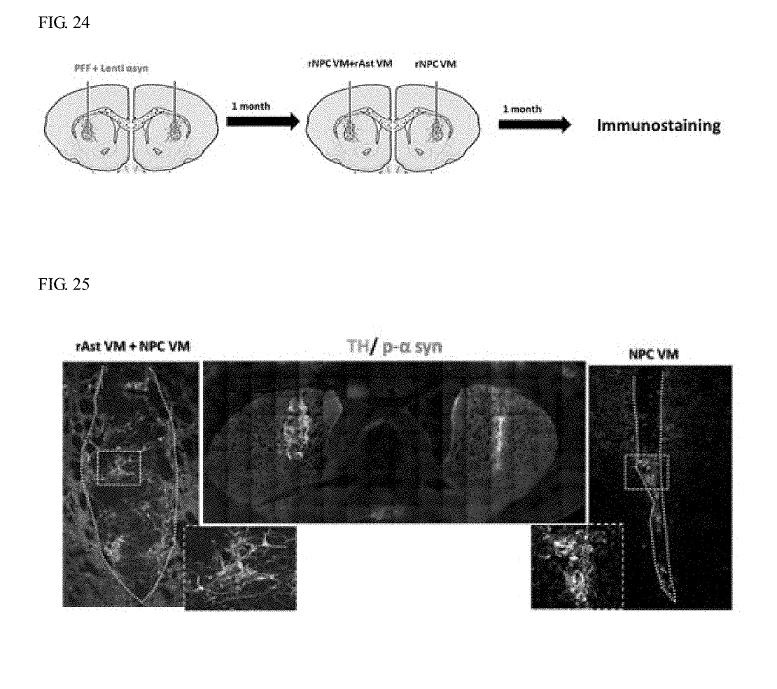

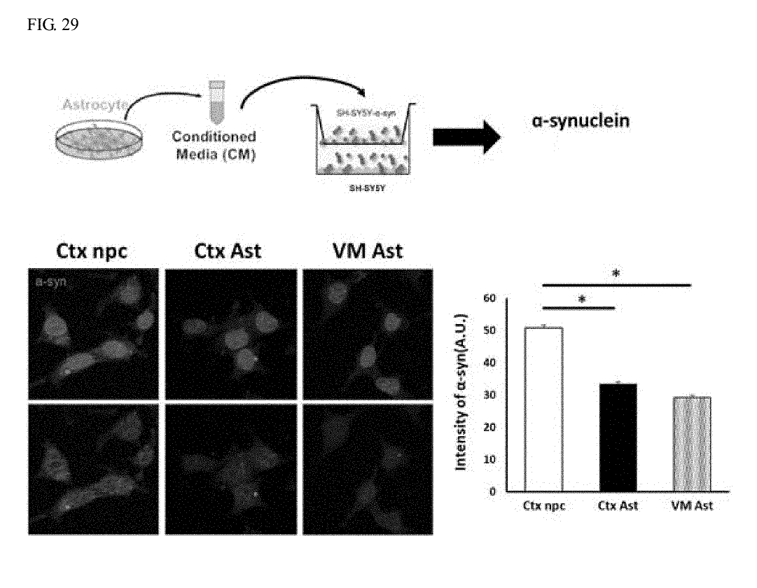

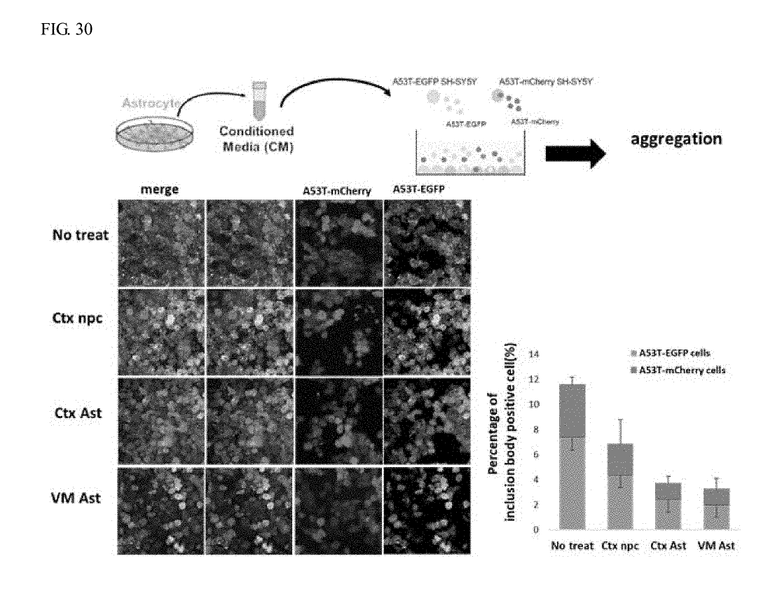

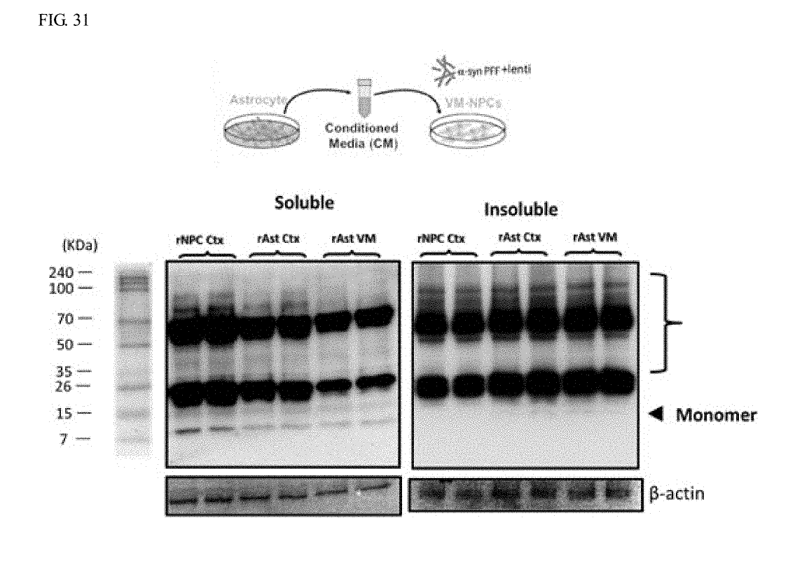

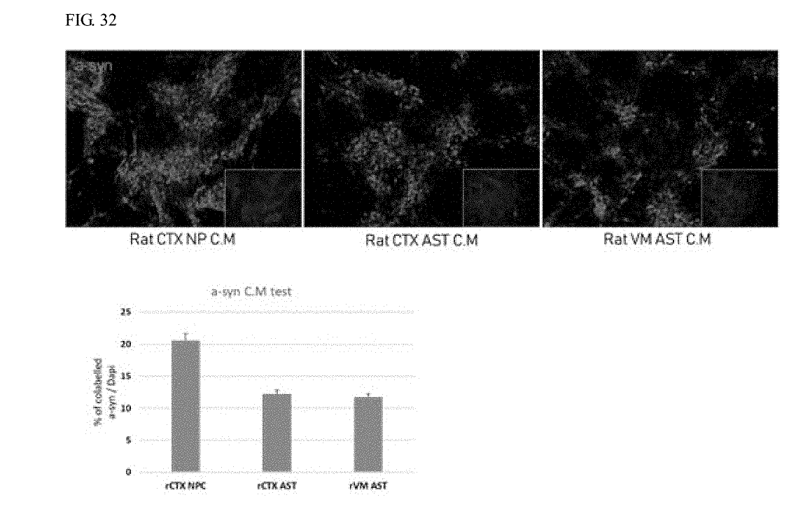

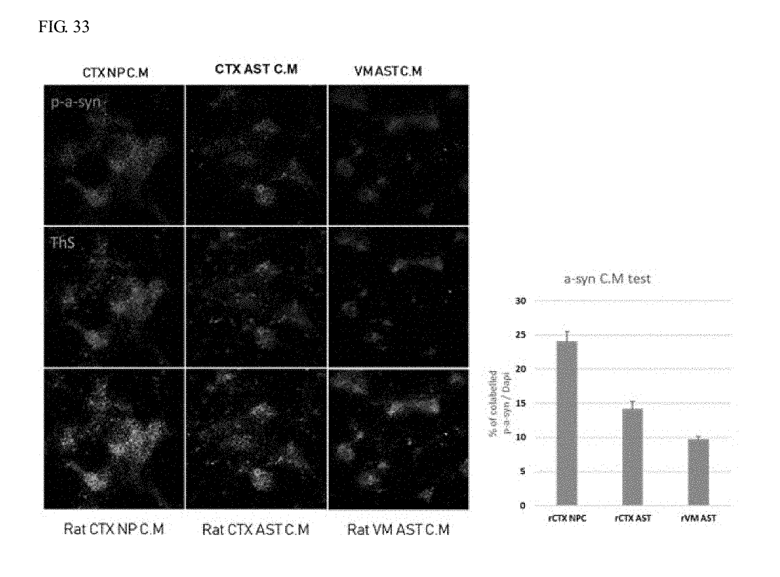

It was confirmed that a conditioned medium of astrocytes, particularly, VM-astrocytes, inhibited α-synuclein transmission to neuronal cells more effectively than the control and cortex astrocytes (FIG. 23), and even when SH-SY5Y cells were used to overexpress α-syn-GFP, α-syn-mCherry and α-synuclein, and co-cultured, aggregation was also inhibited, as confirmed by measuring expression levels of GFP and mCherry (FIGS. 29 to 33). Therefore, it was confirmed that factors released from the astrocytes, as well as the astrocytes themselves, play a major role in α-synuclein transmission and clearance from the transmitted cells.

3) Effect of Factor Secreted from Astrocytes on Decomposition of α-Synuclein Aggregate

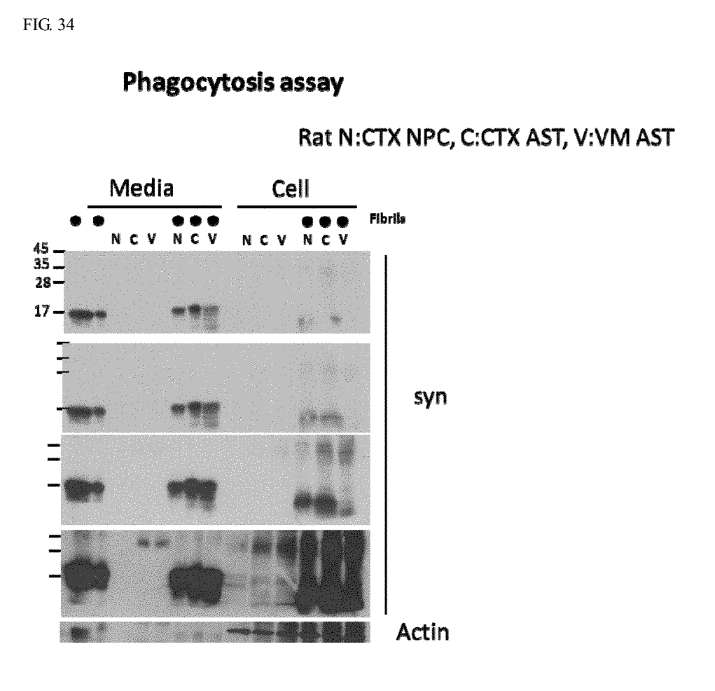

By treating ventral midbrain astrocytes with PFF and then monitoring the remaining PFF, it was confirmed that α-synuclein aggregates were decomposed (FIG. 34), and that α-synuclein aggregates were decomposed by a specific material secreted from the astrocytes.

4) Effect of Co-Grafting of VM-Ast into α-Synucleinopathy-Induced Mouse PD Model on Inhibition of α-Synuclein Aggregation in Grafted Cells

Consequently, it was observed that a graft obtained by co-grafting of ventral midbrain neural progenitor cells (VM-NPC) and ventral midbrain-derived astrocytes (VM-Ast) had more DA neurons and a larger size than the graft in the control transplanted only with ventral midbrain neural progenitor cells (VM-NPC), and it was confirmed that whereas p-αsyn (aggregation form of α-syn) neither permeated into the graft obtained by co-grafting nor was it observed in the proximity of the graft, p-αsyn was observed in the control transplanted only with ventral midbrain neural progenitor cells (VM-NPC) (FIG. 25).

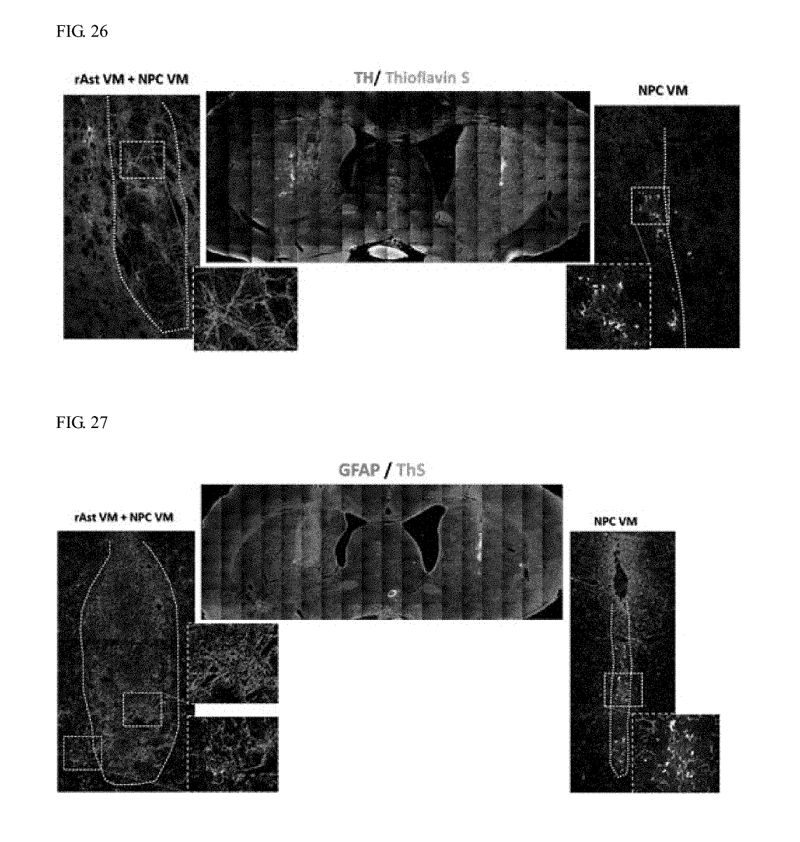

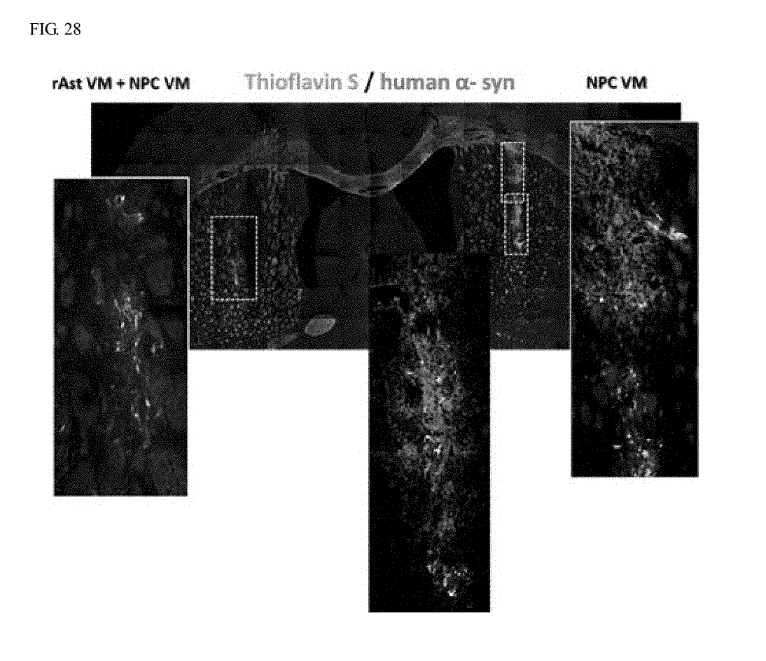

In addition, it was also observed that thioflavin S, which enables the observation of a protein aggregation form, is present in the proximity without being able to permeate into the graft in the case of the astrocyte group like p-αsyn, whereas in the control, thioflavin S permeated into the graft such that the DA neurons died (FIG. 26).

To see that such an effect is caused by astrocytes, staining with GFAP, an astrocyte marker, and thioflavin S showed that many astrocytes are present in the graft formed with ventral midbrain neural progenitor cells (VM-NPC) and ventral midbrain-derived astrocytes (VM-Ast), and that whereas thioflavin S was not observed in such astrocytes, in the control transplanted only with ventral midbrain neural progenitor cells (VM-NPC), GFAP was hardly observed and thioflavin S was expressed in many cells of the graft (FIG. 27).

5) Effect of Co-Grafting with Human VM-Ast on Inhibition of Inflammation of Grafted Cells (Graft) in Inflammation-Induced Rat PD Model

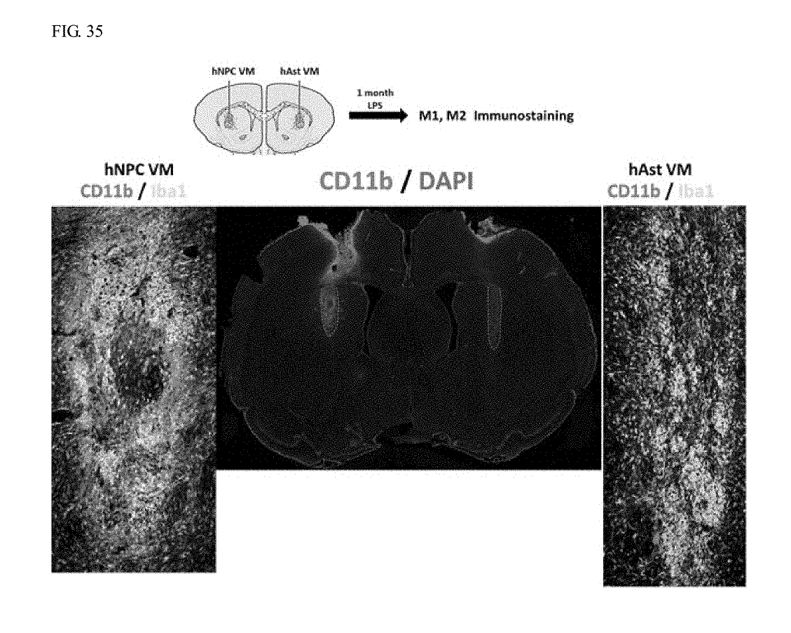

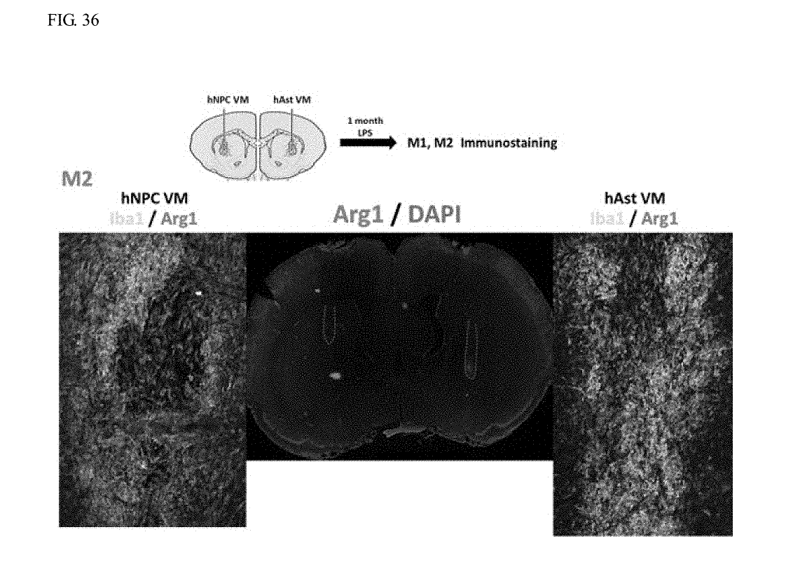

To confirm possibility of anti-inflammatory effect of astrocytes, inhibition of inflammation of grafted cells (graft) after grafting with astrocytes was monitored with transplantation of human ventral midbrain neural progenitor cells (VM-NPC) to the left side of the rat striatum and human ventral midbrain-derived astrocytes (VM-Ast) to the right side thereof. It was confirmed that in human ventral midbrain-derived astrocytes (VM-Ast), inflammatory responses [CD11b (M1 type staining)/Iba1 (microglia staining)] are reduced compared with human ventral midbrain neural progenitor cells (VM-NPC) (FIG. 35), and anti-inflammatory responses [Arg1(M2 type staining)/Iba1(microglia staining)] are increased (FIG. 36).

6) Confirmation of Effect of Co-Grafting of VM Ast on Survival and Maintenance of Stable DM Neurons in α-Synucleinopathy-Induced Mouse PD Model

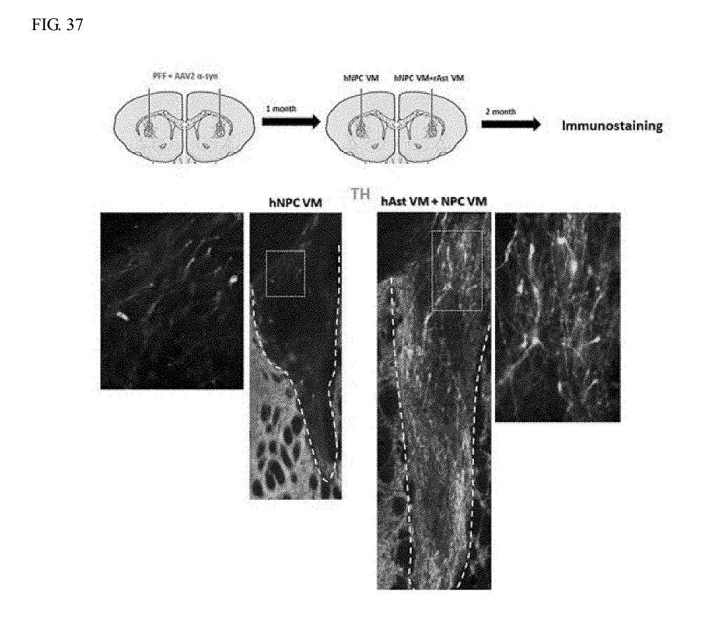

It was confirmed that, in graft co-grafted with human VM-NPCs and VM-Asts, more DM neurons survived and well maintained than the control single-grafted with VM-NPCs, and a larger graft is formed (FIG. 37).

[Discussion]

It has long been suggested that after transplantation, the host brain becomes hostile to grafted cells, and this detrimental host brain environment is mainly responsible for unsatisfactory cell transplantation therapeutic results with poor neuronal engraftment. Nevertheless, targeting the host brain to improve cell therapeutic effects has only been addressed in a few studies.

Furthermore, none of these studies used an approach to fundamentally correct inflammatory cytotoxic host brain environments. Based on the physiologic neurotrophic functions of astrocytes, utilization of this cell type is a potential strategy to modify pathologic brain environments.

However, few studies have examined developing an astrocyte-based therapy for neurologic disorders, mainly because of the potential reactivation of this cell type into a detrimental/neurotoxic phenotype in diseased brains.

However, cumulative studies have demonstrated that cultured astrocytes generally exhibit immature properties as verified in FIG. 11D-FIG. 11H of our study, and that transplanted immature astrocytes do not undergo harmful reactivation after CNS injury, but rather support neurite outgrowth and reduce glial scar formation in the injured CNS.

Based on these findings, we attempted co-grafting with astrocytes to attain an improvement in therapeutic efficacy of cell transplantation for PD.

As shown in FIG. 11A, astrocyte cultures are frequently contaminated more and less with microglia. Microglia induce pro-inflammatory/cytotoxic reactivation of astrocytes. In addition, it has been reported that neurotrophic functions of astrocytes also require minor populations of microglia (<1%).

To determine if microglia contamination affects neurotrophic functions of astrocytes, we generated microglia-free astrocyte cultures by modifying the culture protocol (including one round of mild trypsin treatment step, see Materials & Methods).

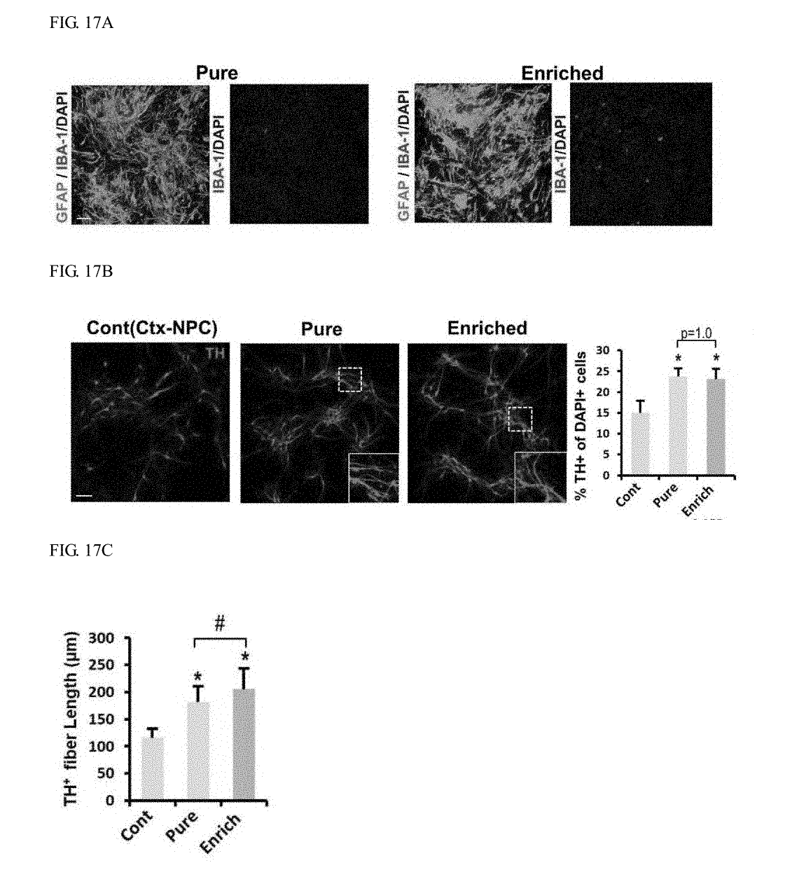

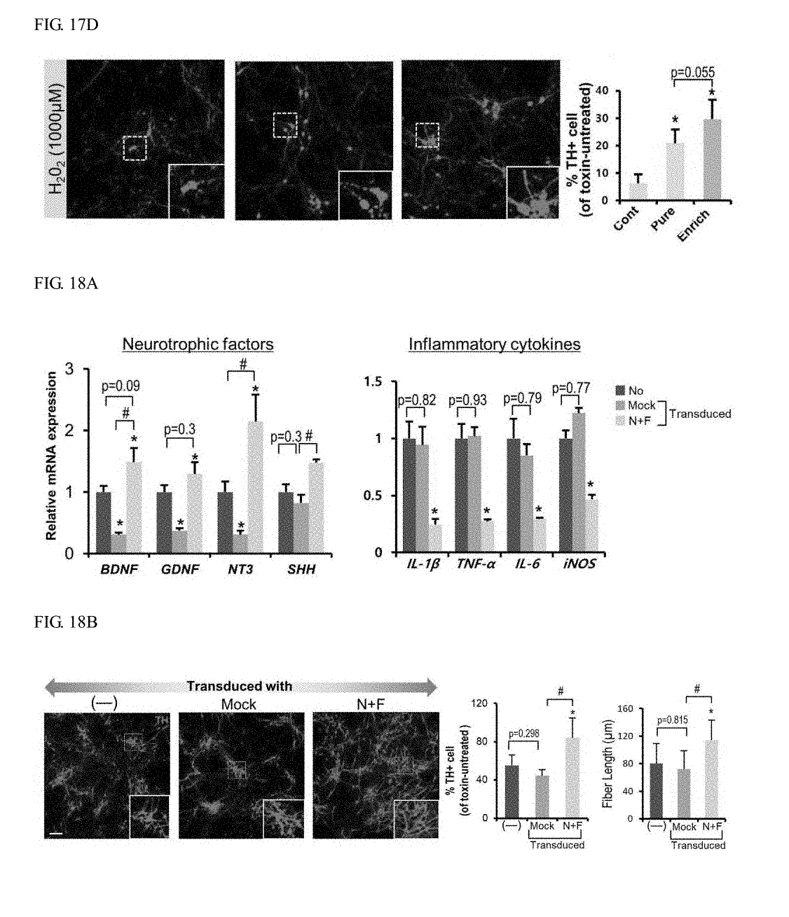

mDA neuronal differentiation, morphologic maturation, and toxin resistance promoted by CM derived from pure astrocyte cultures were comparable (or slightly lower) with those promoted by the CM from the astrocyte cultures containing the minor microglia population (FIG. 17A-FIG. 17D), indicating that astrocytes in culture can exert the observed trophic actions in the absence of microglia, and minor microglia contamination (in this study, <0.5%) also does not affect the naive neurotrophic actions of astrocytes.

Whether further, higher levels of microglia contamination are detrimental or beneficial remains to be identified. Like neurons, astrocytes are also thought to have regional identities and play region-specific roles. Thus, astrocytes cultured from the VM neurogenic niche were expected to be dopaminotrophic. Previous studies have consistently demonstrated that astrocytes derived from the VM facilitate DA neuron differentiation and survival via secretion of GDNF, FGF2, and Wnts.

In this study, we carried out further systematic and comparative analyses both in vitro and in vivo after transplantation to test the dopaminotrophic functions of cultured VM-astrocytes, in comparison with astrocytes cultured from the non-dopaminergic region of the cortex, and the effects of priming the VM-astrocytes with Nurr1+Foxa2, which has potentiated the neurotrophic functions of glia.

In the niche established by the VM-astrocytes, grafted NPCs efficiently differentiated into morphologically, synaptically, and functionally mature mDA neurons and the differentiated mDA neuronal cells survived for long periods after transplantation while maintaining expression of midbrain-specific markers.

The expression of midbrain-specific factors such as Nurr1 and Foxa2 is a critical indicator for successful mDA neuron engraftment, as the expression of these genes is required for mDA neuron survival, function, and phenotype maintenance.

Considering that the expression of midbrain factors is easily lost in stressful conditions, sustained expression of midbrain markers in the presence of astrocytes is likely to be attained by the observed astrocyte actions which change the hostile inflammatory host brain milieu into a neurotrophic environment.

Mechanisms underlying the astrocyte-mediated dopaminotrophic niche included secretion of the reported neurotrophic factors, as well as other cytokines such as SHH and FGF8, which had much greater levels of expression in cultured VM-astrocytes than Ctx-astrocytes. It is likely that the expression of SHH and FGF8 is regulated by the midbrain-specific transcription factors Foxa2 and Lmx1a, respectively, expressions of which were maintained at high levels in cultured VM-astrocytes, in a positive regulatory loop in VM-astrocytes.

In addition, VM-astrocytes exhibited increased synthesis of various cell-cell contact and ECM molecules including synaptogenic ECMs thrombospondins and glypicans, and enhanced glutamate clearance activity, both of which were superior to cortex-derived astrocytes. Engineering of the midbrain-specific factors Nurr1+Foxa2 in the VM-astrocytes further improved their neuroprotective action mainly by reducing inflammation as well as by enhancing ROS scavenging activity. Based on the observed Nurr1+Foxa2 functions, priming these factor expressions in astrocytes is highly suggested prior to co-grafting. However, currently available methods for exogene expression including lentiviral transduction used in this study, are more and less toxic to cells, and thus may reduce naive cellular functions.

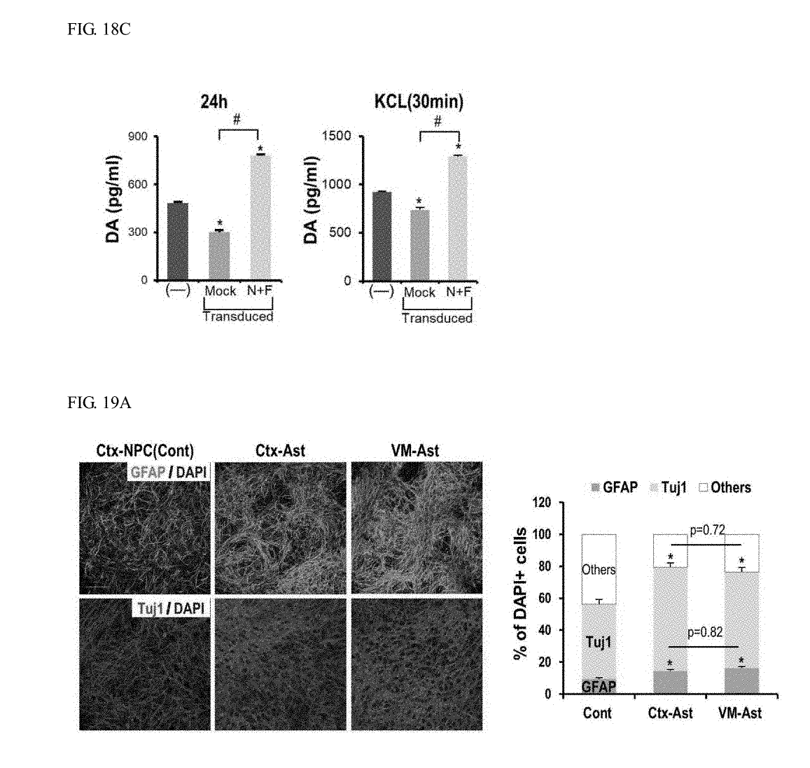

Indeed, a side-by-side comparative analysis has shown that DA release and resistance of DA neurons against H₂O₂ toxin were significantly lower in the mDA neuron cultures treated with the CM prepared in mock-transduced VM-astrocytes than those treated with CM from non-transduced VM-astrocytes, along with decreased neurotrophic factor expressions in the viral transduced astrocytes (FIG. 18).

Forced Nurr1+Foxa2 expression effects were dramatic and sufficiently overcame the viral transduction-mediated reduction of astrocyte functions and showed significantly greater neurotrophic actions than the non-transduced control in all the assays. Nevertheless, development of an exogene expression system with minimal side effects is required to maximize the Nurr1+Foxa2-mediated trophic actions.

The effects of astrocyte co-grafting in this study were dramatic, with almost complete behavioral restoration and extensive DA neuron engraftments co-grafted for at least 6 months after transplantation in PD rats.

3,762 and 3,916 TH+ DA neuronal cells per animal were detected in PD rats co-grafted with VM-astrocytes and N+F-VM-astrocytes, respectively (without any modifications to the donor cells) 6 months after transplantation, strongly indicating the necessity of a host brain modification strategy, especially for long-term donor cell survival and therapeutic efficacy.

In conclusion, we propose astrocyte co-grafting as a future option in cell therapeutic approaches for PD.

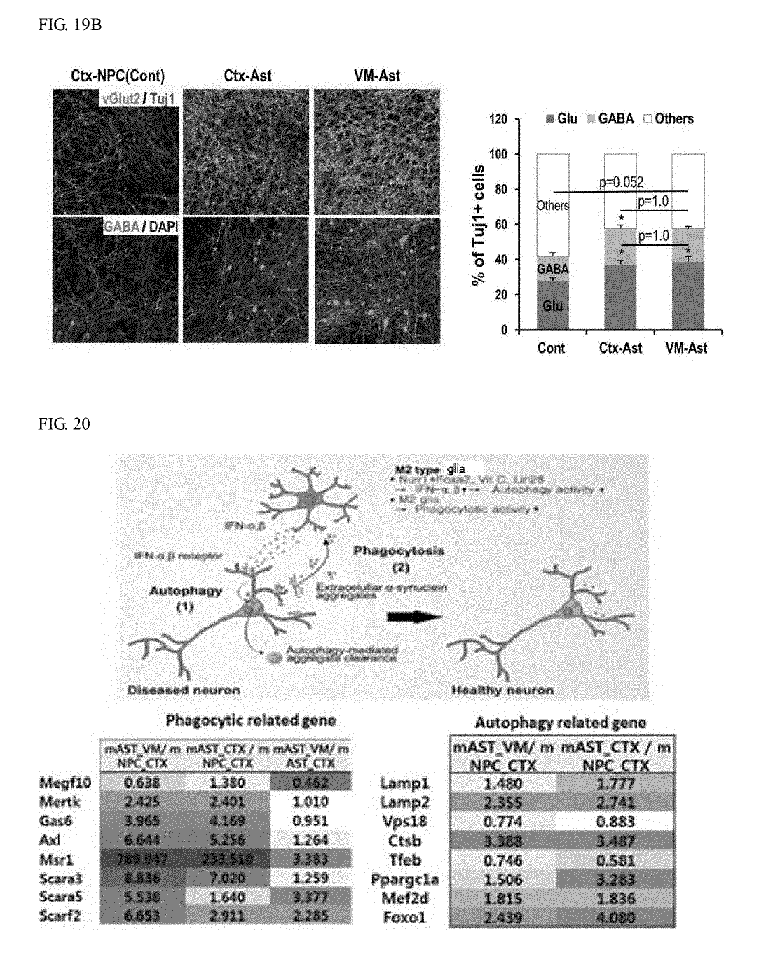

In addition, grafting astrocytes alone could exert therapeutic efficacy by improving brain environments, in which remaining endogenous mDA neurons in the SN extend axonal outgrowths to release DA into the striatum, and striatal GABAergic interneurons are rescued. Astrocytes exert pan-neuronal trophic actions, in which total neuronal yields including those of glutamatergic and GABAergic neurons are also promoted by factors released from cultured astrocytes (FIG. 19A-FIG. 19B).

Thus, the astrocyte co-grafting strategy could be utilized to treat other CNS disorders.

In addition, according to the in vitro and in vivo experiments of co-grafting of ventral midbrain astrocytes and dopamine neural progenitor cells, it was confirmed that a decrease in the transmission, aggregation and clearance of α-synuclein is affected by VM-astrocytes.

That is, in the case of α-synucleinopathy, through co-grafting of ventral midbrain-type dopamine neural progenitor cells and astrocytes, α-synuclein aggregation and clearance, and transmission to grafted DA neurons were prevented, allowing DA neurons to survive and be maintaned. Therefore, the co-grafting will be an important solution for cell transplantation therapy for PD.

In the present invention, it was shown through animal PD models that, for at least 6 months after transplantation, the co-grafting of ventral midbrain-derived astrocytes with dopamine neural progenitor cells significantly improved the outcomes of cell transplantation therapy for PD models.

Particularly, it shows that the overexpression of Nurr1 and Foxa2 in astrocytes further promotes a neurotrophic action of grafted astrocytes in cell-based therapies. It is expected from this result that the astrocytes co-grafted with the dopamine neural progenitor cells will be very useful in the prevention or treatment of a neurodegenerative disorder.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A cell therapeutic agent comprising:
ventral midbrain-derived astrocytes and dopamine neural progenitor cells.

2. The cell therapeutic agent according to claim 1, wherein, in the astrocytes, Nurr1 (Nuclear receptor related 1) and Foxa2 (forkhead box protein A2) are overexpressed.

3. The cell therapeutic agent according to claim 1, wherein the astrocytes and the neural progenitor cells are mixed at a cell number ratio of 1:1.5˜3.

4. The cell therapeutic agent according to claim 1, which inhibits α-synuclein aggregation and transmission.

5. A method for differentiation into dopamine neurons, comprising:
co-culturing or co-grafting ventral midbrain-derived astrocytes and dopamine neural progenitor cells by mixing.

6. The method according to claim 5, wherein, in the astrocytes, Nurr1 (Nuclear receptor related 1) and Foxa2 (forkhead box protein A2) are overexpressed.

7. The method according to claim 5, wherein the astrocytes and the neural progenitor cells are mixed at a cell number ratio of 1:1.5˜3.

8. A method of treating a neurodegenerative disorder, comprising:
administering a pharmaceutical composition comprising ventral midbrain-derived astrocytes and dopamine neural progenitor cells into a subject.

9. The method according to claim 8, wherein, in the astrocytes, Nurr1 (Nuclear receptor related 1) and Foxa2 (forkhead box protein A2) are overexpressed.

10. The method according to claim 8, wherein the astrocytes and the neural progenitor cells are mixed at a cell number ratio of 1:1.5˜3.

11. The method according to claim 8, wherein the neurodegenerative disorder is selected from Parkinson's disease, dementia, Alzheimer' s disease, Huntington's disease, amyotrophic lateral sclerosis, memory impairment, myasthenia gravis, progressive supranuclear palsy, multiple system atrophy, essential tremor, cortico-basal ganglionic degeneration, diffuse Lewy body disease and Pick's disease.

12. A cell culture obtained by co-culturing ventral midbrain-derived astrocytes and dopamine neural progenitor cells.


SEQUENCE LISTING (124 sequences)

SEQ ID NO: 1 - DNA, 20 bp, Artificial Sequence
  AHR Sense Primer
gtcctcagcaggaacgaaag

SEQ ID NO: 2 - DNA, 20 bp, Artificial Sequence
  AHR Antisense Primer
ccagggaagtccaactgtgt

SEQ ID NO: 3 - DNA, 20 bp, Artificial Sequence
  AQP4 Sense Primer
cggttcatggaaacctcact

SEQ ID NO: 4 - DNA, 20 bp, Artificial Sequence
  AQP4 Antisense Primer
catgctggctccggtataat

SEQ ID NO: 5 - DNA, 20 bp, Artificial Sequence
  ARG1 Sense Primer
tatcggagcgcctttctcta

SEQ ID NO: 6 - DNA, 20 bp, Artificial Sequence
  ARG1 Antisense Primer
acagaccgtgggttcttcac

SEQ ID NO: 7 - DNA, 21 bp, Artificial Sequence
  BDNF Sense Primer
gtgacagtattagcgagtggg

SEQ ID NO: 8 - DNA, 17 bp, Artificial Sequence
  BDNF Antisense Primer
gggtagttcggcattgc

SEQ ID NO: 9 - DNA, 20 bp, Artificial Sequence
  CCL17 Sense Primer
aatgtaggccgagagtgctg

SEQ ID NO: 10 - DNA, 20 bp, Artificial Sequence
  CCL17 Antisense Primer
catgcttgtctttggggtct

SEQ ID NO: 11 - DNA, 20 bp, Artificial Sequence
  CCL22 Sense Primer
ttcttgctgtggcacttcag

SEQ ID NO: 12 - DNA, 20 bp, Artificial Sequence
  CCL22 Antisense Primer
cttcttcacccagagcatcc

SEQ ID NO: 13 - DNA, 20 bp, Artificial Sequence
  COL6A2 Sense Primer
atggacagaagggcaaactg

SEQ ID NO: 14 - DNA, 20 bp, Artificial Sequence
  COL6A2 Antisense Primer
cttgcctcctttcactcctg

SEQ ID NO: 15 - DNA, 20 bp, Artificial Sequence
  CXCL9 Sense Primer
ctcatgggcatcatcttcct

SEQ ID NO: 16 - DNA, 20 bp, Artificial Sequence
  CXCL9 Antisense Primer
tcagcttcttcacccttgct

SEQ ID NO: 17 - DNA, 20 bp, Artificial Sequence
  CXCL10 Sense Primer
tcgtgctgctgagtctgagt

SEQ ID NO: 18 - DNA, 20 bp, Artificial Sequence
  CXCL10 Antisense Primer
ggctcaccgctttcaataag

SEQ ID NO: 19 - DNA, 20 bp, Artificial Sequence
  CXCL11 Sense Primer
tatgatcatctgggccacaa

SEQ ID NO: 20 - DNA, 20 bp, Artificial Sequence
  CXCL11 Antisense Primer
ccaggcacctttgtccttta

SEQ ID NO: 21 - DNA, 20 bp, Artificial Sequence
  EPO Sense Primer
ccagccaccagagagtcttc

SEQ ID NO: 22 - DNA, 20 bp, Artificial Sequence
  EPO Antisense Primer
tgcagaaagtatccgctgtg

SEQ ID NO: 23 - DNA, 20 bp, Artificial Sequence
  FGF8 Sense Primer
tgttgcacttgctggttctc

SEQ ID NO: 24 - DNA, 20 bp, Artificial Sequence
  FGF8 Antisense Primer
actcggactctgcttccaaa

SEQ ID NO: 25 - DNA, 20 bp, Artificial Sequence
  FIZZ1 Sense Primer
atctgcgtcttccttctcca

SEQ ID NO: 26 - DNA, 20 bp, Artificial Sequence
  FIZZ1 Antisense Primer
cagtagcagtcatcccagca

SEQ ID NO: 27 - DNA, 20 bp, Artificial Sequence
  FN1 Sense Primer
gaaaggcaaccagcagagtc

SEQ ID NO: 28 - DNA, 20 bp, Artificial Sequence
  FN1 Antisense Primer
ctggagtcaagccagacaca

SEQ ID NO: 29 - DNA, 20 bp, Artificial Sequence
  Foxa2 Sense Primer
gctccctacgccaatatcaa

SEQ ID NO: 30 - DNA, 20 bp, Artificial Sequence
  Foxa2 Antisense Primer
ccggtagaaagggaagaggt

SEQ ID NO: 31 - DNA, 20 bp, Artificial Sequence
  Foxg1 Sense Primer
gaacggcaagtacgagaagc

SEQ ID NO: 32 - DNA, 20 bp, Artificial Sequence
  Foxg1 Antisense Primer
tcacgaagcacttgttgagg

SEQ ID NO: 33 - DNA, 20 bp, Artificial Sequence
  GDNF Sense Primer
cgcccgccgaagaccactcc

SEQ ID NO: 34 - DNA, 20 bp, Artificial Sequence
  GDNF Antisense Primer
gtcgaaggcgaccggcctgc

SEQ ID NO: 35 - DNA, 21 bp, Artificial Sequence
  GFAP Sense Primer
gcagacctcacagacgttgct

SEQ ID NO: 36 - DNA, 21 bp, Artificial Sequence
  GFAP Antisense Primer
aggctggtttctcggatctgg

SEQ ID NO: 37 - DNA, 20 bp, Artificial Sequence
  GLT1 Sense Primer
atcctgggagcagtatgtgg

SEQ ID NO: 38 - DNA, 20 bp, Artificial Sequence
  GLT1 Antisense Primer
ctgacagccctgtgatgaga

SEQ ID NO: 39 - DNA, 20 bp, Artificial Sequence
  GPC4 Sense Primer
agtgccttcagtgctcgatt

SEQ ID NO: 40 - DNA, 20 bp, Artificial Sequence
  GPC4 Antisense Primer
cttcccgttccaacagtcat

SEQ ID NO: 41 - DNA, 20 bp, Artificial Sequence
  GPC6 Sense Primer
agccagatacctgcctgaga

SEQ ID NO: 42 - DNA, 20 bp, Artificial Sequence
  GPC6 Antisense Primer
tcattgccattgtacgcatt

SEQ ID NO: 43 - DNA, 20 bp, Artificial Sequence
  GPX3 Sense Primer
accaatttggcaaacaggag

SEQ ID NO: 44 - DNA, 20 bp, Artificial Sequence
  GPX3 Antisense Primer
agcggatgtcatggatcttc

SEQ ID NO: 45 - DNA, 20 bp, Artificial Sequence
  Hevin Sense Primer
attggcaaccagaaggacac

SEQ ID NO: 46 - DNA, 20 bp, Artificial Sequence
  Hevin Antisense Primer
ggttctcactctcgccagtc

SEQ ID NO: 47 - DNA, 20 bp, Artificial Sequence
  IFN-alpha Sense Primer
ggtggtggtgagctactggt

SEQ ID NO: 48 - DNA, 20 bp, Artificial Sequence
  IFN-alpha Antisense Primer
ttgagccttctggatctgct

SEQ ID NO: 49 - DNA, 20 bp, Artificial Sequence
  IFN-beta Sense Primer
ctgccctctccatcgactac

SEQ ID NO: 50 - DNA, 20 bp, Artificial Sequence
  IFN-beta Antisense Primer
acccagtgctggagaaattg

SEQ ID NO: 51 - DNA, 20 bp, Artificial Sequence
  IL-10 Sense Primer
cctgctcttactggctggag

SEQ ID NO: 52 - DNA, 20 bp, Artificial Sequence
  IL-10 Antisense Primer
tgtccagctggtccttcttt

SEQ ID NO: 53 - DNA, 20 bp, Artificial Sequence
  IL-12b Sense Primer
accctcacctgtgacagtcc

SEQ ID NO: 54 - DNA, 20 bp, Artificial Sequence
  IL-12b Antisense Primer
ttcttgtggagcagcagatg

SEQ ID NO: 55 - DNA, 20 bp, Artificial Sequence
  IL-13 Sense Primer
atcgaggagctgagcaacat

SEQ ID NO: 56 - DNA, 20 bp, Artificial Sequence
  IL-13 Antisense Primer
cgaggccttttggttacaga

SEQ ID NO: 57 - DNA, 20 bp, Artificial Sequence
  IL-19 Sense Primer
agaaggcatgaaggcacagt

SEQ ID NO: 58 - DNA, 20 bp, Artificial Sequence
  IL-19 Antisense Primer
tcacgcagcacacatctaca

SEQ ID NO: 59 - DNA, 20 bp, Artificial Sequence
  IL-1R2 Sense Primer
catgggagatgcaggctatt

SEQ ID NO: 60 - DNA, 20 bp, Artificial Sequence
  IL-1R2 Antisense Primer
acaccttgcacgggactatc

SEQ ID NO: 61 - DNA, 20 bp, Artificial Sequence
  IL-1RN Sense Primer
gaaaagaccctgcaagatgc

SEQ ID NO: 62 - DNA, 20 bp, Artificial Sequence
  IL-1RN Antisense Primer
gatgcccaagaacacattcc

SEQ ID NO: 63 - DNA, 20 bp, Artificial Sequence
  IL-1beta Sense Primer
ctgtgactcgtgggatgatg

SEQ ID NO: 64 - DNA, 20 bp, Artificial Sequence
  IL-1beta Antisense Primer
gggattttgtcgttgcttgt

SEQ ID NO: 65 - DNA, 20 bp, Artificial Sequence
  IL-2RA Sense Primer
ccagagagtgaggcttcctg

SEQ ID NO: 66 - DNA, 20 bp, Artificial Sequence
  IL-2RA Antisense Primer
actcaggaggaggatgctga

SEQ ID NO: 67 - DNA, 20 bp, Artificial Sequence
  IL-4 Sense Primer
tccttacggcaacaaggaac

SEQ ID NO: 68 - DNA, 20 bp, Artificial Sequence
  IL-4 Antisense Primer
gtgagttcagaccgctgaca

SEQ ID NO: 69 - DNA, 21 bp, Artificial Sequence
  IL-6 Sense Primer
atggatgctaccaaactggat

SEQ ID NO: 70 - DNA, 21 bp, Artificial Sequence
  IL-6 Antisense Primer
tgaaggactctggctttgtct

SEQ ID NO: 71 - DNA, 20 bp, Artificial Sequence
  iNOS Sense Primer
caccttggagttcacccagt

SEQ ID NO: 72 - DNA, 20 bp, Artificial Sequence
  iNOS Antisense Primer
accactcgtacttgggatgc

SEQ ID NO: 73 - DNA, 20 bp, Artificial Sequence
  ITGalphaM Sense Primer
ttaccggactgtgtggacaa

SEQ ID NO: 74 - DNA, 20 bp, Artificial Sequence
  ITGalphaM Antisense Primer
agtctcccaccaccaaagtg

SEQ ID NO: 75 - DNA, 20 bp, Artificial Sequence
  ITGbeta4 Sense Primer
gaaggaactgcaggtgaagc

SEQ ID NO: 76 - DNA, 20 bp, Artificial Sequence
  ITGbeta4 Antisense Primer
gcgatgcggatatctcattt

SEQ ID NO: 77 - DNA, 20 bp, Artificial Sequence
  Lmx1a Sense Primer
ccccaaaatccggaattact

SEQ ID NO: 78 - DNA, 20 bp, Artificial Sequence
  Lmx1a Antisense Primer
ctcagggtcagcaaaaggag

SEQ ID NO: 79 - DNA, 20 bp, Artificial Sequence
  NF1A Sense Primer
acccgagtaccgagaggatt

SEQ ID NO: 80 - DNA, 20 bp, Artificial Sequence
  NF1A Antisense Primer
actgtggggacttcacaagg

SEQ ID NO: 81 - DNA, 21 bp, Artificial Sequence
  NT3 Sense Primer
ggtcagaattccagccgatga

SEQ ID NO: 82 - DNA, 21 bp, Artificial Sequence
  NT3 Antisense Primer
ggcacacacacaggaagtgtc

SEQ ID NO: 83 - DNA, 20 bp, Artificial Sequence
  Nurr1 Sense Primer
accctcttctggacacatgg

SEQ ID NO: 84 - DNA, 20 bp, Artificial Sequence
  Nurr1 Antisense Primer
gccatcaccacaagacacac

SEQ ID NO: 85 - DNA, 20 bp, Artificial Sequence
  PAX6 Sense Primer
aacaacctgcctatgcaacc

SEQ ID NO: 86 - DNA, 20 bp, Artificial Sequence
  PAX6 Antisense Primer
acttggacgggaactgacac

SEQ ID NO: 87 - DNA, 20 bp, Artificial Sequence
  Pitx3 Sense Primer
ccaccctacgaggaggtgta

SEQ ID NO: 88 - DNA, 19 bp, Artificial Sequence
  Pitx3 Antisense Primer
gggcggtgagaatacaggt

SEQ ID NO: 89 - DNA, 20 bp, Artificial Sequence
  PRNP Sense Primer
gtggctacatgttggggagt

SEQ ID NO: 90 - DNA, 20 bp, Artificial Sequence
  PRNP Antisense Primer
gtgaagttctcccccttggt

SEQ ID NO: 91 - DNA, 21 bp, artificial sequence
  S100beta Sense Primer
ggcggcaaaaggtgaccagga

SEQ ID NO: 92 - DNA, 20 bp, Artificial Sequence
  S100beta Antisense Primer
gccctcatgtctgccacggg

SEQ ID NO: 93 - DNA, 20 bp, Artificial Sequence
  SERPIN B1B Sense Primer
acccgagtaccgagaggatt

SEQ ID NO: 94 - DNA, 20 bp, Artificial Sequence
  SERPIN B1B Antisense Primer
actgtggggacttcacaagg

SEQ ID NO: 95 - DNA, 20 bp, Artificial Sequence
  SHH Sense Primer
aaaagctgacccctttagcc

SEQ ID NO: 96 - DNA, 20 bp, Artificial Sequence
  SHH Antisense Primer
tgcacctctgagtcatcagc

SEQ ID NO: 97 - DNA, 20 bp, Artificial Sequence
  SOD3 Sense Primer
gacctggagatctggatgga

SEQ ID NO: 98 - DNA, 20 bp, Artificial Sequence
  SOD3 Antisense Primer
gtggttggaggtgttctgct

SEQ ID NO: 99 - DNA, 20 bp, Artificial Sequence
  SPO1 Sense Primer
taccatgtcggagtggatca

SEQ ID NO: 100 - DNA, 20 bp, Artificial Sequence
  SPO1 Antisense Primer
actcaccccactcagtcacc

SEQ ID NO: 101 - DNA, 20 bp, Artificial Sequence
  SPO2 Sense Primer
agtggagccagacagcattt

SEQ ID NO: 102 - DNA, 20 bp, Artificial Sequence
  SPO2 Antisense Primer
ctcctgctgcttcgatctct

SEQ ID NO: 103 - DNA, 20 bp, Artificial Sequence
  TGF-B Sense Primer
atacgcctgagtggctgtct

SEQ ID NO: 104 - DNA, 20 bp, Artificial Sequence
  TGF-B Antisense Primer
tgggactgatcccattgatt

SEQ ID NO: 105 - DNA, 20 bp, Artificial Sequence
  Thbs1 Sense Primer
ccagttcaaccaacgtcctt

SEQ ID NO: 106 - DNA, 20 bp, Artificial Sequence
  Thbs1 Antisense Primer
ttgcgaatgctgtcctgtag

SEQ ID NO: 107 - DNA, 20 bp, Artificial Sequence
  TNF-alpha Sense Primer
agatgtggaactggcagagg

SEQ ID NO: 108 - DNA, 20 bp, Artificial Sequence
  TNF-alpha Antisense Primer
cccatttgggaacttctcct

SEQ ID NO: 109 - DNA, 20 bp, artificial sequence
  TXNIP Sense Primer
ggcacacttaccttgccaat

SEQ ID NO: 110 - DNA, 20 bp, Artificial Sequence
  TXNIP Antisense Primer
actaggggcagatcgaggat

SEQ ID NO: 111 - DNA, 20 bp, Artificial Sequence
  Vimentin Sense Primer
agatcgatgtggacgtttcc

SEQ ID NO: 112 - DNA, 21 bp, Artificial Sequence
  Vimentin Antisense Primer
cacactgtctccggtattcgt

SEQ ID NO: 113 - DNA, 20 bp, Artificial Sequence
  Wnt1 Sense Primer
cttcggcaagatcgtcaacc

SEQ ID NO: 114 - DNA, 21 bp, Artificial Sequence
  Wnt1 Antisense Primer
gcgaagatgaacgctgtttct

SEQ ID NO: 115 - DNA, 22 bp, Artificial Sequence
  Wnt3a Sense Primer
gaacgcgacctggtctactacg

SEQ ID NO: 116 - DNA, 22 bp, Artificial Sequence
  Wnt3a Antisense Primer
gttaggttcgcagaagttgggt

SEQ ID NO: 117 - DNA, 20 bp, Artificial Sequence
  Wnt4 Sense Primer
gccacgcactaaaggagaag

SEQ ID NO: 118 - DNA, 20 bp, Artificial Sequence
  Wnt4 Antisense Primer
ggccttagacgtcttgttcg

SEQ ID NO: 119 - DNA, 21 bp, Artificial Sequence
  Wnt5a Sense Primer
aataaccctgttcagatgtca

SEQ ID NO: 120 - DNA, 20 bp, Artificial Sequence
  Wnt5a Antisense Primer
tactgcatgtggtcctgata

SEQ ID NO: 121 - DNA, 20 bp, Artificial Sequence
  Wnt7a Sense Primer
cgagagctaggctacgtgct

SEQ ID NO: 122 - DNA, 20 bp, artificial sequence
  Wnt7a Antisense Primer
ctgaggggctgtcttattgc

SEQ ID NO: 123 - DNA, 20 bp, Artificial Sequence
  YM1 Sense Primer
tcgtgagaagctcattgtgg

SEQ ID NO: 124 - DNA, 20 bp, Artificial Sequence
  YM1 Antisense Primer
aacccatacattgccctgaa